SEQUENCE LISTING (152 sequences)

SEQ ID NO: 1 - protein, 98 aa, Avian orthoreovirus
  source 1..98: note = ARV_p10_Avian orthoreovirus
MLRMPPGSCNGATAVFGNVHCQAAQNTAGGDLQATSSIIAYWPYLAAGGGFLLIVIIFALLYCCKAKVKADAARSVFHRELVALSSGKHNAMAPPYDV

SEQ ID NO: 2 - protein, 95 aa, Nelson Bay orthoreovirus
  source 1..95: note = NB Nelson Bay orthoreovirus
MSSDCAKIVSVFGSVHCQSSKNSAGGDLQATSVFTTYWPHFAIGCGIIVVILLLGLFYCCYLKWKTSQVKHTYRRELIALTRSHVHSTPSGISYV

SEQ ID NO: 3 - protein, 113 aa, Broome reovirus
  source 1..113: note = BroV_p13_Broome orthoreovirus
MGSGPSNFVNKVDGASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 4 - protein, 125 aa, Reptilian orthoreovirus
  source 1..125: note = RRV_p14_Reptilian orthoreovirus
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 5 - protein, 141 aa, Baboon orthoreovirus
  source 1..141: note = BRV_p15_Baboon orthoreovirus
MGQRHSIVQPPAPPPNAFVEIVSSSTGIIIAVGIFAFIFSFLYKLLQWYNRKSKNKKRKEQIREQIELGLLSYGAGVASLPLLNVIAHNPGSVISATPIYKGPCTGVPNSRLLQITSGTAEENTRIILNHDGRNPDGSINV

SEQ ID NO: 6 - protein, 146 aa, Aquareovirus-C
  source 1..146: note = AqRV-C_p16 Aquareovirus-C
MPCQDTVSLSIQHTSVYVQHSCCVSTTTSASTSATALGLGCLACGIVGVLVVAGGLCCLINGRCPSCRRLALRSRSWKPPPTSLCTNQPLAFNLRDLTRSNIRCTSDPRSVELLSDVHSVVSHRECPPAYDSLDFEPTEYTPEAFQ

SEQ ID NO: 7 - protein, 198 aa, Aquareovirus-A
  source 1..198: note = AqRV-A_p22 Aquareovirus-A
MGNTISNTVQYTVLQIDRSCCIKTSLTATSEATSWAIPPLAICCCCCICCTGGLYLVHSGRFPGLSRRLDVLGGSGSTPKHSLRSHRHPKPRVHRVSFSDSSDSSDISDLELPRHGSHPLAHSFRPEVDRHRPRPSTQVQQTSFIPLVPLRSGSSLDDGIVRSQPSRDSRPHEQFEDWLQQAHLLRPGRVSGSTNPFT

SEQ ID NO: 8 - protein, 188 aa, Atlantic halibut reovirus
  source 1..188: note = Halibut
MPCGDTVSTTVQYTVLQIDRSCCISTSLTATSEATSWALPPLCICCCCLVCTGLGIYAIQRGHCPGISRRLDVLRSSGSSSKQPVCDQRRERSRIYRPRSSDSFGFDDQSTLDLHRDSPRVLENQFRLENNRSRSSPKPLSVQPSAFDESIRLHIQSLIRTQPQHDDSNRNQDSLIHEHIPRRGNPFI

SEQ ID NO: 9 - protein, 125 aa, Mahlapitisi orthoreovirus
  source 1..125: note = MaRV_Mahlapitisi orthoreovirus
MGSGPSNFVNHASGEAIISGLSDQTNRLGSLLSQNVYNIIYFFVIGGLILSAGYGLYKYCKYKQRRAKNLTRQLSRELIDLNRKIEHISGGKIPATKPSAPRYTPPCYKEPIYNEVCEGGFYGNC

SEQ ID NO: 10 - protein, 97 aa, Muscovy duck orthoreovirus
  source 1..97: note = MdRV_Muscovy duck orthoreovirus
MADGACNHATSIFGAVYCQISQNIAHGNIDSYTSWTSYLPPILGGGFGLIVLLVLVVLIVYCCKHSKILSAVKATDSAVTTLLRDVAPANPDPVQVV

SEQ ID NO: 11 - protein, 135 aa, Reptilian Testudine orthoreovirus
  source 1..135: note = RRVtes_Reptilian Testudine orthoreovirus
MGNGPSNFVNHSPAEAILTGLDKNTHSITSTFTNGIKELLVGLLVLILFFVAAGAACWYWRKRRAKRRRLTKYQIRFLNDFSRSARLNQTPIPVASKRETWPQKTRSVSPPPYTANYVNVNDYDEAESAPTFRRY

SEQ ID NO: 12 - protein, 98 aa, Piscine Orthoreovirus
  source 1..98: note = PsRV_Piscine Orthoreovirus
MPRLPGGSCNGATSVFGSVHCQAAQNTAGGDLQATSSLVSYWPYLAAGGSLLLIVVLVALFFCCRAKIKADATKNVFRRELIALTTKSGHDRPPSYEV

SEQ ID NO: 13 - protein, 98 aa, Avian orthoreovirus turkey
  source 1..98: note = ARVtu - Avian orthoreovirus turkey
MSRPSSGSCNGALAVFGNVHCQAAQNSAGGDLQATSSLIAYWPYLAAGGGVLLLLIVVVAVIYCKAKVKADAARNVFFRELVALNEVKCNAVPPSYRV

SEQ ID NO: 14 - protein, 80 aa, Feline Rotavirus Species I
  source 1..80: note = Feline Rotavirus Species I NSP-1 (FeRVI)
MGGRISQQLSQQNTYHIASGNSQIYSQDQKTNQQVAVAFEVCHLSILFIIALALVVHVTCSARKRGCAVKSFEKPFLTNV

SEQ ID NO: 15 - protein, 79 aa, Canine Rotavirus Species I
  source 1..79: note = Canine Rotavirus Species I NSP-1 (CaRVI)
MGNTYNVHNNQQVSSNTVHGSGQIHSEDQKTSSQITTIVQFSNLSLLFLIALFLFISLLFKCDKNRKKQKWNTREIIEL

SEQ ID NO: 16 - protein, 84 aa, Gallinaceous Rotavirus Species G
  source 1..84: note = Gallinaceous Rotavirus Species G NSP-1 (GaRVG)
KLNLQDQKTTSLESTQLLLGIGAIVVVALIILLIFSLILNCYLCSKLKRKNGYLKRERKISNCRDKGLDKLILSKSDDIASSCV

SEQ ID NO: 17 - protein, 104 aa, Avian Rotavirus Species G
  source 1..104: note = Avian Rotavirus Species G NSP-1 (AvRVG)
MGSHQSSYQVNNQNTIISNSKNLDFKPSTSSSLSQVNLFFVIGAAVGVFLLTVLIISIILNIYLCRRLKNGRKHDGRTSFQSGTTSRHNAKMDEREHLQSNTNV

SEQ ID NO: 18 - protein, 101 aa, Caprine Rotavirus Species B
  source 1..101: note = Caprine Rotavirus Species B NSP-1 (CaRVB)
MGSSQSSLQSQVHSTNIHSQHSSIHLQGTSTANFTTQHIILTVGAALIALLLTSLIFSCICNCYLYSKLRNGFQTVSQHVRRKERSHTNIPGQQIRPDMYV

SEQ ID NO: 19 - protein, 101 aa, Porcine Rotavirus Species B
  source 1..101: note = Porcine Rotavirus Species B NSP-1 (PoRVB)
MGNRQSSLQSQTHRTDINSHNSNIYLQSASSAEFRTQHILVVAGAALIALLFAFLVSSLVCNCYLLRRLRNGPRKIYRTGKIQEGSYSSLSKQFIRPDHFV

SEQ ID NO: 20 - protein, 107 aa, Human Rotavirus Species B
  source 1..107: note = Human Rotavirus Species B NSP-1 (HuRVB)
MGNRQSSAQLNSHLTHINSQNSNLFISDSKTAVFHTQHILLAAGVGIIATLLVLLLCSCVLNCYLCRKLKRTNGVSSLLERNIRQNGSSAKIYVKPVMQSSTIIEEA

SEQ ID NO: 21 - protein, 116 aa, synthetic construct
  source 1..116: note = P15-PsRV-RRV
MGQRHSIVQPPAPPPNAFVESGYWPYLAAGGSLLLIVVLVALFFCCGSKHLQKRRERARQLSEFQKRYLRNSYRLSEVQRPISLHEYEDPYEPPSRKKAPPPPYSTYVNIDDVSAV

SEQ ID NO: 22 - protein, 95 aa, synthetic construct
  source 1..95: note = P13-PsRV-MdRV
MGSGPSNFVNKVDGASAPIKEHAIPSLTSDLKDSGYWPYLAAGGSLLLIVVLVALFFCCGSKHSKILSAVKATDSAVTTLLRDVAPANPDPVQVV

SEQ ID NO: 23 - protein, 100 aa, synthetic construct
  source 1..100: note = P10_MdRV_NB
MLRMPPGSCNGATAVFGNVHCQAAQNTAGGDLQATSSIIASGVLGGGFGLIVLLVPVVLIVYCCGSLKWKTSQVKHTYRRELIALTRSHVHSTPSGISYV

SEQ ID NO: 24 - protein, 127 aa, synthetic construct
  source 1..127: note = P13_MdRV_MARV
MGSGPSNFVNKVDGASAPIKEHAIPSLTSDLKDSGILGGGFGLIVLLVLVVLIVYCCGSKYCKYKQRRAKNLTRQLSRELIDLNRKIEHISGGKIPATKPSAPRYTPPCYKEPIYNEVCEGGFYGNC

SEQ ID NO: 25 - protein, 100 aa, synthetic construct
  source 1..100: note = P10_MdRV_NB
MLRMPPGSCNGATAVFGNVHCQAAQNTAGGDLQATSSIIASGILGGGFGLIVLLVLVVLIVYCCGSLKWKTSQVKHTYRRELIALTRSHVHSTPSGISYV

SEQ ID NO: 26 - protein, 96 aa, synthetic construct
  source 1..96: note = P22_P10_MdRV
MGNTISNTVQYTVLQIDRSCCIKTSLTATSEATSSGYWPYLAAGGGFLLIVIIFALLYCCGSKHSKILSAVKATDSAVTTLLRDVAPANPDPVQVV

SEQ ID NO: 27 - protein, 102 aa, synthetic construct
  source 1..102: note = P10_PsRV_ARVch
MLRMPPGSCNGATAVFGNVHCQAAQNTAGGDLQATSSIIASGYWPYLAAGGSLLLIVVLVALFFCCGSKAKVKADAARSVFHRELVALSSGKHNAMAPPYDV

SEQ ID NO: 28 - protein, 96 aa, synthetic construct
  source 1..96: note = Halibut_RRVtes_ARVch
MPCGDTVSTTVQYTVLQIDRSCCISTSLTATSEATSSGLLVGLLVLILFFVAAGAACWYWGSKAKVKADAARSVFHRELVALSSGKHNAMAPPYDV

SEQ ID NO: 29 - protein, 98 aa, synthetic construct
  source 1..98: note = MARV_pNB_p10
MGSGPSNFVNHASGEAIISGLSDQTNRLGSLLSQNSGYWPHFAIGCGIIVVILLLGLFYCCYGSKAKVKADAARSVFHRELVALSSGKHNAMAPPYDV

SEQ ID NO: 30 - protein, 93 aa, synthetic construct
  source 1..93: note = p13_p13_p10
MGSGPSNFVNKVDGASAPIKEHAIPSLTSDLKDSGYLYTIVTAVILLVILWFLYRYYGSKAKVKADAARSVFHRELVALSSGKHNAMAPPYDV

SEQ ID NO: 31 - protein, 93 aa, synthetic construct
  source 1..93: note = p13_p13_PsRV
MGSGPSNFVNKVDGASAPIKEHAIPSLTSDLKDSGYLYTIVTAVILLVILWFLYRYYGSRAKIKADATKNVFRRELIALTTKSGHDRPPSYEV

SEQ ID NO: 32 - protein, 96 aa, synthetic construct
  source 1..96: note = p13_pNB_p10
MGSGPSNFVNKVDGASAPIKEHAIPSLTSDLKDSGYWPHFAIGCGIIVVILLLGLFYCCYGSKAKVKADAARSVFHRELVALSSGKHNAMAPPYDV

SEQ ID NO: 33 - protein, 96 aa, synthetic construct
  source 1..96: note = p13_pNB_PsRV
MGSGPSNFVNKVDGASAPIKEHAIPSLTSDLKDSGYWPHFAIGCGIIVVILLLGLFYCCYGSRAKIKADATKNVFRRELIALTTKSGHDRPPSYEV

SEQ ID NO: 34 - protein, 93 aa, synthetic construct
  source 1..93: note = p13_pVC_pNB
MGSGPSNFVNKVDGASAPIKEHAIPSLTSDLKDSGIWEVIAGLVALLIFLAIGFWLFGSLKWKTSQVKHTYRRELIALTRSHVHSTPSGISYV

SEQ ID NO: 35 - protein, 93 aa, synthetic construct
  source 1..93: note = p13_RRVlep_MdRV
MGSGPSNFVNKVDGASAPIKEHAIPSLTSDLKDSGIWEVIAGLVALLTFLAFGFWLFGSKHSKILSAVKATDSAVTTLLRDVAPANPDPVQVV

SEQ ID NO: 36 - protein, 93 aa, synthetic construct
  source 1..93: note = p13_RRVlep_pNB
MGSGPSNFVNKVDGASAPIKEHAIPSLTSDLKDSGIWEVIAGLVALLTFLAFGFWLFGSLKWKTSQVKHTYRRELIALTRSHVHSTPSGISYV

SEQ ID NO: 37 - protein, 93 aa, synthetic construct
  source 1..93: note = p13_RRVtes_ARVch
MGSGPSNFVNKVDGASAPIKEHAIPSLTSDLKDSGLLVGLLVLILFFVAAGAACWYWGSKAKVKADAARSVFHRELVALSSGKHNAMAPPYDV

SEQ ID NO: 38 - protein, 93 aa, synthetic construct
  source 1..93: note = p13_RRVtes_ARVtu
MGSGPSNFVNKVDGASAPIKEHAIPSLTSDLKDSGLLVGLLVLILFFVAAGAACWYWGSKAKVKADAARNVFFRELVALNEVKCNAVPPSYRV

SEQ ID NO: 39 - protein, 80 aa, synthetic construct
  source 1..80: note = p15_MdRV_MdRV
MGQRHSIVQPPAPPPNAFVESGILGGGFGLIVLLVLVVLIVYCCGSKHSKILSAVKATDSAVTTLLRDVAPANPDPVQVV

SEQ ID NO: 40 - protein, 84 aa, synthetic construct
  source 1..84: note = p15_p15_PsRV
MGQRHSIVQPPAPPPNAFVESGIVSSSTGIIIAVGIFAFIFSFLYKLLGSRAKIKADATKNVFRRELIALTTKSGHDRPPSYEV

SEQ ID NO: 41 - protein, 83 aa, synthetic construct
  source 1..83: note = p15_pNB_PsRV
MGQRHSIVQPPAPPPNAFVESGYWPHFAIGCGIIVVILLLGLFYCCYGSRAKIKADATKNVFRRELIALTTKSGHDRPPSYEV

SEQ ID NO: 42 - protein, 80 aa, synthetic construct
  source 1..80: note = p15_RRVtes_ARVtu
MGQRHSIVQPPAPPPNAFVESGLLVGLLVLILFFVAAGAACWYWGSKAKVKADAARNVFFRELVALNEVKCNAVPPSYRV

SEQ ID NO: 43 - protein, 80 aa, synthetic construct
  source 1..80: note = p15_RRVtes_ARVch
MGQRHSIVQPPAPPPNAFVESGLLVGLLVLILFFVAAGAACWYWGSKAKVKADAARSVFHRELVALSSGKHNAMAPPYDV

SEQ ID NO: 44 - protein, 97 aa, synthetic construct
  source 1..97: note = p16_ARVch_ARVch
MPCQDTVSLSIQHTSVYVQHSCCVSTTTSASTSATSGYWPYLAAGGGFLLIVIIFALLYCCGSKAKVKADAARSVFHRELVALSSGKHNAMAPPYDV

SEQ ID NO: 45 - protein, 95 aa, synthetic construct
  source 1..95: note = p16_RRVlep_p10
MPCQDTVSLSIQHTSVYVQHSCCVSTTTSASTSATSGIWEVIAGLVALLTFLAFGFWLFGSKAKVKADAARSVFHRELVALSSGKHNAMAPPYDV

SEQ ID NO: 46 - protein, 94 aa, synthetic construct
  source 1..94: note = p22_MaRV_ARVtu
MGNTISNTVQYTVLQIDRSCCIKTSLTATSEATSSGVYNIIYFFVIGGLILSAGYGLYGSKAKVKADAARNVFFRELVALNEVKCNAVPPSYRV

SEQ ID NO: 47 - protein, 98 aa, synthetic construct
  source 1..98: note = p22_p15_MdRV
MGNTISNTVQYTVLQIDRSCCIKTSLTATSEATSSGIVSSSTGIIIAVGIFAFIFSFLYKLLGSKHSKILSAVKATDSAVTTLLRDVAPANPDPVQVV

SEQ ID NO: 48 - protein, 98 aa, synthetic construct
  source 1..98: note = pNB_ARVch_PsRV
MSSDCAKIVSVFGSVHCQSSKNSAGGDLQATSVFTTSGYWPYLAAGGGFLLIVIIFALLYCCGSRAKIKADATKNVFRRELIALTTKSGHDRPPSYEV

SEQ ID NO: 49 - protein, 98 aa, synthetic construct
  source 1..98: note = pNB_p10_p10
MSSDCAKIVSVFGSVHCQSSKNSAGGDLQATSVFTTSGYWPYLAAGGGFLLIVIIFALLYCCGSKAKVKADAARSVFHRELVALSSGKHNAMAPPYDV

SEQ ID NO: 50 - protein, 96 aa, synthetic construct
  source 1..96: note = pNB_p13_ARVch
MSSDCAKIVSVFGSVHCQSSKNSAGGDLQATSVFTTSGYLYTIVTAVILLVILWFLYRYYGSKAKVKADAARSVFHRELVALSSGKHNAMAPPYDV

SEQ ID NO: 51 - protein, 97 aa, synthetic construct
  source 1..97: note = pNB_p22_PsRV
MSSDCAKIVSVFGSVHCQSSKNSAGGDLQATSVFTTSGWAIPPLAICCCCCICCTGGLYLVGSRAKIKADATKNVFRRELIALTTKSGHDRPPSYEV

SEQ ID NO: 52 - protein, 97 aa, synthetic construct
  source 1..97: note = pVC_PsRV_MdRV
MGSGPSNFVNHAPGEAIVTGLEKGADKVAGTISHTSGYWPYLAAGGSLLLIVVLVALFFCCGSKHSKILSAVKATDSAVTTLLRDVAPANPDPVQVV

SEQ ID NO: 53 - protein, 95 aa, synthetic construct
  source 1..95: note = pVC_RRVtes_pNB
MGSGPSNFVNHAPGEAIVTGLEKGADKVAGTISHTSGLLVGLLVLILFFVAAGAACWYWGSLKWKTSQVKHTYRRELIALTRSHVHSTPSGISYV

SEQ ID NO: 54 - protein, 21 aa, Homo sapiens
  source 1..21: note = VCAM-1
GSRKANMKGSYSLVEAQKSKV

SEQ ID NO: 55 - protein, 71 aa, Homo sapiens
  source 1..71: note = P-Selectin
GSRLSRKGHMYPVRNYSPTEMVCISSLLPDGGEGPSATANGGLSKAKSPGLTPEPREDREGDDLTLHSFLP

SEQ ID NO: 56 - protein, 19 aa, Homo sapiens
  source 1..19: note = L-Selectin
GSRRLKKGKKSKRSMNDPY

SEQ ID NO: 57 - protein, 85 aa, Homo sapiens
  source 1..85: note = CRTAM
GSKLRKAHVIWKKENEVSEHTLESYRSRSNNEETSSEEKNGQSSHPMRCMNYITKLYSEAKTKRKENVQHSKLEEKHIQVPESIV

SEQ ID NO: 58 - protein, 71 aa, Homo sapiens
  source 1..71: note = ITGB2
GSIAAIVGGTVAGIVLIGILLLVIWKALIHLSDLREYRRFEKEKLKSQWNNDNPLFKSATTTVMNPKFAES

SEQ ID NO: 59 - protein, 72 aa, Homo sapiens
  source 1..72: note = ITGB1
GSIIPIVAGVVAGIVLIGLALLLIWKLLMIIHDRREFAKFEKEKMNAKWDTGENPIYKSAVTTVVNPKYEGK

SEQ ID NO: 60 - protein, 34 aa, Homo sapiens
  source 1..34: note = E-Selectin
GSRKCLRKAKKFVPASSCQSLESDGSYQKPSYIL

SEQ ID NO: 61 - protein, 153 aa, Homo sapiens
  source 1..153: note = P-Cadherin
GSKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 62 - protein, 34 aa, Homo sapiens
  source 1..34: note = CD103
GSKCGFFKRKYQQLNLESIRKAQLKSENLLEEEN

SEQ ID NO: 63 - protein, 175 aa, Homo sapiens
  source 1..175: note = E-Cadherin
GSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 64 - protein, 162 aa, Homo sapiens
  source 1..162: note = N-Cadherin
GSKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 65 - protein, 47 aa, Homo sapiens
  source 1..47: note = MUC4
GSAFFGIFFGALGGLLLLGVGTFVVLRFWGCSGARFSYFLNSAEALP

SEQ ID NO: 66 - protein, 52 aa, Homo sapiens
  source 1..52: note = ICAM1
IVIITVVAAAVIMGTAGLSTYLYNRQRKIKKYRLQQAQKGTPMKPNTQATPP

SEQ ID NO: 67 - protein, 113 aa, synthetic construct
  source 1..113: note = Mut 1
MAAAASNFVNKVDGASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 68 - DNA, 348 bp, synthetic construct
  source 1..348: mol_type = other DNA; note = Mut 1
gccaccatggctgccgcagcgagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 69 - DNA, 348 bp, synthetic construct
  source 1..348: mol_type = other DNA; note = Mut 2
gccaccatgggttctggacctgctgccgcagcgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 70 - protein, 113 aa, synthetic construct
  source 1..113: note = Mut 3
MGSGPSNFVAAAAGASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 71 - DNA, 348 bp, synthetic construct
  source 1..348: mol_type = other DNA; note = Mut 3
gccaccatgggttctggacctagcaattttgtggctgccgcagcgggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 72 - protein, 113 aa, synthetic construct
  source 1..113: note = Mut 4
MGSGPSNFVNKVDAAAAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 73 - DNA, 348 bp, synthetic construct
  source 1..348: mol_type = other DNA; note = Mut 4
gccaccatgggttctggacctagcaattttgtgaacaaagtagatgctgccgcagcgcccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 74 - protein, 113 aa, synthetic construct
  source 1..113: note = Mut 5
MGSGPSNFVNKVDGASAAAAAHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 75 - DNA, 348 bp, synthetic construct
  source 1..348: mol_type = other DNA; note = Mut 5
gccaccatgggttctggacctagcaattttgtgaacaaagtagatggggccagcgccgctgccgcagcgcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 76 - protein, 113 aa, synthetic construct
  source 1..113: note = Mut 6
MGSGPSNFVNKVDGASAPIKEAAAASLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 77 - DNA, 348 bp, synthetic construct
  source 1..348: mol_type = other DNA; note = Mut 6
gccaccatgggttctggacctagcaattttgtgaacaaagtagatggggccagcgcccccattaaggaggctgccgcagcgtcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 78 - protein, 113 aa, synthetic construct
  source 1..113: note = Mut 7
MGSGPSNFVNKVDGASAPIKEHAIPAAAADLKDYLYTIVTAVILLVILWFLYRYYKDKKARKKKEDILLRLYGRGLNLSRLDPSVICSLGGSAPNLQHRGLERTEDKLVNPFI

SEQ ID NO: 79 - DNA, 348 bp, synthetic construct
  source 1..348: mol_type = other DNA; note = Mut 7
gccaccatgggttctggacctagcaattttgtgaacaaagtagatggggccagcgcccccattaaggagcacgctatcccagctgccgcagcggatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 80 - protein, 113 aa, synthetic construct
  source 1..113: note = Mut 8
MGSGPSNFVNKVDGASAPIKEHAIPSLTSAAAAYLYTIVTAVILLVILWFLYRYYKDKKARKKKEDILLRLYGRGLNLSRLDPSVICSLGGSAPNLQHRGLERTEDKLVNPFI

SEQ ID NO: 81 - DNA, 348 bp, synthetic construct
  source 1..348: mol_type = other DNA; note = Mut 8
gccaccatgggttctggacctagcaattttgtgaacaaagtagatggggccagcgcccccattaaggagcacgctatcccatcactgacgagtgctgccgcagcgtatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 82 - protein, 113 aa, synthetic construct
  source 1..113: note = Mut 9
MGSGPSNFVNKVDGASAPIKEHAIPSLTSDLKDYLYTIVTAVILLVILWFLYRYYAAAAARKKKEDILLRLYGRGLNLSRLDPSVICSLGGSAPNLQHRGLERTEDKLVNPFI

SEQ ID NO: 83 - DNA, 348 bp, synthetic construct
  source 1..348: mol_type = other DNA; note = Mut 9
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacgctgccgcagcggcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 84 - protein, 113 aa, synthetic construct
  source 1..113: note = Mut 10
MGSGPSNFVNKVDGASAPIKEHAIPSLTSDLKDYLYTIVTAVILLVILWFLYRYYKDKKAAAAKEDILLRLYGRGLNLSRLDPSVICSLGGSAPNLQHRGLERTEDKLVNPFI

SEQ ID NO: 85 - DNA, 348 bp, synthetic construct
  source 1..348: mol_type = other DNA; note = Mut 10
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagctgccgcagcgaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 86 - protein, 113 aa, synthetic construct
  source 1..113: note = Mut 11
MGSGPSNFVNKVDGASAPIKEHAIPSLTSDLKDYLYTIVTAVILLVILWFLYRYYKDKKARKKAAAALLRLYGRGLNLSRLDPSVICSLGGSAPNLQHRGLERTEDKLVNPFI

SEQ ID NO: 87 - DNA, 348 bp, synthetic construct
  source 1..348: mol_type = other DNA; note = Mut 11
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaaggctgccgcagcgctgctcaggttgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 88 - protein, 113 aa, synthetic construct
  source 1..113: note = Mut 12
MGSGPSNFVNKVDGASAPIKEHAIPSLTSDLKDYLYTIVTAVILLVILWFLYRYYKDKKARKKKEDIAAAAYGRGLNLSRLDPSVICSLGGSAPNLQHRGLERTEDKLVNPFI

SEQ ID NO: 89 - DNA, 348 bp, synthetic construct
  source 1..348: mol_type = other DNA; note = Mut 12
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatagctgccgcagcgtacggtcgcggtctgaacctttctcgccttgatccttcagtaatctgcagcttgggaggctctgcacccaacctccaacacaggggacttgaacgaaccgaagataagctcgtaaaccctttcatataa

SEQ ID NO: 90 - protein, 113 aa, synthetic construct
  source 1..113: note = Mut 13
MGSGPSNFVNKVDGASAPIKEHAIPSLTSDLKDYLYTIVTAVILLVILWFLYRYYKDKKARKKKEDILLRLAAAALNLSRLDPSVICSLGGSAPNLQHRGLERTEDKLVNPFI

SEQ ID NO: 91 - DNA, 348 bp, synthetic construct
  source 1..348: mol_type = other DNA; note = Mut 13
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttggctgccgcagcgctgaacctttctcgccttgatccttcagtaatctgcagcttgggaggctctgcacccaacctccaacacaggggacttgaacgaaccgaagataagctcgtaaaccctttcatataa

SEQ ID NO: 92 - protein, 113 aa, synthetic construct
  source 1..113: note = Mut 14
MGSGPSNFVNKVDGASAPIKEHAIPSLTSDLKDYLYTIVTAVILLVILWFLYRYYKDKKARKKKEDILLRLYGRGAAAARLDPSVICSLGGSAPNLQHRGLERTEDKLVNPFI

SEQ ID NO: 93 - DNA, 348 bp, synthetic construct
  source 1..348: mol_type = other DNA; note = Mut 14
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtgctgccgcagcgcgccttgatccttcagtaatctgcagcttgggaggctctgcacccaacctccaacacaggggacttgaacgaaccgaagataagctcgtaaaccctttcatataa

SEQ ID NO: 94 - protein, 113 aa, synthetic construct
  source 1..113: note = Mut 15
MGSGPSNFVNKVDGASAPIKEHAIPSLTSDLKDYLYTIVTAVILLVILWFLYRYYKDKKARKKKEDILLRLYGRGLNLSAAAASVICSLGGSAPNLQHRGLERTEDKLVNPFI

SEQ ID NO: 95 - DNA, 348 bp, synthetic construct
  source 1..348: mol_type = other DNA; note = Mut 15
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctgctgccgcagcgtcagtaatctgcagcttgggaggctctgcacccaacctccaacacaggggacttgaacgaaccgaagataagctcgtaaaccctttcatataa

SEQ ID NO: 96 - protein, 113 aa, synthetic construct
  source 1..113: note = Mut 16
MGSGPSNFVNKVDGASAPIKEHAIPSLTSDLKDYLYTIVTAVILLVILWFLYRYYKDKKARKKKEDILLRLYGRGLNLSRLDPAAAASLGGSAPNLQHRGLERTEDKLVNPFI

SEQ ID NO: 97 - DNA, 348 bp, synthetic construct
  source 1..348: mol_type = other DNA; note = Mut 16
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatcctgctgccgcagcgagcttgggaggctctgcacccaacctccaacacaggggacttgaacgaaccgaagataagctcgtaaaccctttcatataa

SEQ ID NO: 98 - protein, 113 aa, synthetic construct
  source 1..113: note = Mut 17
MGSGPSNFVNKVDGASAPIKEHAIPSLTSDLKDYLYTIVTAVILLVILWFLYRYYKDKKARKKKEDILLRLYGRGLNLSRLDPSVICAAAASAPNLQHRGLERTEDKLVNPFI

SEQ ID NO: 99 - DNA, 348 bp, synthetic construct
  source 1..348: mol_type = other DNA; note = Mut 17
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcgctgccgcagcgtctgcacccaacctccaacacaggggacttgaacgaaccgaagataagctcgtaaaccctttcatataa

SEQ ID NO: 100 - protein, 113 aa, synthetic construct
  source 1..113: note = Mut 18
MGSGPSNFVNKVDGASAPIKEHAIPSLTSDLKDYLYTIVTAVILLVILWFLYRYYKDKKARKKKEDILLRLYGRGLNLSRLDPSVICSLGGAAAALQHRGLERTEDKLVNPFI

SEQ ID NO: 101 - DNA, 348 bp, synthetic construct
  source 1..348: mol_type = other DNA; note = Mut 18
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggcgctgccgcagcgctccaacacaggggacttgaacgaaccgaagataagctcgtaaaccctttcatataa

SEQ ID NO: 102 - protein, 113 aa, synthetic construct
  source 1..113: note = Mut 19
MGSGPSNFVNKVDGASAPIKEHAIPSLTSDLKDYLYTIVTAVILLVILWFLYRYYKDKKARKKKEDILLRLYGRGLNLSRLDPSVICSLGGSAPNAAAAGLERTEDKLVNPFI

SEQ ID NO: 103 - DNA, 348 bp, synthetic construct
  source 1..348: mol_type = other DNA; note = Mut 19
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacgctgccgcagcgggacttgaacgaaccgaagataagctcgtaaaccctttcatataa

SEQ ID NO: 104 - protein, 113 aa, synthetic construct
  source 1..113: note = Mut 20
MGSGPSNFVNKVDGASAPIKEHAIPSLTSDLKDYLYTIVTAVILLVILWFLYRYYKDKKARKKKEDILLRLYGRGLNLSRLDPSVICSLGGSAPNLQHRAAAATEDKLVNPFI

SEQ ID NO: 105 - DNA, 348 bp, synthetic construct
  source 1..348: mol_type = other DNA; note = Mut 20
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacagggctgccgcagcgaccgaagataagctcgtaaaccctttcatataa

SEQ ID NO: 106 - protein, 113 aa, synthetic construct; identical to SEQ ID NO: 3
  source 1..113: note = Mut 21

SEQ ID NO: 107 - DNA, 348 bp, synthetic construct
  source 1..348: mol_type = other DNA; note = Mut 21
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgagctgccgcagcgctcgtaaaccctttcatataa

SEQ ID NO: 108 - protein, 113 aa, synthetic construct; identical to SEQ ID NO: 3
  source 1..113: note = Mut 22

SEQ ID NO: 109 - DNA, 348 bp, synthetic construct
  source 1..348: mol_type = other DNA; note = Mut 22
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataaggctgccgcagcgttcatataa

SEQ ID NO: 110 - protein, 113 aa, synthetic construct; identical to SEQ ID NO: 3
  source 1..113: note = Mut 23

SEQ ID NO: 111 - DNA, 348 bp, synthetic construct
  source 1..348: mol_type = other DNA; note = Mut 23
gccaccatgggttctggacctagcaattttgtgaacaaagtagatggggccagcgcccccattaaggagcacgctatcccatcactgacgagtgatctgaaagactatctctatactatagtcacagcagttatccttctcgtgatcttgtggttcctctacagatattacaaggacaagaaagcaaggaaaaagaaggaggatatactgctcaggttgtacggtcgcggtctgaacctttctcgccttgatccttcagtaatctgcagcttgggaggctctgcacccaacctccaacacaggggacttgaacgaaccgaagataagctcgtaaaccctgctgcctaa

SEQ ID NO: 112 - protein, 113 aa, synthetic construct
  source 1..113: note = p13_Alanine_5/9
MGSGPSNFVNKVDGASAAAAAHAIPSLTSDLKDYLYTIVTAVILLVILWFLYRYYAAAAARKKKEDILLRLYGRGLNLSRLDPSVICSLGGSAPNLQHRGLERTEDKLVNPFI

SEQ ID NO: 113 - protein, 113 aa, synthetic construct
  source 1..113: note = p13_Alanine_5/17
MGSGPSNFVNKVDGASAAAAAHAIPSLTSDLKDYLYTIVTAVILLVILWFLYRYYKDKKARKKKEDILLRLYGRGLNLSRLDPSVICAAAASAPNLQHRGLERTEDKLVNPFI

SEQ ID NO: 114 - protein, 113 aa, synthetic construct
  source 1..113: note = p13_Alanine_5/18
MGSGPSNFVNKVDGASAAAAAHAIPSLTSDLKDYLYTIVTAVILLVILWFLYRYYKDKKARKKKEDILLRLYGRGLNLSRLDPSVICSLGGAAAALQHRGLERTEDKLVNPFI

SEQ ID NO: 115 - protein, 113 aa, synthetic construct
  source 1..113: note = p13_Alanine_5/20
MGSGPSNFVNKVDGASAAAAAHAIPSLTSDLKDYLYTIVTAVILLVILWFLYRYYKDKKARKKKEDILLRLYGRGLNLSRLDPSVICSLGGSAPNLQHRAAAATEDKLVNPFI

SEQ ID NO: 116 - protein, 113 aa, synthetic construct
  source 1..113: note = p13_Alanine_9/17
MGSGPSNFVNKVDGASAPIKEHAIPSLTSDLKDYLYTIVTAVILLVILWFLYRYYAAAAARKKKEDILLRLYGRGLNLSRLDPSVICAAAASAPNLQHRGLERTEDKLVNPFI

SEQ ID NO: 117 - protein, 113 aa, synthetic construct
  source 1..113: note = p13_Alanine_9/18
MGSGPSNFVNKVDGASAPIKEHAIPSLTSDLKDYLYTIVTAVILLVILWFLYRYYAAAAARKKKEDILLRLYGRGLNLSRLDPSVICSLGGAAAALQHRGLERTEDKLVNPFI

SEQ ID NO: 118 - protein, 113 aa, synthetic construct
  source 1..113: note = p13_Alanine_9/20
MGSGPSNFVNKVDGASAPIKEHAIPSLTSDLKDYLYTIVTAVILLVILWFLYRYYAAAAARKKKEDILLRLYGRGLNLSRLDPSVICSLGGSAPNLQHRAAAATEDKLVNPFI

SEQ ID NO: 119 - protein, 113 aa, synthetic construct
  source 1..113: note = p13_Alanine_17/18
MGSGPSNFVNKVDGASAPIKEHAIPSLTSDLKDYLYTIVTAVILLVILWFLYRYYKDKKARKKKEDILLRLYGRGLNLSRLDPSVICAAAAAAAALQHRGLERTEDKLVNPFI

SEQ ID NO: 120 - protein, 113 aa, synthetic construct
  source 1..113: note = p13_Alanine_17/20
MGSGPSNFVNKVDGASAPIKEHAIPSLTSDLKDYLYTIVTAVILLVILWFLYRYYKDKKARKKKEDILLRLYGRGLNLSRLDPSVICAAAASAPNLQHRAAAATEDKLVNPFI

SEQ ID NO: 121 - protein, 113 aa, synthetic construct
  source 1..113: note = p13_Alanine_18/20
MGSGPSNFVNKVDGASAPIKEHAIPSLTSDLKDYLYTIVTAVILLVILWFLYRYYKDKKARKKKEDILLRLYGRGLNLSRLDPSVICSLGGAAAALQHRAAAATEDKLVNPFI

SEQ ID NO: 122 - protein, 113 aa, synthetic construct
  source 1..113: note = p13_Alanine_5/9/17
MGSGPSNFVNKVDGASAAAAAHAIPSLTSDLKDYLYTIVTAVILLVILWFLYRYYAAAAARKKKEDILLRLYGRGLNLSRLDPSVICAAAASAPNLQHRGLERTEDKLVNPFI

SEQ ID NO: 123 - protein, 113 aa, synthetic construct
  source 1..113: note = p13_Alanine_5/9/18
MGSGPSNFVNKVDGASAAAAAHAIPSLTSDLKDYLYTIVTAVILLVILWFLYRYYAAAAARKKKEDILLRLYGRGLNLSRLDPSVICSLGGAAAALQHRGLERTEDKLVNPFI

SEQ ID NO: 124 - protein, 113 aa, synthetic construct
  source 1..113: note = p13_Alanine_5/9/20
MGSGPSNFVNKVDGASAAAAAHAIPSLTSDLKDYLYTIVTAVILLVILWFLYRYYAAAAARKKKEDILLRLYGRGLNLSRLDPSVICSLGGSAPNLQHRAAAATEDKLVNPFI

SEQ ID NO: 125 - protein, 113 aa, synthetic construct
  source 1..113: note = p13_Alanine_5/17/18
MGSGPSNFVNKVDGASAAAAAHAIPSLTSDLKDYLYTIVTAVILLVILWFLYRYYKDKKARKKKEDILLRLYGRGLNLSRLDPSVICAAAAAAAALQHRGLERTEDKLVNPFI

SEQ ID NO: 126 - protein, 113 aa, synthetic construct
  source 1..113: note = p13_Alanine_5/17/20
MGSGPSNFVNKVDGASAAAAAHAIPSLTSDLKDYLYTIVTAVILLVILWFLYRYYKDKKARKKKEDILLRLYGRGLNLSRLDPSVICAAAASAPNLQHRAAAATEDKLVNPFI

SEQ ID NO: 127 - protein, 113 aa, synthetic construct
  source 1..113: note = p13_Alanine_5/18/20
MGSGPSNFVNKVDGASAAAAAHAIPSLTSDLKDYLYTIVTAVILLVILWFLYRYYKDKKARKKKEDILLRLYGRGLNLSRLDPSVICSLGGAAAALQHRAAAATEDKLVNPFI

SEQ ID NO: 128 - protein, 113 aa, synthetic construct
  source 1..113: note = p13_Alanine_9/17/18
MGSGPSNFVNKVDGASAPIKEHAIPSLTSDLKDYLYTIVTAVILLVILWFLYRYYAAAAARKKKEDILLRLYGRGLNLSRLDPSVICAAAAAAAALQHRGLERTEDKLVNPFI

SEQ ID NO: 129 - protein, 113 aa, synthetic construct
  source 1..113: note = p13_Alanine_9/17/20
MGSGPSNFVNKVDGASAPIKEHAIPSLTSDLKDYLYTIVTAVILLVILWFLYRYYAAAAARKKKEDILLRLYGRGLNLSRLDPSVICAAAASAPNLQHRAAAATEDKLVNPFI

SEQ ID NO: 130 - protein, 113 aa, synthetic construct
  source 1..113: note = p13_Alanine_9/18/20
MGSGPSNFVNKVDGASAPIKEHAIPSLTSDLKDYLYTIVTAVILLVILWFLYRYYAAAAARKKKEDILLRLYGRGLNLSRLDPSVICSLGGAAAALQHRAAAATEDKLVNPFI

SEQ ID NO: 131 - protein, 113 aa, synthetic construct
  source 1..113: note = p13_Alanine_17/18/20
MGSGPSNFVNKVDGASAPIKEHAIPSLTSDLKDYLYTIVTAVILLVILWFLYRYYKDKKARKKKEDILLRLYGRGLNLSRLDPSVICAAAAAAAALQHRAAAATEDKLVNPFI

SEQ ID NO: 132 - protein, 113 aa, synthetic construct
  source 1..113: note = p13_Alanine_5/9/17/18
MGSGPSNFVNKVDGASAAAAAHAIPSLTSDLKDYLYTIVTAVILLVILWFLYRYYAAAAARKKKEDILLRLYGRGLNLSRLDPSVICAAAAAAAALQHRGLERTEDKLVNPFI

SEQ ID NO: 133 - protein, 113 aa, synthetic construct
  source 1..113: note = p13_Alanine_5/9/17/20
MGSGPSNFVNKVDGASAAAAAHAIPSLTSDLKDYLYTIVTAVILLVILWFLYRYYAAAAARKKKEDILLRLYGRGLNLSRLDPSVICAAAASAPNLQHRAAAATEDKLVNPFI

SEQ ID NO: 134 - protein, 113 aa, synthetic construct
  source 1..113: note = p13_Alanine_5/9/18/20
MGSGPSNFVNKVDGASAAAAAHAIPSLTSDLKDYLYTIVTAVILLVILWFLYRYYAAAAARKKKEDILLRLYGRGLNLSRLDPSVICSLGGAAAALQHRAAAATEDKLVNPFI

SEQ ID NO: 135 - protein, 113 aa, synthetic construct
  source 1..113: note = p13_Alanine_5/17/18/20
MGSGPSNFVNKVDGASAAAAAHAIPSLTSDLKDYLYTIVTAVILLVILWFLYRYYKDKKARKKKEDILLRLYGRGLNLSRLDPSVICAAAAAAAALQHRAAAATEDKLVNPFI

SEQ ID NO: 136 - protein, 113 aa, synthetic construct
  source 1..113: note = p13_Alanine_9/17/18/20
MGSGPSNFVNKVDGASAPIKEHAIPSLTSDLKDYLYTIVTAVILLVILWFLYRYYAAAAARKKKEDILLRLYGRGLNLSRLDPSVICAAAAAAAALQHRAAAATEDKLVNPFI

SEQ ID NO: 137 - protein, 113 aa, synthetic construct
  source 1..113: note = p13_Alanine_5/9/17/18/20
MGSGPSNFVNKVDGASAAAAAHAIPSLTSDLKDYLYTIVTAVILLVILWFLYRYYAAAAARKKKEDILLRLYGRGLNLSRLDPSVICAAAAAAAALQHRAAAATEDKLVNPFI

SEQ ID NO: 138 - protein, 122 aa, synthetic construct
  source 1..122: note = P13_HA_1
MGSGPSNFVNKVDYPYDVPDYAGASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 139 - protein, 122 aa, synthetic construct
  source 1..122: note = P13_HA_2
MGSGPSNFVNKVDGYPYDVPDYAASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 140 - protein, 122 aa, synthetic construct
  source 1..122: note = P13_HA_3
MGSGPSNFVNKVDGAYPYDVPDYASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 141 - protein, 122 aa, synthetic construct
  source 1..122: note = P13_HA_4
MGSGPSNFVNKVDGASYPYDVPDYAAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 142 - protein, 122 aa, synthetic construct
  source 1..122: note = P13_HA_5
MGSGPSNFVNKVDGASAYPYDVPDYAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 143 - protein, 122 aa, synthetic construct
  source 1..122: note = P13_HA_6
MGSGPSNFVNKVDGASAPYPYDVPDYAIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 144 - protein, 122 aa, synthetic construct
  source 1..122: note = P13_HA_7
MGSGPSNFVNKVDGASAPIYPYDVPDYAKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 145 - protein, 122 aa, synthetic construct
  source 1..122: note = P13_HA_8
MGSGPSNFVNKVDGASAPIKYPYDVPDYAEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 146 - protein, 122 aa, synthetic construct
  source 1..122: note = P13_HA_9
MGSGPSNFVNKVDGASAPIKEYPYDVPDYAHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 147 - protein, 122 aa, synthetic construct
  source 1..122: note = P13_HA_10
MGSGPSNFVNKVDGASAPIKEHYPYDVPDYAAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 148 - protein, 122 aa, synthetic construct
  source 1..122: note = P13_HA_11
MGSGPSNFVNKVDGASAPIKEHAYPYDVPDYAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 149 - protein, 122 aa, synthetic construct
  source 1..122: note = P13_HA_12
MGSGPSNFVNKVDGASAPIKEHAIYPYDVPDYAPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 150 - protein, 338 aa, synthetic construct
  source 1..338: note = Anti-HA scfv (CD8a leading peptide / anti-HA scfv(1) / CD8a transmembrane domain)
MALPVTALLLPLALLLHAARPAEVKLVESGGGLVKPGGSLKLSCAASGFTFSSYGMSWVRQTPEKRLEWVATISRGGSYTYYPDSVKGRFTISRDNAKNTLYLQMSSLRSEDTAIYYCARRETYDEKGFAYWGQGTTLTVSSGGGGSGGGGSGGGGSDIVLTQSPASLTVSLGQRATISCKSSQSLLNSGNQKNYLTWYQQKPGQPPKLLIYWASTRESGIPARFSGSGSGTDFTLNIHPVEEEDAATYYCQNDNSHPLTFGAGTKLEITTTPAPRPPTPAPTIASQPLSLRPEACRPAAGGAVHTRGLDFACDIYIWAPLAGTCGVLLLSLVITLYC

SEQ ID NO: 151 - protein, 113 aa, synthetic construct
  source 1..113: note = Mut 2
MGSGPAAAANKVDGASAPIKEHAIPSLTSDLKDYLYTIVTAVILLVILWFLYRYYKDKKARKKKEDILLRLYGRGLNLSRLDPSVICSLGGSAPNLQHRGLERTEDKLVNPFI

SEQ ID NO: 152 - protein, 125 aa, Veiled Chameleon
  source 1..125: note = Veiled Chameleon (VC)
MGSGPSNFVNHAPGEAIVTGLEKGADKVAGTISHTIWEVIAGLVALLIFLAIGFWLFKHLQKRRERARQLSEFQKRYLRNSYRLSEVQRPISLHEYEDPYEPPSRKKAPPPPYSTYVNIDDVSAV

(19) United States
(12) Patent Application Publication
(10) Pub. No.: US 2024/0293462 A1
(43) Pub. Date: Sep. 5, 2024

(54) Immune Cell Fusion (ICF) and Uses Thereof

(71) Applicant: Trustees of Boston University, Boston, MA (US)

(72) Inventors: Michael Howard Raymond, Dorchester, MA (US); Ahmad S. Khalil, Lexington, MA (US)

(21) Appl. No.: 18/593,853

(22) Filed: Mar. 1, 2024

Related U.S. Application Data
(60) Provisional application No. 63/488,382, filed on Mar. 3, 2023.
(60) Provisional application No. 63/583,802, filed on Sep. 19, 2023.

(51) Int. Cl. A61K 35/17; A61K 39/00; A61P 35/00; C07K 14/005; C07K 16/28; C12N 5/0783; C12N 15/10

(52) U.S. Cl. CPC A61K 35/17; A61K 39/4611; A61K 39/4632; A61K 39/464412; A61P 35/00; C07K 14/005; C07K 16/28; C12N 5/0636; C12N 15/1037; C12N 2720/12222; C12N 2720/12232

(57) ABSTRACT

Provided herein, in various embodiments, are mammalian cells (e.g., immune effector cells) comprising a nucleotide sequence encoding an exogenous fusogen. Also provided herein, in various embodiments, are methods of treating cancer in a subject in need thereof, comprising administering to the subject mammalian cells (e.g., immune effector cells) disclosed herein. Also provided herein, in various embodiments, are methods of killing a cancer cell, comprising contacting the cancer cell with mammalian cells (e.g., immune effector cells) disclosed herein.

### RELATED APPLICATIONS

This application claims the benefit of U.S. Provisional Application No. 63/488,382, filed on Mar. 3, 2023, and U.S. Provisional Application No. 63/583,802, filed on Sep. 19, 2023. The entire teachings of the above applications are incorporated herein by reference.

### GOVERNMENT SUPPORT

This invention was made with government support under Grant Number EB029483 from the National Institutes of Health. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 62002004-002 Sequence Listing.xml; created Mar. 1, 2024, 180,341 Bytes in size.

### BACKGROUND

CAR-T therapies have reshaped cancer treatment; however, currently available therapies suffer from inefficient tumor ablation. As a result, most patients treated with CAR-T exhibit tumor relapse and often require additional interventions to ameliorate symptoms. For example, lymphoma is a devastating disease that afflicts around 60,000 people per year. CAR-T treatment has resulted in rare instances of complete remission for certain B-cell lymphomas; however, most patients present with tumor reoccurrence or resistance. Pre-clinical treatment strategies for treatment of B-cell lymphoma have encompassed use of immunostimulatory agents to increase immune response and promote durable tumor regression. These strategies lack targetability and control, which limits clinical viability.

### SUMMARY

There is a need to develop a new class of cell-based immunotherapy which unlocks on-demand tumor ablation through targeted immune effector cell (e.g., T cell)-tumor cell fusion. Immune Cell Fusion (ICF) harnesses synthetic receptors and genetic circuits and eliminates cancer cells (e.g., B-cell lymphoma and lung cancer cells) through user-defined cell death programs via targeted ICF tumor cell fusion.

Provided herein, in some embodiments, are mammalian cells comprising a nucleotide sequence encoding an exogenous fusogen, wherein the mammalian cell:
a) is an immune effector cell;
b) further comprises a nucleotide sequence encoding a cell-targeting molecule, or both a) and b).

Also provided herein, in some embodiments, are immune effector cells comprising a nucleotide sequence encoding an exogenous fusogen.

In some embodiments, an immune effector cell is a T cell, a monocyte, a B cell, a natural killer (NK) cell, a mononuclear phagocytic cell (e.g., a macrophage), or a dendritic cell. In some embodiments, an immune effector cell comprises a nucleotide sequence encoding a cell-targeting molecule.

Also provided herein are mammalian cells (e.g., immune effector cells) comprising a nucleotide sequence encoding a cell-targeting molecule and a nucleotide sequence encoding an exogenous fusogen.

In some embodiments, an exogenous fusogen is an engineered fusogen.

In some embodiments, an exogenous fusogen is a viral fusogen.

In some embodiments, a fusogen is derived from an avian orthoreovirus (ARV; also referred to herein as ARVch), a Nelson Bay (NB) virus, a Broome orthoreovirus (BroV), a reptilian orthoreovirus (RRV), a Baboon orthoreovirus (BRV), a group C aquareovirus (AqRV-C), a group A aquareovirus (AqRV-A), a halibut reovirus, a Mahlapitisi orthoreovirus (MaRV), a Muscovy duck orthoreovirus (MdRV), a reptilian Testudine orthoreovirus (RRVtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaceous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), or a human rotavirus species B NSP-1 (HuRVB), or a variant thereof.

In some embodiments, a fusogen is derived from an avian orthoreovirus (ARV), a Nelson Bay (NB) virus (also known as Nelson Bay orthoreovirus), a Broome orthoreovirus (BroV), a reptilian orthoreovirus (RRV), a Baboon orthoreovirus (BRV), a group C aquareovirus (AqRV-C), a group A aquarcovirus (AqRV-A), a halibut reovirus, a Mahlapitisi orthoreovirus (MaRV), a Muscovy duck orthoreovirus (MdRV), a reptilian Testudine orthoreovirus (RRVtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaccous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), a human rotavirus species B NSP-1 (HuRVB), or a veiled chameleon reovirus (VC), or a variant thereof.

In some embodiments, an exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof, or any combination of the foregoing.

In some embodiments, an exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
or any combination of the foregoing.

In some embodiments, a cell-targeting molecule recognizes a cell-surface marker for cancer cells, neurons, oligodendrocytes, microglia, neural stem cells, hematopoietic stem cells, and/or pancreatic beta cells. In some embodiments, a cell-targeting molecule comprises a B-cell lymphoma-derived antigen. In some embodiments, a cell-targeting molecule comprises a lung cancer-derived antigen.

Also provided herein are compositions (e.g., pharmaceutical compositions) and kits comprising the mammalian cells (e.g., immune effector cells) disclosed herein.

Also provided herein are methods of treating cancer in a subject in need thereof, comprising administering to the subject a therapeutically effective amount of the mammalian cells (e.g., immune effector cells), compositions (e.g., pharmaceutical compositions), or kits, e.g., those disclosed herein.

Also provided herein are mammalian cells (e.g., immune effector cells), compositions (e.g., pharmaceutical compositions), or kits, e.g., those disclosed herein, for use in treating cancer in a subject in need thereof.

Also provided herein are uses of mammalian cells (e.g., immune effector cells), compositions (e.g., pharmaceutical compositions), or kits, e.g., those disclosed herein, for the preparation of a medicament for treating cancer in a subject in need thereof.

Also provided herein are methods of killing a cancer cell, comprising contacting the cancer cell with an effective dosage of the mammalian cells (e.g., immune effector cells), compositions (e.g., pharmaceutical compositions), or kits, e.g., those disclosed herein.

Also provided herein are methods of screening a library of antigens. In some embodiments, provided herein are methods of screening a library of peptide major histocompatibility complex (pMHC) antigens expressed on the surfaces of a population of mammalian cells, comprising:
a) co-culturing the population of mammalian cells with a population of fusogen-expressing T cells,
b) identifying a fused cell produced by fusion of a cell from the population of mammalian cells and a cell from the population of T cells, and
c) identifying the pMHC antigen expressed on the surface of the fused cell.

In some embodiments, a pMHC antigen is a tumor antigen, a pathogenic antigen, a bacterial antigen, or a viral antigen.

Also provided herein are methods of screening a library of fusogens useful for mediating cell fusion, comprising:
a) expressing the library of fusogens in a population of mammalian cells, wherein each mammalian cell expresses a unique fusogen;
b) co-culturing the population of mammalian cells with a population of target cells,
c) identifying a fused cell produced by fusion of a cell of the population of mammalian cells and a cell of the population of target cells, and
d) identifying the exogenous fusogen expressed in the fused cell.

Also provided herein are fused cells generated by fusion between a target cell and a mammalian cell (e.g., an immune effector cell) disclosed herein.

Also provided herein are methods of fusing a target cell and a mammalian cell (e.g., an immune effector cell), comprising co-culturing the target cell with a mammalian cell (e.g., an immune effector cell) disclosed herein.

Also provided herein are fusogens comprising:
a) an extracellular domain of a viral fusogen derived from a BRV, a BroV, an ARV, an AqRV-A, a RRV, or a variant thereof,
b) a transmembrane domain of a viral fusogen derived from a PsRV, a MdRV, an ARV, a RRV, or a variant thereof,
c) an intracellular domain of a viral fusogen derived from a RRV, an AqRV-C, a MdRV, a Nelson Bay virus, a MaRV, an ARVch, or a variant thereof,
or any combination of the foregoing.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 illustrates Immune Cell Fusion (ICF), which leverages immune effector cell (e.g., T cell) endogenous homing and recognition to fuse to and, in some embodiments, ablate a target cell (e.g., a tumor cell).

FIG. 2A shows cell-cell fusion between 293T target cells and FAST fusogen-expressing 293T cells. The number of fusion events per field of view (y-axis) is shown for different fusogens compared to a control (x-axis). FIG. 2B shows cell-cell fusion between 293T target cells and mammalian fusogen-expressing 293T cells (HERV-Pb1=human endogenous retrovirus envelope fusion protein; SynA=murine syncytin-A fusion protein). FIG. 2C shows cell-cell fusion between 293T target cells and FAST fusogen-expressing human T cells. The fusion index (number of fused nuclei per field of view) (y-axis) is shown for different fusogens compared to a control (x-axis).

FIG. 3A shows that ICF fusion is enhanced via antibody binding. FIG. 3B shows that antigen-directed ICF T cells exhibit low off-target fusion. Asterisks indicate statistical significance (Two-Way ANOVA; *: p<0.0332, **: p<0.0021, ***: p<0.0002). FIG. 3C shows that adhesion augments targeted ICF fusion. It illustrates targeted ICF fusion (diagram, left) and shows that adhesion augments targeted ICF fusion (right). FIG. 3D is an enlarged inset of a diagram of FIG. 3C and illustrates binding of an anti-CD19 scFv, expressed on the surface of an ICF T cell, to a CD19 antigen expressed on the surface of a target cell.

FIG. 4 illustrates intracellular signaling mechanisms underlying p13 FAST fusogen initiated cell-cell fusion (left) and shows the effects of inhibitors of each of these mechanisms (right).

FIG. 5 shows effects of mutating various regions of p13 on p13-mediated cell-cell fusion.

FIGS. 6A-6C show an example high-throughput fusion assay involving an inducible (e.g., tetracycline on) reporter system. FIG. 6A illustrates fusion of an ICF T-cell with a CD19⁺ target cell resulting in induction of reporter (e.g., mCherry) expression via reverse tetracycline-controlled transactivator (rtTA) in the fused cell. FIG. 6B illustrates doxycycline (DOX)-inducible, rtTA-mediated fusogen expression controlled by a Tet response element (TRE) and synthetic YB_TATA promoter (pybTATA). Expression of rtTA is driven by a strong constitutive promoter such as human phosphoglycerate kinase promoter (hPGK). FIG. 6C illustrates DOX-inducible, rtTA-mediated expression of an mCherry reporter. The presence of DOX, rtTA, and the inducible mCherry expression cassette in the fused cell act as a fusion-specific reporter. Together, FIGS. 6A-C illustrate example controls over fusion potential (e.g., inducible fusogen expression) and reporters (e.g., mCherry, RFP), that can be implemented individually or in combination and be adapted for various purposes.

FIG. 7 illustrates inducible forms of programmed cell death pathways: apoptosis, pyroptosis, and necroptosis (top); and shows their effects in Jurkat T cells (bottom). Cell viability was determined via Annexin V (phosphatidylserine, non-lytic death) and 7-AAD (membrane permeability, lytic death) staining at indicated time points (2-8 hours) after pathway (circuit) activation.

FIG. 8A illustrates SynNotch T-cell interaction with a CD19+B-cell. FIG. 8B illustrates CD19-dependent synthetic receptor activation upon co-culture of anti-CD19 SynNotch T cells (ICF T cells), which express anti-CD19 scFv, with CD19 antigen-expressing human B-cells. Red fluorescent protein (RFP) expression indicates receptor activation, e.g., binding of CD19 to anti-CD19 scFv and subsequent release of a transcription factor (TF) that drives expression of a reporter (e.g., RFP). FIG. 8C shows an assessment of fusion of ICF T cells, with or without anti-CD19 scFv, with CD19+ cells following co-culture. FIG. 8D illustrates fusion of ICF T cells and CD19+ cells with or without anti-CD19 scFv. FIG. 8E shows results of fusion, as illustrated in FIG. 8D, facilitated by fusogens ARV, NB, BroV, and RRV (x-axis). Open bars=ICF T cells lacking anti-CD19 scFv expression; patterned bars=ICF T cells with anti-CD19 scFv expression.

FIG. 9 shows effects of combining two to five selected p13 alanine mutations of FIG. 5 on p13-mediated cell-cell fusion.

FIG. 10A illustrates insertion of an HA epitope tag into the extracellular domain of p13. FIG. 10B shows results of an HA insertion scan at 12 sequential positions in the extracellular loop.

FIG. 11A illustrates fusion between ICF T cells expressing HA-tagged p13 (HA-p13) and CD19+ cancer/target cells. The top panel illustrates conditions in which the ICF T cells do or do not co-express HA-p13 and/or anti-HA tag scFv. The bottom panel illustrates conditions in which the CD19+ cancer/target cells express anti-HA tag scFv. FIG. 11B illustrates co-expression of HA-tagged p13 and anti-HA scFv on the surface of an ICF T-cell. FIG. 11C shows differences in fusion between ICF T cells (expressing wild-type p13 (p13), p13 and anti-HA scFv, HA-tagged p13 (p13 Site 7), or both HA-tagged p13 and anti-HA scFv) and CD19⁺ target cells expressing (right of dashed line) or not expressing (left of dashed line) anti-HA scFv. Asterisks indicate statistical significance.

FIGS. 12A-12C show that T cell-CD19⁺ target cell fusion is inhibited when treated with actin polymerization inhibitor Cytochalasin D (CytoD). FIG. 12A illustrates the relative location of Cytochalasin D activity in the cell. FIG. 12B is an enlarged inset of FIG. 12A and illustrates Cytochalasin D (Cytochalasin). FIG. 12C shows the effects of CytoD treatment on fusion involving ICF cells expressing BroV or RRV fusogens. FIGS. 12D-12F show that T cell-CD19⁺ target cell fusion is inhibited when treated with microtubule polymerization inhibitor Colchicine (Colchi). FIG. 12D illustrates the relative location of Colchicine activity in the cell. FIG. 12E is an enlarged inset of FIG. 12D and illustrates Colchicine. FIG. 12F shows the effects of Colchicine on fusion involving ICF cells expressing BroV or RRV fusogens. Asterisks indicate statistical significance.

### DETAILED DESCRIPTION

A description of example embodiments follows.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different. For example, unless the context clearly indicates otherwise, “a polypeptide” includes a single polypeptide, and two or more polypeptides.

Throughout this specification and the claims which follow, unless the context requires otherwise, the term “comprise,” and variations such as “comprises” and “comprising,” will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, the term “consisting of” excludes any element, step, or ingredient not specified in the claim element. When used herein, the term “consisting essentially of” does not exclude materials or steps that do not materially affect the basic and novel characteristics of the claim.

Also provided herein are corresponding embodiments for each and every embodiment featuring the term “comprising,” “containing,” “including,” or “having,” wherein those terms are replaced by the term “consisting of” and/or “consisting essentially of.”

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” “fewer than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the term “about” means within an acceptable error range for a particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of +20%, e.g., +10%, +5% or +1% of a given value. It is to be understood that the term “about” can precede any particular value specified herein, except for particular values used in the Exemplification. When “about” precedes a range, as in “90-99.9%,” the term “about” should be read as applying to both given values of the range, such that “about 90-99.9%” means about 90% to about 99.9%.

As used herein, a “polynucleotide” is defined as a plurality of nucleotides and/or nucleotide analogs linked together in a single molecule. In some embodiments, a polynucleotide disclosed herein comprises deoxyribonucleotides. In some embodiments, the polynucleotide comprises ribonucleotides. Non-limiting examples of polynucleotides include single-, double-, or multi-stranded deoxyribonucleic acid (DNA) or ribonucleic acid (RNA), DNA-RNA hybrids (e.g., each “T” position may be independently substituted by a “U” or vice versa), or a polymer comprising purine and pyrimidine bases, or other natural, chemically, or biochemically modified, non-natural, or derivatized nucleotide bases. The backbone of the polynucleotide can comprise sugars and phosphate groups, modified or substituted sugar or phosphate groups, a polymer of synthetic subunits such as phosphoramidates, or a combination thereof.

As used herein, the term “encoding” refers to specific sequences of nucleotides in a polynucleotide, such as a DNA (e.g., a cDNA) or an RNA (e.g., an mRNA), to serve as a template for synthesis of a protein having defined sequences of amino acids. Unless otherwise specified, a polynucleotide encoding an amino acid sequence can have any one nucleic acid sequence of all nucleic acid sequences that are degenerate versions of each other and that encode the amino acid sequence.

A protein or polypeptide disclosed or described herein (e.g., fusogen, target molecule, or payload) can comprise any suitable L- and/or D-amino acid, for example, common α-amino acids (e.g., alanine, glycine, valine), non-α-amino acids (e.g., ß-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide, or polypeptide can also be derivatized (e.g., alkylated) or labeled (e.g., with a detectable label, such as a fluorogen or a hapten) using methods known in the art. A protein or polypeptide disclosed or described herein (e.g., fusogen, target molecule, or payload) can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide, or polypeptide can be an analog of a known and/or naturally occurring peptide, for example, a peptide analog having one or more conservative amino acid residue substitutions.

As used herein, the term “sequence identity” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health (Altschul et al., 1990).

In some embodiments, an amino acid substitution is a conservative substitution. The term “conservative amino acid substitution(s)” or “conservative substitution(s)” refers to an amino acid substitution having a value of 0 or greater in BLOSUM62.

In some embodiments, an amino acid substitution is a highly conservative substitution. The term “highly conservative amino acid substitution(s)” or “highly conservative substitution(s)” refers to an amino acid substitution having a value of at least 1 (e.g., at least 2) in BLOSUM62.

As used herein, the term “antigen” refers to any substance that can be recognized by the immune system. “Antigen” broadly encompasses proteins, such as enzymes; peptides, such as polypeptides; carbohydrates, such as polysaccharides; haptens; nucleic acids, such as polynucleotides; and grafts. An antigen can be a self-antigen, an antigen produced by the body under normal conditions or as part of a disorder, a foreign antigen, or a non-self-antigen. Examples of self-antigens include self-antigens associated with cancers.

As used herein, the term “tumor-associated antigen” or “TAA” refers to a protein or polypeptide antigen that is expressed by a cancer cell (e.g., a tumor cell). For example, a TAA may be one or more surface proteins or polypeptides, nuclear proteins or glycoproteins, or fragments thereof, of a cancer cell (e.g., a tumor cell).

Cancer cells may be, for example, cells of a solid tumor (e.g., a tumor of the breast, lung, prostate, colon, bladder, ovary, kidney, stomach, colon, rectum, testes, head and/or neck, pancreas, brain, skin) or a hematologic cancer (e.g., leukemia, lymphoma, myeloma). As used herein, the term “cancer” may also refer to non-malignant hyperplasia, neoplasia, or pre-cancerous cells or lesions.

As used herein, the term “antibodies” refers to “full length antibodies” comprising two heavy (H) chains and two light (L) chains interconnected by disulfide bonds as well as multimers thereof (e.g., IgM). Each heavy chain comprises a heavy chain variable region (V_{H}) and a heavy chain constant region (comprising domains C_{H}1, hinge, C_{H}2, and C_{H}3). Each light chain comprises a light chain variable region (V_{L}) and a light chain constant region (C_{L}). The V_{H} and the V_{L} regions may be further subdivided into regions of hypervariability, termed complementarity determining regions (CDRs), interspersed with framework regions (FRs). Each V_{H} and V_{L} composes three CDRs and four FRs, arranged from amino-terminus to carboxy-terminus in the following order: FR1, CDR1, FR2, CDR2, FR3, CDR3, and FR4.

In some embodiments, an antibody is monospecific. In some embodiments, an antibody is multi-specific (e.g., bispecific). In some embodiments, an antibody is a monoclonal antibody (e.g., a murine, human, humanized, or chimeric antibody).

As used herein, the term “antigen-binding fragment” refers to a portion of an immunoglobulin molecule (e.g., antibody) that retains the antigen binding properties of the parental immunoglobulin molecule (e.g., full-length antibody). Non-limiting examples of antigen-binding fragments include an antibody heavy chain variable region (V_{H} region), an antibody light chain variable region (V_{L} region), an Fab fragment, an Fab′ fragment, an F(ab′)2 fragment, an Fd fragment, an Fv fragment, disulfide-linked Fvs (sdFv, e.g., diabody, triabody or tetrabody), a single-chain variable fragment (scFv, e.g., comprising an antibody heavy chain variable region (V_{H} region) and an antibody light chain variable region (V_{L} region) in either orientation), a domain antibody (dAb) consisting of one V_{H} domain or one V_{L} domain, a small modular immunopharmaceutical (SMIP), and rlgG.

In some embodiments, an antigen binding domain is an scFv. In some embodiments, an scFv comprises a humanized V_{H} region, a humanized V_{L} region, or both. In some embodiments, an scFv comprises a fully human V_{H} region, a fully human V_{L} region, or both. In some embodiments, a V_{H} region and a V_{L} region of an scFv are linked via a peptide linker. In some embodiments, a peptide linker has stretches of hydrophilic residues, for example, glycine and/or serine to increase flexibility, and glutamate and/or lysine to increase solubility. In some embodiments, an antigen binding domain comprises a heavy-chain antibody (comprising two or more heavy chains but lacking light chain), or an antigen-binding fragment thereof. Non-limiting examples of heavy chain antibodies include camelid Vhh (also referred to as VHH or V_{H}H) antibodies. Camelid antibodies are antibodies from the Camelidae family of mammals that include llamas, camels, and alpacas.

As used herein, the term “expression vector” refers to a replicable nucleic acid from which one or more proteins can be expressed when the expression vector is transformed into a suitable expression host cell. As used herein, the term “promoter” refers to a region of DNA to which RNA polymerase binds and initiates the transcription of a gene. As used herein, the term “operably linked” means that the nucleic acid is positioned in the recombinant polynucleotide, e.g., vector, in such a way that enables expression of the nucleic acid under control of the element (e.g., promoter) to which it is linked. As used herein, the term “selectable marker element” is an element that confers a trait suitable for artificial selection. Selectable marker elements can be negative or positive selection markers.

As used herein, the term “ex vivo” refers to methods conducted within or on cells or tissue in an artificial environment outside an organism with minimum alteration of natural conditions. As used herein, the term “in vivo” refers to a method that is conducted within living organisms in their normal, intact state. As used herein, the term “in vitro” method is conducted using components of an organism that have been isolated from its usual biological context.

As used herein, the term “fusion protein” refers to a synthetic, semi-synthetic or recombinant single protein molecule. A fusion protein can comprise all or a portion of two or more different proteins and/or polypeptides that are attached by covalent bonds (e.g., peptide bonds).

As used herein, the term “a subject” or “a patient” refers to a mammal (e.g., a human). In some embodiments, a subject is a mammal. In some embodiments, a subject is a mammal selected from a dog, a cat, a mouse, a rat, a hamster, a guinea pig, a horse, a pig, a sheep, a cow, a chimpanzee, a macaque, a cynomolgus, and a human. In some embodiments, a subject is a primate. In some embodiments, a subject is a human.

The term “a subject in need thereof” or “a patient in need thereof” refers to an animal (e.g., a mammal, such as a human), diagnosed with or suspected of having a disease or condition (e.g., a cancer), or one at risk of developing such a condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. In some embodiments, a pharmaceutical composition suitable for use in methods disclosed herein further comprises one or more pharmaceutically acceptable carriers.

The phrase “pharmaceutically acceptable” means that the substance or composition the phrase modifies is, within the scope of sound medical judgment, suitable for use in contact with the tissues of humans and lower animals without undue toxicity, irritation, allergic response, and the like, and are commensurate with a reasonable benefit/risk ratio.

“Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polynucleotide) is administered. Such vehicles may be liquids, such as water and oils, including those of petroleum, animal, vegetable, or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the agent in such pharmaceutical formulation may vary widely, i.e., from less than about 0.5% to at least about 1%, or to as much as 15% or 20%, 25%, 30%, 35%, 40%, 45%, or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

Non-limiting examples of pharmaceutically acceptable carriers are solvents, dispersion media, coatings, antibacterial and antifungal agents, isotonic and absorption-delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers are acetic acid, citric acid, formic acid, succinic acid, phosphoric acid, carbonic acid, malic acid, aspartic acid, histidine, boric acid, Tris buffers, HEPPSO, and HEPES.

Non-limiting examples of antioxidants are ascorbic acid, methionine, cysteine hydrochloride, sodium bisulfate, sodium metabisulfite, sodium sulfite, lecithin, citric acid, ethylenediamine tetraacetic acid (EDTA), sorbitol, and tartaric acid.

Non-limiting examples of amino acids are histidine, isoleucine, methionine, glycine, arginine, lysine, L-leucine, tri-leucine, alanine, glutamic acid, L-threonine, and 2-phenylamine.

Non-limiting examples of surfactants are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl-, or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl- or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl, and the like), benzalkonium chloride, benzethonium chloride, sodium dehydroacetate, thimerosal, or mixtures thereof.

Non-limiting examples of saccharides are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, or iso-maltulose.

Non-limiting examples of salts are acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids, such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like. In some embodiments, the salt is sodium chloride (NaCl).

As used herein, the term “pharmaceutically acceptable salt” refers to those salts which are, within the scope of sound medical judgment, suitable for use in contact with the tissues of mammals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the agents/compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Administering” or “administration,” as used herein, refers to providing a composition (e.g., a pharmaceutical composition) to a subject in need of treatment or prevention. Administering can be performed, for example, once, a plurality of times, and/or over one or more extended periods. Administration includes both direct administration (including self-administration), and indirect administration (including an act of prescribing a drug or directing a subject to consume an agent). For example, as used herein, one (e.g., a physician) who instructs a subject (e.g., a human patient) to self-administer a pharmaceutical composition, or to have a pharmaceutical composition administered by another and/or who provides a patient with a prescription for a pharmaceutical composition is administering an agent to a subject.

As used herein, the term “a therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of a pharmaceutical composition to be administered can be determined by a clinician of ordinary skill using the guidance provided herein and other methods known in the art. Relevant factors include the given agent (e.g., immune effector cells), the pharmaceutical formulation, the route of administration, the type of disease (e.g., cancer), the identity of the subject (e.g., age, sex, weight) or host being treated, and the like. Determining a dosage for a particular agent, subject and disease is well within the abilities of one of skill in the art. Preferably, a dosage does not cause or produces minimal adverse side effects.

Desired response or desired results include effects at the cellular level, tissue level, or clinical results. As such, “a therapeutically effective amount” or synonym thereto depends upon the context in which it is being applied. For example, in some embodiments, it is an amount of a composition sufficient to achieve a treatment response as compared to the response obtained without administration of the composition. In some embodiments, it is an amount that results in a beneficial or desired result in a subject as compared to a control. As defined herein, a therapeutically effective amount of a composition may be readily determined by one of ordinary skill by routine methods known in the art. Dosage regimen and route of administration may be adjusted to provide an optimum therapeutic response.

As used herein, the term “treating,” or its equivalents (e.g., “treatment” or “treat”), refers to the medical management of a subject with the intent to improve, ameliorate, stabilize (i.e., not worsen), prevent or cure a disease, pathological condition, or disorder, such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of a disease or condition (e.g., a cancer such as a solid tumor); preventing spread of the disease or condition; delay or slowing the progress of the disease or condition; amelioration or palliation of the disease or condition; and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment.

As used herein, a “vector” refers to a nucleic acid molecule which may be employed to introduce a nucleic acid sequence or gene into a cell, either in vitro, ex vivo, or in vivo.

As used herein, a “host” cell refers to a cell into which a polynucleotide has been introduced by molecular biology techniques. All techniques by which a polynucleotide can be introduced into a host cell, including transfection with viral vectors, transformation with plasmid vectors, and introduction of naked DNA by electroporation, lipofection, and particle gun acceleration are contemplated herein.

### Immune Effector Cells

As used herein, the term “immune effector cell” refers to a cell that is involved in an immune response, such as an immune effector response.

In some embodiments, an immune effector cell is a T cell, a monocyte, a B cell, a natural killer (NK) cell, a mononuclear phagocytic cell (e.g., a macrophage), or a dendritic cell.

In some embodiments, an immune effector cell is a T cell. In some embodiments, an immune effector cell is a helper CD4⁺ T cell, a cytotoxic CD8⁺ T cell, a memory T cell, a regulatory CD4⁺ T cell, an innate-like T cell, a natural killer T cell, a mucosal associated invariant T cell, or a Gamma Delta T cell. In some embodiments, an immune effector cell is a helper CD4⁺ T cell, a cytotoxic CD8⁺ T cell, a memory T cell, or a regulatory CD4⁺ T cell. In some embodiments, an immune effector cell is a helper CD4⁺ T cell. In some embodiments, an immune effector cell is a cytotoxic CD8⁺ T cell. In some embodiments, an immune effector cell is a memory T cell. In some embodiments, an immune effector cell is a regulatory CD4⁺ T cell.

In some embodiments, an immune effector cell is a monocyte. In some embodiments, an immune effector cell is a CD14⁺⁺CD16⁻ monocyte, a CD14⁺CD16⁺⁺ monocyte, or a CD14⁺⁺ CD16⁺ monocyte. In some embodiments, an immune effector cell is a CD14⁺+CD16 monocyte. In some embodiments, an immune effector cell is a CD14⁺⁺CD16⁺⁺ monocyte. In some embodiments, an immune effector cell is a CD14⁺+CD16⁺ monocyte.

In some embodiments, an immune effector cell is a B cell. In some embodiments, an immune effector cell is a plasmablast, a plasma cell, a lymphoplasmacytoid cell, a memory B cell, a B-2 cell, a B-1 cell, or a regulatory B cell (Breg). In some embodiments, an immune effector cell is a plasmablast. In some embodiments, an immune effector cell is a plasma cell. In some embodiments, an immune effector cell is a lymphoplasmacytoid cell. In some embodiments, an immune effector cell is a memory B cell. In some embodiments, an immune effector cell is a B-2 cell. In some embodiments, an immune effector cell is a B-1 cell. In some embodiments, an immune effector cell is a Breg.

In some embodiments, an immune effector cell is a NK cell. In some embodiments, an immune effector cell is CD56^{BRIGHT}. In some embodiments, an immune effector cell is CD56^{DIM}.

In some embodiments, an immune effector cell is a macrophage. In some embodiments, an immune effector cell is a mononuclear phagocytic cell or a mononuclear phagocyte system cell. In some embodiments, an immune effector cell is a mononuclear phagocytic cell. In some embodiments, an immune effector cell is an adipose tissue macrophage, a monocyte, a Kupffer cell, a sinus histiocyte, an alveolar macrophage (dust cell), a tissue macrophage (histiocyte; optionally leading to a giant cell), a microglial cell, a Hofbauer cell, an intraglomerular mesangial cell, an osteoclast, a Langerhans cell, an epithelioid cell, a red pulp macrophage (sinusoidal lining cell), a peritoneal macrophage, a lysomac, or a perivascular macrophage. In some embodiments, an immune effector cell is an adipose tissue macrophage. In some embodiments, an immune effector cell is a monocyte. In some embodiments, an immune effector cell is a Kupffer cell. In some embodiments, an immune effector cell is a sinus histiocyte. In some embodiments, an immune effector cell is an alveolar macrophage. In some embodiments, an immune effector cell is a tissue macrophage. In some embodiments, an immune effector cell is a microglial cell. In some embodiments, an immune effector cell is a Hofbauer cell. In some embodiments, an immune effector cell is an intraglomerular mesangial cell. In some embodiments, an immune effector cell is an osteoclast. In some embodiments, an immune effector cell is a Langerhans cell. In some embodiments, an immune effector cell is an epithelioid cell. In some embodiments, an immune effector cell is a red pulp macrophage. In some embodiments, an immune effector cell is a peritoneal macrophage. In some embodiments, an immune effector cell is a lysomac. In some embodiments, an immune effector cell is a perivascular macrophage.

In some embodiments, an immune effector cell is a dendritic cell.

In some embodiments, an immune effector cell is a human immune cell or a humanized immune cell. In some embodiments, an immune effector cell is a human immune cell. In some embodiments, an immune effector cell is a humanized immune cell.

### Exogenous Fusogens

In some embodiments, an exogenous fusogen is about 50 to about 1,000 amino acids in length, for example, about: 50-950, 50-900, 50-850, 50-800, 50-750, 50-700, 50-650, 50-600, 50-550, 50-500, 50-450, 50-400, 50-350, 50-300, 50-250, 50-200, 75-950, 75-900, 75-850, 75-800, 75-750, 75-700, 75-650, 75-600, 75-550, 75-500, 75-450, 75-400, 75-350, 75-300, 75-250, 75-200, 100-950, 100-900, 100-850, 100-800, 100-750, 100-700, 100-650, 100-600, 100-550, 100-500, 100-450, 100-400, 100-350, 100-300, 100-250, 100-200, 125-950, 125-900, 125-850, 125-800, 125-750, 125-700, 125-650, 125-600, 125-550, 125-500, 125-450, 125-400, 125-350, 125-300, 125-250, 125-200, 150-950, 150-900, 150-850, 150-800, 150-750, 150-700, 150-650, 150-600, 150-550, 150-500, 150-450, 150-400, 150-350, 150-300, 150-250, or 150-200 amino acids in length. In some embodiments, an exogenous fusogen is about 50 to about 250 amino acids in length.

In some embodiments, an exogenous fusogen is not a mammalian fusogen (a non-mammalian fusogen). In some embodiments, an exogenous fusogen is a wild-type viral fusogen, a variant thereof, a fragment of a wild-type viral fusogen, or a fragment of a viral fusogen variant. In some embodiments, an exogenous fusogen is a wild-type viral fusogen. In some embodiments, an exogenous fusogen is a fragment of a wild-type viral fusogen. In some embodiments, an exogenous fusogen is a variant of a wild-type viral fusogen (a viral fusogen variant). In some embodiments, an exogenous fusogen is a fragment of a viral fusogen variant. In some embodiments, an exogenous fusogen is a wild-type fusion-associated small transmembrane (FAST) protein, a variant thereof, a fragment of a wild-type FAST protein, or a fragment of a FAST protein variant. In some embodiments, an exogenous fusogen is a wild-type FAST protein. In some embodiments, an exogenous fusogen is a fragment of a wild-type FAST protein. In some embodiments, an exogenous fusogen is a variant of a wild-type FAST protein (a FAST protein variant). In some embodiments, an exogenous fusogen is a fragment of a FAST protein variant.

In some embodiments, an exogenous fusogen is derived from an avian orthorcovirus (ARV), a Nelson Bay virus (NB), a Broome orthoreovirus (BroV), a reptilian orthoreovirus (RRV), a Baboon orthorcovirus (BRV), a group C aquareovirus (AqRV-C), a group A aquareovirus (AqRV-A), a halibut reovirus, a Mahlapitisi orthorcovirus (MaRV), a Muscovy duck orthorcovirus (MdRV), a reptilian Testudine orthoreovirus (RRVtes), a Piscine orthorcovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaccous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), or a human rotavirus species B NSP-1 (HuRVB), or a variant thereof.

In some embodiments, a variant of a specified viral fusogen is substantially similar to said viral fusogen, for example, has at least about: 80%, 90%, 95%, 96%, 97%, 98%, or 99% amino acid sequence identity to the viral fusogen. In some embodiments, a variant of a specified viral fusogen has one or more functional properties of said viral fusogen.

In some embodiments, an exogenous fusogen comprises an amino acid sequence having at least about 50% sequence identity to at least one sequence set forth in SEQ ID NOs: 1-20, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:1-20. In some embodiments, an exogenous fusogen comprises an amino acid sequence having at least about 80% sequence identity to at least one sequence set forth in SEQ ID NOs: 1-20. In some embodiments, an exogenous fusogen comprises an amino acid sequence having at least about 90% sequence identity to at least one sequence set forth in SEQ ID NOs: 1-20. In some embodiments, an exogenous fusogen comprises an amino acid sequence having at least about 95% sequence identity to at least one sequence set forth in SEQ ID NOs: 1-20. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to at least one sequence set forth in SEQ ID NOs:1-20.

In some embodiments, an exogenous fusogen comprises at least one amino acid addition, deletion, and/or substitution (e.g., at least one conservative substitution, such as highly conservative amino acid substitution), relative to a wild-type fusogen sequence (e.g., a sequence set forth in any one of SEQ ID NOs: 1-20). For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen is derived from an avian orthoreovirus (ARV), a Nelson Bay virus (NB), a Broome orthoreovirus (BroV), a reptilian orthoreovirus (RRV), a Baboon orthoreovirus (BRV), a group C aquareovirus (AqRV-C), a group A aquareovirus (AqRV-A), a halibut reovirus, a Mahlapitisi orthoreovirus (MaRV), a Muscovy duck orthoreovirus (MdRV), a reptilian Testudine orthoreovirus (RRVtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaceous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), or a human rotavirus species B NSP-1 (HuRVB).

In some embodiments, an exogenous fusogen comprises an amino acid sequence having 100% sequence identity to a sequence set forth in SEQ ID NOs:1-20.

In some embodiments, an exogenous fusogen is derived from an avian orthoreovirus (ARV), a Nelson Bay virus (NB), a Broome orthoreovirus (BroV), a reptilian orthorcovirus (RRV), a Baboon orthorcovirus (BRV), a group C aquarcovirus (AqRV-C), a group A aquarcovirus (AqRV-A), a halibut reovirus, a Mahlapitisi orthoreovirus (MaRV), a Muscovy duck orthoreovirus (MdRV), a reptilian Testudine orthoreovirus (RRVtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaccous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), a human rotavirus species B NSP-1 (HuRVB), or a veiled chameleon reovirus (VC), or a variant thereof.

In some embodiments, an exogenous fusogen comprises an amino acid sequence having at least about 50% sequence identity to at least one sequence set forth in SEQ ID NOs: 1-20 and 152, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:1-20 and 152. In some embodiments, an exogenous fusogen comprises an amino acid sequence having at least about 80% sequence identity to at least one sequence set forth in SEQ ID NOs: 1-20 and 152. In some embodiments, an exogenous fusogen comprises an amino acid sequence having at least about 90% sequence identity to at least one sequence set forth in SEQ ID NOs: 1-20 and 152. In some embodiments, an exogenous fusogen comprises an amino acid sequence having at least about 95% sequence identity to at least one sequence set forth in SEQ ID NOs: 1-20 and 152. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to at least one sequence set forth in SEQ ID NOs: 1-20 and 152.

In some embodiments, an exogenous fusogen comprises at least one amino acid addition, deletion, and/or substitution (e.g., at least one conservative substitution, such as highly conservative amino acid substitution), relative to a wild-type fusogen sequence (e.g., a sequence set forth in any one of SEQ ID NOs: 1-20 and 152). For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen is derived from an avian orthoreovirus (ARV), a Nelson Bay virus (NB), a Broome orthoreovirus (BroV), a reptilian orthoreovirus (RRV), a Baboon orthoreovirus (BRV), a group C aquarcovirus (AqRV-C), a group A aquareovirus (AqRV-A), a halibut reovirus, a Mahlapitisi orthoreovirus (MaRV), a Muscovy duck orthoreovirus (MdRV), a reptilian Testudine orthoreovirus (RRVtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaceous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), a human rotavirus species B NSP-1 (HuRVB), or a veiled chameleon reovirus (VC).

In some embodiments, an exogenous fusogen comprises an amino acid sequence having 100% sequence identity to a sequence set forth in SEQ ID NOs: 1-20 and 152.

In some embodiments, an exogenous fusogen is an engineered fusogen (e.g., a recombinantly engineered fusogen).

In some embodiments, an exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof, or any combination of the foregoing.

In some embodiments, an exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
or any combination of the foregoing.

In some embodiments, an exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
or any combination of the foregoing.

In some embodiments, an exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC;
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC;
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC;
or any combination of the foregoing.

In some embodiments, an exogenous fusogen comprises:
a) an extracellular domain of a viral fusogen derived from a BRV, a BroV, an ARV, an AqRV-A, a RRV, or a variant thereof,
b) a transmembrane domain of a viral fusogen derived from a PsRV, a MdRV, an ARV, a RRV, or a variant thereof,
c) an intracellular domain of a viral fusogen derived from a RRV, an AqRV-C, a MdRV, a Nelson Bay virus, a MaRV, an ARVch, or a variant thereof, or any combination of the foregoing.

In some embodiments, an exogenous fusogen comprises:
a) an extracellular domain of a viral fusogen derived from a BRV, a BroV, an ARV, an AqRV-A, or a RRV,
b) a transmembrane domain of a viral fusogen derived from a PsRV, a MdRV, an ARV, or a RRV,
c) an intracellular domain of a viral fusogen derived from a RRV, an AqRV-C, a MdRV, a Nelson Bay virus, a MaRV, or an ARVch,
or any combination of the foregoing.

In some embodiments, an exogenous fusogen comprises an amino acid sequence having at least about 50% sequence identity to at least one sequence set forth in SEQ ID NOs:21-53, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:21-53. In some embodiments, an exogenous fusogen comprises an amino acid sequence having at least about 80% sequence identity to at least one sequence set forth in SEQ ID NOs:21-53. In some embodiments, an exogenous fusogen comprises an amino acid sequence having at least about 90% sequence identity to at least one sequence set forth in SEQ ID NOs:21-53. In some embodiments, an exogenous fusogen comprises an amino acid sequence having at least about 95% sequence identity to at least one sequence set forth in SEQ ID NOs:21-53. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to at least one sequence set forth in SEQ ID NOs:21-53.

In some embodiments, an exogenous fusogen comprises at least one amino acid addition, deletion, and/or substitution (e.g., at least one conservative substitution such as highly conservative amino acid substitution), relative to an exogenous fusogen sequence set forth in any one of SEQ ID NOs:21-53. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen comprises an amino acid sequence having 100% sequence identity to a sequence set forth in SEQ ID NOs:21-53.

In some embodiments, an exogenous fusogen comprises:
a) an extracellular domain of a viral fusogen derived from a BRV, a BroV, an ARV, an AqRV-A, or a RRV,
b) a transmembrane domain of a viral fusogen derived from a PsRV, a MdRV, a RRV, or an ARV,
c) an intracellular domain of a viral fusogen derived from a RRV, an AqRV-C, a MdRV, a Nelson Bay virus, a MaRV, or an ARVch,
or any combination of the foregoing.

In some embodiments, an exogenous fusogen comprises an amino acid sequence having at least about 50% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, and 110, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, and 110. In some embodiments, an exogenous fusogen comprises an amino acid sequence having at least about 80% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, and 110. In some embodiments, an exogenous fusogen comprises an amino acid sequence having at least about 90% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, and 110. In some embodiments, an exogenous fusogen comprises an amino acid sequence having at least about 95% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, and 110. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, and 110.

In some embodiments, an exogenous fusogen comprises at least one amino acid addition, deletion, and/or substitution (e.g., at least one conservative substitution such as highly conservative amino acid substitution), relative to an exogenous fusogen sequence set forth in any one of SEQ ID NOs:67, 151, 70, 72, 74, 76, 78, 80, 82, 84, 86, 88, 90, 92, 94, 96, 98, 100, 102, 104, 106, 108, and 110. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen comprises an amino acid sequence having 100% sequence identity to a sequence set forth in SEQ ID NOs:67, 151, 70, 72, 74, 76, 78, 80, 82, 84, 86, 88, 90, 92, 94, 96, 98, 100, 102, 104, 106, 108, and 110.

In some embodiments, an exogenous fusogen comprises an amino acid sequence having at least about 50% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137. In some embodiments, an exogenous fusogen comprises an amino acid sequence having at least about 80% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137. In some embodiments, an exogenous fusogen comprises an amino acid sequence having at least about 90% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137. In some embodiments, an exogenous fusogen comprises an amino acid sequence having at least about 95% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to at least one sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137.

In some embodiments, an exogenous fusogen comprises at least one amino acid addition, deletion, and/or substitution (e.g., at least one conservative substitution such as highly conservative amino acid substitution), relative to an exogenous fusogen sequence set forth in any one of SEQ ID NOs:67, 151, 70, 72, 74, 76, 78, 80, 82, 84, 86, 88, 90, 92, 94, 96, 98, 100, 102, 104, 106, 108, 110, and 112-137. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen comprises an amino acid sequence having 100% sequence identity to a sequence set forth in SEQ ID NOs:67, 151, 70, 72, 74, 76, 78, 80, 82, 84, 86, 88, 90, 92, 94, 96, 98, 100, 102, 104, 106, 108, 110, and 112-137.

In some embodiments, an exogenous fusogen comprises an amino acid sequence having at least about 50% sequence identity to at least one sequence set forth in SEQ ID NOs:112-137, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:112-137. In some embodiments, an exogenous fusogen comprises an amino acid sequence having at least about 80% sequence identity to at least one sequence set forth in SEQ ID NOs:112-137. In some embodiments, an exogenous fusogen comprises an amino acid sequence having at least about 90% sequence identity to at least one sequence set forth in SEQ ID NOs:112-137. In some embodiments, an exogenous fusogen comprises an amino acid sequence having at least about 95% sequence identity to at least one sequence set forth in SEQ ID NOs:112-137. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to at least one sequence set forth in SEQ ID NOs: 112-137.

In some embodiments, an exogenous fusogen comprises at least one amino acid addition, deletion, and/or substitution (e.g., at least one conservative substitution such as highly conservative amino acid substitution) relative to an exogenous fusogen sequence set forth in any one of SEQ ID NOs: 112-137. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen comprises an amino acid sequence having 100% sequence identity to a sequence set forth in SEQ ID NOs: 112-137.

In some embodiments, an exogenous fusogen comprises an amino acid sequence having at least about 50% sequence identity to at least one sequence set forth in SEQ ID NOs:113-116, 120, and 126, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs: 113-116, 120, and 126. In some embodiments, an exogenous fusogen comprises an amino acid sequence having at least about 80% sequence identity to at least one sequence set forth in SEQ ID NOs: 113-116, 120, and 126. In some embodiments, an exogenous fusogen comprises an amino acid sequence having at least about 90% sequence identity to at least one sequence set forth in SEQ ID NOs: 113-116, 120, and 126. In some embodiments, an exogenous fusogen comprises an amino acid sequence having at least about 95% sequence identity to at least one sequence set forth in SEQ ID NOs: 113-116, 120, and 126. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to at least one sequence set forth in SEQ ID NOs:113-116, 120, and 126.

In some embodiments, an exogenous fusogen comprises at least one amino acid addition, deletion, and/or substitution (e.g., at least one conservative substitution such as highly conservative amino acid substitution) relative to an exogenous fusogen sequence set forth in any one of SEQ ID NOs: 113-116, 120, and 126. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen comprises an amino acid sequence having 100% sequence identity to a sequence set forth in SEQ ID NOs: 113-116, 120, and 126.

In some embodiments, an exogenous fusogen comprises an amino acid sequence having at least about 50% sequence identity to at least one sequence set forth in SEQ ID NOs: 138-149, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:138-149. In some embodiments, an exogenous fusogen comprises an amino acid sequence having at least about 80% sequence identity to at least one sequence set forth in SEQ ID NOs:138-149. In some embodiments, an exogenous fusogen comprises an amino acid sequence having at least about 90% sequence identity to at least one sequence set forth in SEQ ID NOs:138-149. In some embodiments, an exogenous fusogen comprises an amino acid sequence having at least about 95% sequence identity to at least one sequence set forth in SEQ ID NOs:138-149. In some embodiments, an exogenous fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to at least one sequence set forth in SEQ ID NOs: 138-149.

In some embodiments, an exogenous fusogen comprises at least one amino acid addition, deletion, and/or substitution (e.g., at least one conservative substitution such as highly conservative amino acid substitution) relative to an exogenous fusogen sequence set forth in any one of SEQ ID NOs: 138-149. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21, or 22; or about: 1-22, 1-20, 2-20, 2-18, 3-18, 3-16, 4-16, 4-14, 5-14, 5-12, 6-12, 6-10, 7-10, or 7-8.

In some embodiments, an exogenous fusogen comprises an amino acid sequence having 100% sequence identity to a sequence set forth in SEQ ID NOs: 138-149.

In some embodiments, a polynucleotide encoding an exogenous fusogen is introduced and expressed in an immune effector cell using routine methods and readily available reagents. In some embodiments, a polynucleotide encoding an exogenous fusogen is synthesized by conventional techniques, including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992).

In some embodiments, a polynucleotide encoding an exogenous fusogen is introduced into a cell (e.g., an immune effector cell or a mammalian cell) using a physical or chemical method, for example, by transfection, transformation, or transduction. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); diethylaminocthyl (DEAE)-dextran; electroporation; cationic liposome-mediated transfection; tungsten particle-facilitated microparticle bombardment (Johnston, Nature, 346: 776-77 (1990)); and strontium phosphate DNA co-precipitation (Brash et al., Mol. Cell Biol., 7: 2031-34 (1987)). Phage or viral vectors can be introduced into cells, after growth of infectious particles in suitable packaging cells, many of which are commercially available.

In some embodiments, a retrovirus is used to deliver a polynucleotide encoding an exogenous fusogen into a cell (e.g., an immune effector cell or a mammalian cell). Retroviruses are a common tool for gene delivery (Miller, 2000, Nature 357: 455-60). Non-limiting examples of retroviruses suitable for use in particular embodiments include Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and lentivirus. Non-limiting examples of lentiviruses include human immunodeficiency virus (e.g., HIV type 1 and HIV type 2), visna-macdi virus (VMV), caprine arthritis-encephalitis virus (CAEV), equine infectious anemia virus (EIAV), feline immunodeficiency virus (FIV), bovine immune deficiency virus (BIV), and simian immunodeficiency virus (SIV).

In some embodiments, a polynucleotide encoding an exogenous fusogen is delivered into a cell (e.g., an immune effector cell or a mammalian cell) using a vector system (e.g., a lentiviral vector (LV), a retroviral vector (RV), or a transposon vector).

In some embodiments, a polynucleotide encoding an exogenous fusogen is integrated into the genome of a cell (e.g., an immune effector cell or a mammalian cell). In some embodiments, a polynucleotide encoding an exogenous fusogen is inserted into a gene. In some embodiments, a polynucleotide encoding an exogenous fusogen is extrachromosomal in a cell (e.g., an immune effector cell or a mammalian cell).

### Expression of Exogenous Fusogens

In some embodiments, an immune effector cell expresses an exogenous fusogen (e.g., an engineered fusogen) disclosed herein. In some embodiments, a mammalian cell expresses an exogenous fusogen (e.g., an engineered fusogen) disclosed herein.

In some embodiments, the level of an exogenous fusogen in an immune effector cell or a mammalian cell is conditionally (e.g., tunable, reversible, spatial control, and/or temporal control) regulatable (e.g., inducible or suppressible), for example, at the level of transcription, protein synthesis, protein degradation, or a combination thereof. In some embodiments, the level of an exogenous fusogen in an immune effector cell or a mammalian cell is conditionally inducible. In some embodiments, the level of an exogenous fusogen in an immune effector cell or a mammalian cell is conditionally suppressible. In some embodiments, the level of an exogenous fusogen in an immune effector cell or a mammalian cell is conditionally regulated by controlling gene expression (e.g., reversibly or irreversibly).

In some embodiments, the level (e.g., transcription level) of an exogenous fusogen in an immune effector cell or a mammalian cell is conditionally regulatable (e.g., inducible or suppressible) by a small molecule, a light-switchable system, a ligand-switchable system, or a combination thereof.

In some embodiments, the level (e.g., transcription level) of an exogenous fusogen in an immune effector cell or a mammalian cell is conditionally regulatable (e.g., inducible or suppressible) by a small molecule. Non-limiting examples of small molecules include tetracycline (TET), doxycycline (DOX), caffeine, 4-hydroxytamoxifen, estrogen, ecdysone, abscisic acid, mifepristone, xylose, FKBP12-rapamycin, and HCV NS3/4A protease inhibitors. In some embodiments, a small molecule comprises TET or DOX. In some embodiments, a small molecule comprises TET. In some embodiments, a small molecule comprises DOX. In some embodiments, a small molecule comprises caffeine. In some embodiments, a small molecule comprises 4-hydroxytamoxifen. In some embodiments, a small molecule comprises estrogen. In some embodiments, a small molecule comprises ecdysone. In some embodiments, a small molecule comprises abscisic acid. In some embodiments, a small molecule comprises mifepristone. In some embodiments, a small molecule comprises xylose. In some embodiments, a small molecule comprises KBP12-rapamycin. In some embodiments, a small molecule comprises an HCV NS3/4A protease inhibitor. In some embodiments, an HCV NS3/4A protease inhibitor comprises grazoprevir.

In some embodiments, an immune effector cell comprises a transcriptional control element responsive to a small molecule described herein operably linked to a nucleic acid encoding an exogenous fusogen.

In some embodiments, the level (e.g., transcription level) of an exogenous fusogen in an immune effector cell or a mammalian cell is conditionally regulatable (e.g., inducible or suppressible) by a light-switchable system. In some embodiments, a light-switchable system responds to light in the range of about 450-700 nm, for example, in the range of about 450-500 nm (e.g., about 450 nm) or about 620-700 nm (e.g., about 660 nm).

In some embodiments, a light-switchable system (e.g., a light-inducible system) comprises one or more customizable zinc finger transcription factors. In some embodiments, a light-switchable system comprises GIGANTEA and the light oxygen voltage (LOV) domain of Flavin-Binding Kelch Repeat F-Box 1 (FKF1). See, e.g., Polstein & Gersbach, Light-inducible spatiotemporal control of gene activation by customizable zinc finger transcription factors, J Am Chem Soc. 134(40):16480-3 (2012), the contents of which are incorporated by reference herein in their entirety. In some embodiments, an immune effector cell comprises a transcriptional control element responsive to a customizable zinc finger transcription factor operably linked to a nucleic acid encoding an exogenous fusogen. Non-limiting examples of a transcriptional control element include three repeats of a binding site for GI-ZFP1, three to nine repeats (e.g., 3, 6, 7, or 9 repeats) of a binding site for GI-ZFP2, and three repeats of a binding site for GI-ZFP3.

In some embodiments, a light-switchable system comprises Phytochrome B (PhyB) and phytochrome interaction factor 6 (PIF6). See, e.g., Müller et al., Control of gene expression using a red-and far-red light-responsive bi-stable toggle switch, Nat Protoc. 9(3):622-32 (2014), the contents of which are incorporated by reference herein in their entirety. In some embodiments, an immune effector cell comprises: (a) a tetracycline operator (tetO) motif operably linked to a nucleic acid encoding an exogenous fusogen, (b) a phytochrome-interacting factor 6 (PIF6) sequence (e.g., amino acids 1-100 of PIF6 from Arabidopsis thaliana) fused to tetracycline repressor (TetR), and (c) A. thaliana red- and far-red light receptor phytochrome B (PhyB) (e.g., amino acids 1-650 of PhyB) linked to VP16 transactivation domain and a nuclear localization sequence (NLS).

In some embodiments, the level (e.g., transcription level) of an exogenous fusogen in an immune effector cell or a mammalian cell is conditionally regulatable (e.g., inducible or suppressible) by a ligand-switchable system. In some embodiments, a ligand-switchable system comprises a synthetic Notch receptor (synNotch), a modular extracellular sensor, a synthetic intramembrane proteolysis receptor (SNIPR), or a combination thereof. In some embodiments, a ligand-switchable system comprises a synNotch, a modular extracellular sensor, or a SNIPR. In some embodiments, a ligand-switchable system comprises a synNotch. In some embodiments, a ligand-switchable system comprises a modular extracellular sensor. In some embodiments, a ligand-switchable system comprises SNIPR. In some embodiments, an immune effector cell comprises: (a) a nucleic acid encoding a binding-triggered transcriptional switch (e.g., synNotch, a modular extracellular sensor, or SNIPR), and (b) a transcriptional control element responsive to the binding-triggered transcriptional switch operably linked to a nucleic acid encoding an exogenous fusogen. See, e.g., published PCT applications WO2016138034, WO2013022739, and WO2021061791, the contents of which are incorporated by reference herein in their entirety.

### Targeting Molecules

In some embodiments, an immune effector cell further comprises a nucleotide sequence encoding a cell-targeting molecule.

In some embodiments, a cell-targeting molecule recognizes a cell-surface marker for one or more cancer cells, neurons, oligodendrocytes, microglial cells, neural stem cells, hematopoietic stem cells, astrocytes, or pancreatic beta cells.

In some embodiments, a cell-targeting molecule recognizes a cell-surface marker for one or more cancer cells. In some embodiments, a cell surface marker is a tumor-associated antigen “TAA.” In some embodiments, a tumor-associated antigen is expressed at a higher level in tumor cells than in non-tumor cells. In some embodiments, a tumor-associated antigen is a universal tumor antigen, i.e., broadly expressed by most types of tumors. In some embodiments, a tumor-associated antigen is exclusively expressed in a specific type of tumor cells. In some embodiments, a tumor-associated antigen is expressed at low levels in non-tumor cells. In some embodiments, a tumor-associated antigen is not expressed in non-tumor cells.

In some embodiments, a tumor-associated antigen is a “tumor-specific antigen” or “TSA,” i.e., is unique to a tumor cell. In some embodiments, a TSA is a neoantigen, i.e., a protein that is unique to an individual tumor, tumor type, or class of tumors.

In some embodiments, a tumor-associated antigen is a differentiation antigen, a mutational antigen, an overexpressed cellular antigen, a viral antigen, or a combination thereof. In some embodiments, a tumor-associated antigen is a differentiation antigen. In some embodiments, a tumor-associated antigen is a mutational antigen. In some embodiments, a tumor-associated antigen is an overexpressed cellular antigen. In some embodiments, a tumor-associated antigen is a viral antigen.

In some embodiments, a cell-surface marker is expressed on a solid tumor cell, a hematologic cancer cell, or both. In some embodiments, a cell-surface marker is expressed on a hematologic cancer cell. In some embodiments, a cell-surface marker is expressed on a solid tumor cell and a hematologic cancer cell.

In some embodiments, a cell-surface marker comprises a B-cell lymphoma-derived antigen. Non-limiting examples of B-cell lymphoma-derived antigens include B-cell activating factor (BAFF) receptor, B-cell maturation antigen (BCMA), CD3, CD5, CD19, CD20, CD22, CD28, CD30, CD38, CD79B, and programmed cell death protein 1 (PD-1).

In some embodiments, a cell-surface marker comprises a lung cancer-derived antigen. Non-limiting examples of lung cancer-derived antigens include AXL, B7 homolog 3 protein (B7-H3), carcinoembryonic antigen (CEA), CD70, claudin 18.2 (CLDN18.2), delta-like ligand 3 (DLL3), disialoganglioside (GD2), epidermal growth factor receptor (EGFR), glypican-3 (GPC3), guanylyl cyclase C (GUCY2C), human epidermal growth factor receptor 2 (HER2), Kita-Kyushu lung cancer antigen-1 (KK-LC-1), Lewis Y (LEY), mesothelin (MSLN), MUCIN 1 (MUC1), NEW YORK esophageal squamous cell carcinoma 1 (NY-ESO-1), positive programmed death-ligand 1 (PD-L1), prostate specific membrane antigen (PSMA), prostate stem cell antigen (PSCA), receptor-tyrosine-kinase like orphan receptor 1 (ROR1), transforming growth factor beta (TGF-β), Kirsten rat sarcoma virus (KRAS) G12D, melanoma antigen recognized by T cells 1 (MART-1), melanoma-associated antigen 3 (MAGE-A3), tumor protein p53 (TP53), FMS-like tyrosine kinase 3 (FLT3), and alkaline phosphatase placental-like 2 (ALPPL2).

In some embodiments, a cell-targeting molecule recognizes a cell-surface marker for one or more neurons. Non-limiting examples of cell-surface markers for neurons include L1 cell adhesion molecule (LICAM), neurexin 3, vesicular glutamate transporter 1 (VGLUT1), vesicular inhibitory amino acid transporter (VIAAT), neuroligin 1, neuroligin 2, neural cell adhesion molecule 1 (NCAM1), vesicular acetylcholine transporter (VAChT), folate receptor-1 (FOLR1), gamma-aminobutyric acid B receptor 1 (GABA(b)R1), GABA(b)R2, glutamate ionotropic receptor NMDA type subunit 1 (GRIN1), GRIN2B, and solute carrier family 6 member 4 (SLC6A4).

In some embodiments, a cell-targeting molecule recognizes a cell-surface marker for one or more oligodendrocytes. Non-limiting examples of cell-surface markers for oligodendrocytes include neural/glial antigen 2 (NG2), oligodendrocyte marker 01, oligodendrocyte marker 04, A2B5, and myelin-oligodendrocyte glycoprotein (MOG).

In some embodiments, a cell-targeting molecule recognizes a cell-surface marker for one or more microglial cells. Non-limiting examples of cell-surface markers for microglial cells include P2Y12, macrophage colony-stimulating factor receptor (M-CSFR), and CX3C motif chemokine receptor 1 (CX3CR1).

In some embodiments, a cell-targeting molecule recognizes a cell-surface marker for one or more neural stem cells. Non-limiting examples of cell-surface markers for neural stem cells include CD133 and CD49F.

In some embodiments, a cell-targeting molecule recognizes a cell-surface marker for one or more hematopoietic stem cells. Non-limiting examples of cell-surface markers for hematopoietic stem cells include stem cell antigen-1 (Sca-1), CD27, CD34, CD38, CD43, CD117, and CD150.

In some embodiments, a cell-targeting molecule recognizes a cell-surface marker for one or more astrocytes. Non-limiting examples of cell-surface markers for astrocytes include A2B5, connexin 43, and aquaporin-4 (AQP-4).

In some embodiments, a cell-targeting molecule recognizes a cell-surface marker for one or more pancreatic beta cells. Non-limiting examples of cell-surface markers for pancreatic beta cells include CD71 and CD24.

In some embodiments, a cell-targeting molecule recognizes:
a) LICAM, neurexin 3, VGLUT1, VIAAT, neuroligin 1, neuroligin 2, NCAM1, VACHT, FOLR1, GABA(b)R1, GABA(b)R2, GRIN1, GRIN2B, or SLC6A4,
b) NG2, oligodendrocyte marker 01, oligodendrocyte marker 04, A2B5, or MOG,
c) P2Y12, M-CSFR, or CX3CR1,
d) CD133 or CD49F,
e) Sca-1, CD27, CD34, CD38, CD43, CD117, or CD150,
f) A2B5, connexin 43, or AQP-4,
g) CD71 or CD24,
h) BAFF receptor, BCMA, CD3, CD5, CD19, CD20, CD22, CD28, CD30, CD38, CD79B, or PD-1, or
i) AXL, B7-H3, CEA, CD70, CLDN18.2, DLL3, GD2, EGFR, GPC3, GUCY2C, HER2, KK-LC-1, LEY, MSLN, MUC1, NY-ESO-1, PD-L1, PSMA, PSCA, ROR1, TGF-β, KRAS G12D, MART-1, MAGE-A3, TP53, FLT3, or ALPPL2.

In some embodiments, a cell-targeting molecule recognizes BAFF receptor, BCMA, CD3, CD5, CD19, CD20, CD22, CD28, CD30, CD38, CD79B, or PD-1.

In some embodiments, a cell-targeting molecule recognizes AXL, B7-H3, CEA, CD70, CLDN18.2, DLL3, GD2, EGFR, GPC3, GUCY2C, HER2, KK-LC-1, LEY, MSLN, MUC1, NY-ESO-1, PD-L1, PSMA, PSCA, ROR1, TGF-β, KRAS G12D, MART-1, MAGE-A3, TP53, FLT3, or ALPPL2.

In some embodiments, a cell-targeting molecule comprises a native protein.

In some embodiments, a cell-targeting molecule comprises an antibody or an antigen-binding fragment thereof, a T cell receptor (TCR), an integrin, a cell adhesion molecule, or a combination thereof.

In some embodiments, a cell-targeting molecule comprises an antibody or an antigen-binding fragment thereof. In some embodiments, an antigen-binding fragment comprises a single-chain fragment variable (scFv), a variable heavy domain of heavy chain (V_{HH}), a fragment antigen-binding (Fab), a Fab′ or a F(ab′)2.

In some embodiments, a cell-targeting molecule comprises a TCR. In some embodiments, a cell-targeting molecule comprises a patient-derived TCR.

In some embodiments, a cell-targeting molecule comprises an integrin. In some embodiments, an integrin comprises integrin beta-1 (ITGβ1), integrin alpha-5 (ITGα5), lymphocyte function-associated antigen 1 (LFA-1), integrin subunit alpha V (ITGαV), or a combination thereof.

In some embodiments, a cell-targeting molecule comprises a cell-adhesion molecule. In some embodiments, a cell-adhesion molecule comprises intercellular adhesion molecule-1 (ICAM-1), vascular cell adhesion protein-1 (VCAM-1), neural cell adhesion molecule 1 (N-CAM1), platelet endothelial cell adhesion molecule-1 (PE-CAM1), or a combination thereof.

In some embodiments, a cell-targeting molecule comprises an engineered protein. In some embodiments, an engineered protein comprises an antibody or an antigen-binding fragment thereof, an engineered receptor, or a combination thereof. In some embodiments, an engineered receptor comprises a chimeric antigen receptor (CAR), an engineered T cell receptor (TCR), a synthetic Notch receptor (synNotch), a modular extracellular sensor, a synthetic intramembrane proteolysis receptor (SNIPR), a cell adhesion molecule, or a combination thereof. In some embodiments, a cell-targeting molecule comprises an anti-CD19 single chain variable fragment (scFv).

In some embodiments, a cell-targeting molecule further comprises an intracellular adhesion domain. In some embodiments, an intracellular adhesion domain comprises an intracellular domain of vascular cell adhesion molecule-1 (VCAM-1), P-Selectin, L-Selectin, MHC class I-restricted T cell-associated molecule (CRTAM), integrin beta2 (ITGβ2), integrin beta1 (ITGβ1), E-Selectin, P-Cadherin, CD103, E-Cadherin, N-Cadherin, mucin-4 (MUC-4), intercellular adhesion molecule 1 (ICAM1), or a combination thereof.

In some embodiments, a cell-targeting molecule recognizes at least two cell-surface markers.

### Expression of Cell-Targeting Molecules

In some embodiments, an immune effector cell expresses a cell-targeting molecule. In some embodiments, a mammalian cell expresses a cell-targeting molecule.

In some embodiments, the level of a cell-targeting molecule in an immune effector cell or a mammalian cell is conditionally (e.g., tunable, reversible, spatial control, and/or temporal control) regulatable (e.g., inducible or suppressible), for example, at the level of transcription, protein synthesis, protein degradation, or a combination thereof.

In some embodiments, the level of a cell-targeting molecule in an immune effector cell or a mammalian cell is conditionally inducible. In some embodiments, the level of a cell-targeting molecule in an immune effector cell or a mammalian cell is conditionally suppressible. In some embodiments, the level of a cell-targeting molecule in an immune effector cell or a mammalian cell is conditionally regulated by controlling gene expression (e.g., reversibly or irreversibly).

In some embodiments, the level (e.g., transcription level) of a cell-targeting molecule in an immune effector cell or a mammalian cell is conditionally regulatable (e.g., inducible or suppressible) by a small molecule, a light-switchable system, a ligand-switchable system, or a combination thereof.

In some embodiments, the level (e.g., transcription level) of a cell-targeting molecule in an immune effector cell or a mammalian cell is conditionally regulatable (e.g., inducible or suppressible) by a small molecule. Non-limiting examples of small molecules include tetracycline (TET), doxycycline (DOX), caffeine, 4-hydroxytamoxifen, estrogen, ccdysone, abscisic acid, mifepristone, xylose, FKBP12-rapamycin, and HCV NS3/4A protease inhibitors. In some embodiments, a small molecule comprises TET or DOX. In some embodiments, a small molecule comprises TET. In some embodiments, a small molecule comprises DOX. In some embodiments, a small molecule comprises caffeine. In some embodiments, a small molecule comprises 4-hydroxytamoxifen. In some embodiments, a small molecule comprises estrogen. In some embodiments, a small molecule comprises ecdysone. In some embodiments, a small molecule comprises abscisic acid. In some embodiments, a small molecule comprises mifepristone. In some embodiments, a small molecule comprises xylose. In some embodiments, a small molecule comprises KBP12-rapamycin. In some embodiments, a small molecule comprises an HCV NS3/4A protease inhibitor. In some embodiments, an HCV NS3/4A protease inhibitor comprises grazoprevir.

In some embodiments, an immune effector cell comprises a transcriptional control element responsive to a small molecule described herein operably linked to a nucleic acid encoding a cell-targeting molecule.

In some embodiments, the level (e.g., transcription level) of a cell-targeting molecule in an immune effector cell or a mammalian cell is conditionally regulatable (e.g., inducible or suppressible) by a light-switchable system. In some embodiments, a light-switchable system responds to light in the range of about 450-700 nm, for example, in the range of about 450-500 nm (e.g., about 450 nm) or about 620-700 nm (e.g., about 660 nm).

In some embodiments, a light-switchable system (e.g., a light-inducible system) comprises one or more customizable zinc finger transcription factors. In some embodiments, a light-switchable system comprises GIGANTEA and the light oxygen voltage (LOV) domain of Flavin-Binding Kelch Repeat F-Box 1 (FKF1). See, e.g., Polstein & Gersbach, Light-inducible spatiotemporal control of gene activation by customizable zinc finger transcription factors, J Am Chem Soc. 134(40):16480-3 (2012), the contents of which are incorporated by reference herein in their entirety. In some embodiments, an immune effector cell comprises a transcriptional control element responsive to a customizable zinc finger transcription factor operably linked to a nucleic acid encoding a cell-targeting molecule. Non-limiting examples of a transcriptional control element include three repeats of a binding site for GI-ZFP1, three to nine repeats (e.g., 3, 6, 7, or 9 repeats) of a binding site for GI-ZFP2, and three repeats of a binding site for GI-ZFP3.

In some embodiments, a light-switchable system comprises Phytochrome B (PhyB) and phytochrome interaction factor 6 (PIF6). See, e.g., Müller et al., Control of gene expression using a red- and far-red light-responsive bi-stable toggle switch, Nat Protoc. 9(3):622-32 (2014), the contents of which are incorporated by reference herein in their entirety. In some embodiments, an immune effector cell comprises: (a) a tetracycline operator (tetO) motif operably linked to a nucleic acid encoding a cell-targeting molecule, (b) a phytochrome-interacting factor 6 (PIF6) sequence (e.g., amino acids 1-100 of PIF6 from Arabidopsis thaliana) fused to tetracycline repressor (TetR), and (c) A. thaliana red- and far-red light receptor phytochrome B (PhyB) (e.g., amino acids 1-650 of PhyB) linked to VP16 transactivation domain and a nuclear localization sequence (NLS).

In some embodiments, the level (e.g., transcription level) of a cell-targeting molecule in an immune effector cell or a mammalian cell is conditionally regulatable (e.g., inducible or suppressible) by a ligand-switchable system. In some embodiments, a ligand-switchable system comprises a synthetic Notch receptor (synNotch), a modular extracellular sensor, a synthetic intramembrane proteolysis receptor (SNIPR), or a combination thereof. In some embodiments, a ligand-switchable system comprises a synNotch, a modular extracellular sensor, or a SNIPR. In some embodiments, a ligand-switchable system comprises a synNotch. In some embodiments, a ligand-switchable system comprises a modular extracellular sensor. In some embodiments, a ligand-switchable system comprises SNIPR. In some embodiments, an immune effector cell comprises: (a) a nucleic acid encoding a binding-triggered transcriptional switch (e.g., synNotch, a modular extracellular sensor, or SNIPR), and (b) a transcriptional control element responsive to the binding-triggered transcriptional switch operably linked to a nucleic acid encoding a cell-targeting molecule. See, e.g., published PCT applications WO2016138034, WO2013022739, and WO2021061791, the contents of which are incorporated by reference herein in their entirety.

### Payloads

In some embodiments, an immune effector cell further comprises a nucleotide sequence encoding a payload. In some embodiments, a mammalian cell further comprises a nucleotide sequence encoding a payload.

In some embodiments, a payload comprises a therapeutic agent.

In some embodiments, a payload induces cell death. In some embodiments, a payload induces apoptosis, pyroptosis, or necroptosis.

In some embodiments, a payload comprises a truncated p15 BID (tBID), caspase 1, receptor-interacting protein kinase-3 (RIPK3), caspase-3, caspase-7, caspase-8, caspase-9, mixed lineage kinase domain-like pseudokinase (MLKL), Gasdermin-D (GSDMD), Gasdermin-E (GSDME), nerve injury-induced protein 1 (Ninj1), or a combination thereof.

In some embodiments, a payload comprises a cytokine.

In some embodiments, a payload induces an inflammatory immune response.

In some embodiments, a payload comprises stimulator of interferon genes (STING), interferon γ, high mobility group box 1 (HMGB1), interleukin-2 (IL-2), IL-12, IL-18, tumor necrosis factor alpha (TNFα), or a combination thereof.

In some embodiments, a payload comprises a checkpoint inhibitor. In some embodiments, a payload comprises an antibody that binds to comprises an antibody that binds to PD-1 (Programmed cell death protein-1), PD-L1, CTLA-4, LAG3, or a combination thereof.

In some embodiments, a payload is human or humanized. In some embodiments, a payload is human. In some embodiments, a payload is humanized.

### Expression of Payloads

In some embodiments, an immune effector cell expresses a payload. In some embodiments, a mammalian cell expresses a payload. In some embodiments, a fused cell expresses a payload.

In some embodiments, the level of a payload in an immune effector cell, a mammalian cell, or a fused cell is conditionally (e.g., tunable, reversible, spatial control, and/or temporal control) regulatable (e.g., inducible or suppressible), for example, at the level of transcription, protein synthesis, protein degradation, or a combination thereof.

In some embodiments, the level of a payload in an immune effector cell, a mammalian cell, or a fused cell is conditionally inducible. In some embodiments, the level of a payload in an immune effector cell, a mammalian cell, or a fused cell is conditionally suppressible. In some embodiments, the level of a payload in an immune effector cell, a mammalian cell, or a fused cell is conditionally regulated by controlling gene expression (e.g., reversibly or irreversibly).

In some embodiments, the level (e.g., transcription level) of a payload in an immune effector cell, a mammalian cell, or a fused cell is conditionally regulatable (e.g., inducible or suppressible) by a small molecule, a light-switchable system, a ligand-switchable system, or a combination thereof.

In some embodiments, the level (e.g., transcription level) of a payload in an immune effector cell, a mammalian cell, or a fused cell is conditionally regulatable (e.g., inducible or suppressible) by a small molecule. Non-limiting examples of small molecules include tetracycline (TET), doxycycline (DOX), caffeine, 4-hydroxytamoxifen, estrogen, ecdysone, abscisic acid, mifepristone, xylose, FKBP12-rapamycin, and HCV NS3/4A protease inhibitors. In some embodiments, a small molecule comprises DOX. In some embodiments, a small molecule comprises caffeine. In some embodiments, a small molecule comprises 4-hydroxytamoxifen. In some embodiments, a small molecule comprises estrogen. In some embodiments, a small molecule comprises ecdysone. In some embodiments, a small molecule comprises abscisic acid. In some embodiments, a small molecule comprises mifepristone. In some embodiments, a small molecule comprises xylose. In some embodiments, a small molecule comprises KBP12-rapamycin. In some embodiments, a small molecule comprises an HCV NS3/4A protease inhibitor. In some embodiments, an HCV NS3/4A protease inhibitor comprises grazoprevir.

In some embodiments, an immune effector cell comprises a transcriptional control element responsive to a small molecule described herein operably linked to a nucleic acid encoding a payload.

In some embodiments, the level (e.g., transcription level) of a payload in an immune effector cell, a mammalian cell, or a fused cell is conditionally regulatable (e.g., inducible or suppressible) by a light-switchable system. In some embodiments, a light-switchable system responds to light in the range of about 450-700 nm, for example, in the range of about 450-500 nm (e.g., about 450 nm) or about 620-700 nm (e.g., about 660 nm).

In some embodiments, a light-switchable system (e.g., a light-inducible system) comprises one or more customizable zinc finger transcription factors. In some embodiments, a light-switchable system comprises GIGANTEA and the light oxygen voltage (LOV) domain of Flavin-Binding Kelch Repeat F-Box 1 (FKF1). See, e.g., Polstein & Gersbach, Light-inducible spatiotemporal control of gene activation by customizable zinc finger transcription factors, J Am Chem Soc. 134(40):16480-3 (2012), the contents of which are incorporated by reference herein in their entirety. In some embodiments, an immune effector cell comprises a transcriptional control element responsive to a customizable zinc finger transcription factor operably linked to a nucleic acid encoding a payload. Non-limiting examples of a transcriptional control element include three repeats of a binding site for GI-ZFP1, three to nine repeats (e.g., 3, 6, 7, or 9 repeats) of a binding site for GI-ZFP2, and three repeats of a binding site for GI-ZFP3.

In some embodiments, a light-switchable system comprises Phytochrome B (PhyB) and phytochrome interaction factor 6 (PIF6). See, e.g., Müller et al., Control of gene expression using a red- and far-red light-responsive bi-stable toggle switch, Nat Protoc. 9(3):622-32 (2014), the contents of which are incorporated by reference herein in their entirety. In some embodiments, an immune effector cell comprises: (a) a tetracycline operator (tetO) motif operably linked to a nucleic acid encoding a payload, (b) a phytochrome-interacting factor 6 (PIF6) sequence (e.g., amino acids 1-100 of PIF6 from Arabidopsis thaliana) fused to tetracycline repressor (TetR), and (c) A. thaliana red- and far-red light receptor phytochrome B (PhyB) (e.g., amino acids 1-650 of PhyB) linked to VP16 transactivation domain and a nuclear localization sequence (NLS).

In some embodiments, the level (e.g., transcription level) of a payload in an immune effector cell, a mammalian cell, or a fused cell is conditionally regulatable (e.g., inducible or suppressible) by a ligand-switchable system. In some embodiments, a ligand-switchable system comprises a synthetic Notch receptor (synNotch), a modular extracellular sensor, a synthetic intramembrane proteolysis receptor (SNIPR), or a combination thereof. In some embodiments, a ligand-switchable system comprises a synNotch, a modular extracellular sensor, or a SNIPR. In some embodiments, a ligand-switchable system comprises a synNotch. In some embodiments, a ligand-switchable system comprises a modular extracellular sensor. In some embodiments, a ligand-switchable system comprises SNIPR. In some embodiments, an immune effector cell comprises: (a) a nucleic acid encoding a binding-triggered transcriptional switch (e.g., synNotch, a modular extracellular sensor, or SNIPR), and (b) a transcriptional control element responsive to the binding-triggered transcriptional switch operably linked to a nucleic acid encoding a payload. See, e.g., WO2016138034, WO2013022739, and WO2021061791, the contents of which are incorporated by reference herein in their entirety.

### Mammalian Cells

Also provided herein are mammalian cells comprising a nucleotide sequence encoding any one or more exogenous fusogens disclosed herein (e.g., in the section entitled “Fusogens”) and a nucleotide sequence encoding any one or more cell-targeting molecules described herein (e.g., in the section entitled “Targeting Molecules”).

In some embodiments, a mammalian cell expresses an exogenous fusogen. In some embodiments, the level of an exogenous fusogen in an immune effector cell or a mammalian cell is conditionally (e.g., tunable, reversible, spatial control, and/or temporal control) regulatable (e.g., inducible or suppressible), for example, at the level of transcription, protein synthesis, protein degradation, or a combination thereof. In some embodiments, the level of an exogenous fusogen in an immune effector cell or a mammalian cell is conditionally inducible. In some embodiments, the level of an exogenous fusogen in an immune effector cell or a mammalian cell is conditionally suppressible. In some embodiments, the level of an exogenous fusogen in an immune effector cell or a mammalian cell is conditionally regulated by controlling gene expression (e.g., reversibly or irreversibly). In some embodiments, the level of an exogenous fusogen in an immune effector cell or a mammalian cell is conditionally regulatable (e.g., inducible or suppressible) by a small molecule, a light-switchable system, a ligand-switchable system, or a combination thereof. See, e.g., the section entitled “Expression of Exogenous Fusogens.”

In some embodiments, an immune effector cell expresses a cell-targeting molecule. In some embodiments, a mammalian cell expresses a cell-targeting molecule. In some embodiments, the level of a cell-targeting molecule in an immune effector cell or a mammalian cell is conditionally (e.g., tunable, reversible, spatial control, and/or temporal control) regulatable (e.g., inducible or suppressible), for example, at the level of transcription, protein synthesis, protein degradation, or a combination thereof. In some embodiments, the level of a cell-targeting molecule in an immune effector cell or a mammalian cell is conditionally inducible. In some embodiments, the level of a cell-targeting molecule in an immune effector cell or a mammalian cell is conditionally suppressible. In some embodiments, the level of a cell-targeting molecule in an immune effector cell or a mammalian cell is conditionally regulated by controlling gene expression (e.g., reversibly or irreversibly). In some embodiments, the level of a cell-targeting molecule in an immune effector cell or a mammalian cell is conditionally regulatable (e.g., inducible or suppressible) by a small molecule, a light-switchable system, a ligand-switchable system, or a combination thereof. See, e.g., the section entitled “Expression of Cell-Targeting Molecules.”

In some embodiments, a mammalian cell further comprising a nucleotide sequence encoding any one or more payloads is described herein (e.g., in the section entitled “Payloads”).

In some embodiments, a mammalian cell expresses a payload. In some embodiments, the level of a payload in an immune effector cell, a mammalian cell or a fused cell is conditionally (e.g., tunable, reversible, spatial control, and/or temporal control) regulatable (e.g., inducible or suppressible), for example, at the level of transcription, protein synthesis, protein degradation, or a combination thereof. In some embodiments, the level of a payload in an immune effector cell, a mammalian cell or a fused cell is conditionally inducible. In some embodiments, the level of a payload in an immune effector cell, a mammalian cell or a fused cell is conditionally suppressible. In some embodiments, the level of a payload in an immune effector cell, a mammalian cell or a fused cell is conditionally regulated by controlling gene expression (e.g., reversibly or irreversibly). In some embodiments, the level of a payload in an immune effector cell, a mammalian cell or a fused cell is conditionally regulatable (e.g., inducible or suppressible) by a small molecule, a light-switchable system, a ligand-switchable system, or a combination thereof. See, e.g., the section entitled “Expression of Payloads.”

### Compositions/Pharmaceutical Compositions/Kits

Also provided herein are compositions comprising any one or more immune effector cells disclosed herein.

Also provided herein are compositions comprising any one or more mammalian cells disclosed herein.

In some embodiments, a composition further comprises a pharmaceutically acceptable carrier. In some embodiments, a composition is a pharmaceutical composition.

In some embodiments, a composition (e.g., a pharmaceutical composition) is formulated in a serum-free (without serum or substantially free of serum) cryopreservation medium. Any suitable cryopreservation media and/or excipients may be used. In some embodiments, a cryopreservation medium comprises 5% DMSO and Dextran 40.

Also provided herein are kits comprising a container and optionally an instruction for use, wherein the container comprises any one or more compositions of pharmaceutical compositions disclosed herein.

### Methods of Treatment

Also provided herein are methods of treating cancer in a subject in need thereof, said methods comprising administering to the subject an effective dosage of immune effector cells, mammalian cells or pharmaceutical compositions disclosed herein.

Also provided herein are use of immune effector cells, mammalian cells or pharmaceutical compositions disclosed herein, for the preparation of a medicament for treating cancer in a subject in need thereof.

Also provided herein are immune effector cells, mammalian cells or pharmaceutical compositions disclosed herein for use in treating a subject having cancer.

Also provided herein are methods of killing a cancer cell, said methods comprising contacting the cancer cell with an effective dosage of immune effector cells or mammalian cells disclosed herein.

Non-limiting examples of cancers include: acute lymphoblastic leukemia (ALL); Acquired immunodeficiency syndrome (AIDS)-related cancer (e.g., Kaposi Sarcoma, AIDS-related lymphoma, primary central nervous system (CNS) lymphoma); acute myeloid leukemia (AML); adrenocortical carcinoma; anal cancer; appendix cancer; basal cell carcinoma of the skin; bile duct cancer; bladder cancer; bone cancer (including Ewing sarcoma, osteosarcoma and malignant fibrous histiocytoma); brain tumors/cancer; breast cancer; Burkitt lymphoma; carcinoid tumor (gastrointestinal); cervical cancer; childhood adrenocortical carcinoma; childhood astrocytoma; childhood atypical CNS teratoid/rhabdoid tumor; childhood bladder cancer; childhood carcinoid tumor; childhood cardiac (heart) tumors; childhood central nervous system embryonal tumors; childhood cervical cancer; childhood chordoma; childhood CNS germ cell tumors (e.g., childhood extracranial germ cell tumors, extragonadal germ cell tumors, ovarian germ cell tumors, testicular cancer); childhood colorectal cancer; childhood craniopharyngioma; childhood embryonal tumors; childhood ependymoma; childhood esophageal cancer; childhood extracranial germ cell tumor; childhood gastric (stomach) cancer; childhood gastrointestinal stromal tumors; childhood germ cell tumor; childhood intraocular melanoma; childhood laryngeal papillomatosis; childhood lung cancer; childhood melanoma; childhood mesothelioma; childhood ovarian cancer; childhood pancreatic cancer; childhood paraganglioma; childhood pheochromocytoma; childhood rhabdomyosarcoma; childhood skin cancer; childhood testicular cancer; childhood vaginal cancer; cholangiocarcinoma; chronic lymphocytic leukemia (CLL); chronic myelogenous leukemia (CML); chronic myeloproliferative neoplasms; colorectal cancer; cutaneous T-cell lymphoma (e.g. mycosis fungoides and Sèzary syndrome); Ductal carcinoma in situ (DCIS); endometrial cancer (uterine cancer); esophageal cancer; esthesioneuroblastoma; extragonadal germ cell tumor; eye (ocular) cancer; fallopian tube cancer; gallbladder cancer; gastric (stomach) cancer; gastrointestinal carcinoid tumor; gastrointestinal stromal tumors (GIST); germ cell tumors; gestational trophoblastic disease; hairy cell leukemia; head and neck cancer; hepatocellular (liver) cancer; Hodgkin lymphoma; intraocular (eye) melanoma; kidney (renal cell) cancer; Langerhans cell histiocytosis; laryngeal cancer; leukemia; liver cancer; lung cancer (non-small cell and small cell); lymphoma; male breast cancer; malignant mesothelioma; melanoma; Merkel cell carcinoma; metastatic cancer; metastatic squamous neck cancer with occult primary; midline tract carcinoma with NUT gene changes; mouth cancer; multiple endocrine neoplasia syndromes; multiple myeloma/plasma cell neoplasms; myelodysplastic syndromes (e.g., myelodysplastic/myeloproliferative neoplasms); nasal cavity and paranasal sinus cancer; neuroblastoma; non-Hodgkin lymphoma; oral cancer (e.g., lip and oral cavity cancer, oropharyngeal cancer); osteosarcoma and malignant fibrous histiocytoma of bone; ovarian cancer; pancreatic cancer; pancreatic neuroendocrine tumors (e.g., islet cell tumors); paraganglioma; paranasal sinus and nasal cavity cancer; parathyroid cancer; penile cancer; pharyngeal cancer; pheochromocytoma; pituitary tumor; plasma cell neoplasm/multiple myeloma; pleuropulmonary blastoma; pregnancy and breast cancer; primary peritoneal cancer; prostate cancer; rectal cancer; recurrent cancer; retinoblastoma; salivary gland cancer; sarcoma (e.g. childhood rhabdomyosarcoma, childhood vascular tumors, Ewing sarcoma, Kaposi sarcoma, osteosarcoma (bone cancer), soft tissue sarcoma, uterine sarcoma); skin cancer; small intestine cancer; squamous cell carcinoma of the skin; testicular cancer; throat cancer (e.g. nasopharyngeal cancer, oropharyngeal cancer, hypopharyngeal cancer); thymoma and thymic carcinoma; thyroid cancer; transitional cell cancer of the renal pelvis and ureter; urethral cancer; vaginal cancer; vascular tumors; vulvar cancer; and Wilms tumor and other childhood kidney tumors.

In some embodiments, a cancer is a solid tumor, a hematologic cancer, or both. In some embodiments, a cancer is a solid tumor. In some embodiments, a cancer is a hematologic cancer. In some embodiments, a cancer is a B-cell lymphoma. In some embodiments, a cancer is a lung cancer.

In some embodiments, a subject is diagnosed with a cancer. In some embodiments, a subject is suspected of having a cancer.

In some embodiments, a subject is a mammal. In some embodiments, a subject is a non-human mammal. As used herein, the term “subject” includes humans, domestic animals, such as laboratory animals (e.g., dogs, monkeys, pigs, rats, mice, etc.), household pets (e.g., cats, dogs, rabbits, etc.), livestock (e.g., pigs, cattle, sheep, goats, horses, etc.), and non-domestic animals. In some embodiments, a subject is a human. In some embodiments, a subject (e.g., human) is male. In some embodiments a subject (e.g., human) is female. In some embodiments, a subject is an intersex human. In some embodiments, a human subject: has physiological and/or genetic characteristics associated with one or more sexes; has undergone or received medical interventions that affect physiological characteristics associated with one or more sexes; and/or is intersex. In some embodiments, the sex of a subject is undefined, unknown, or unclear. In some embodiments, a subject has two X chromosomes. In some embodiments, a subject has one X chromosome and one Y chromosome. In some embodiments, a subject has two or more X chromosomes. In some embodiments, a subject has one or more X chromosomes. In some embodiments, a subject has one X chromosome. In some embodiments, a subject has one or more Y chromosomes.

In some embodiments, a subject (e.g., a human) is a child (e.g., birth to 17 years of age). In some embodiments, a subject (e.g., a human) is an adult (18-64 years of age). In some embodiments, a subject (e.g., a human) is an older adult (65 years of age or older).

In some embodiments, a subject (e.g., a human) is at least about 1 year of age, for example, at least about: 2, 3, 4, 5, 6, 7, 8, 9, 10, 11, 12, 15, 18, 19, 20, 25, 30, 35, 40, 45, or 50 years of age. In some embodiments, a subject is at least 14 years of age. In some embodiments, a subject is at least 18 years of age.

In some embodiments, a subject (e.g., a human) is about 90 years of age or younger, for example, about: 85, 80, 75, 70, 65, 60, 55, 50, 45, 40, 35, 30, 29, 25, 24, 23, 22, 20, 18, 17, 16, 15, 13, 11, 10, 7, or 6 years of age or younger.

In some embodiments, a subject is about: 1-7, 2-16, 3-10, 3-11, 3-17, 3-18, 3-23, 5-11, 5-13, 5-23, 5-35, 5-50, 6-18, 6-40, 7-16, 8-18, 8-45, 10-17, 10-23, 10-45, 12-18, 12-29, 12-40, 12-45, 12-50, 13-22, 13-29, 13-35, 13-40, 14-50, 14-100, 15-55, 18-24, 18-45, 18-50, 18-60, 18-65, 18-100, 30-80, or 50-85 years of age.

In some embodiments, a subject is infused with cells disclosed herein (e.g., immune effector cells or mammalian cells disclosed herein). In some embodiments, cells (e.g., immune effector cells or mammalian cells) are autologous. In some embodiments, cells (e.g., immune effector cells or mammalian cells) are syngeneic. In some embodiments, cells (e.g., immune effector cells or mammalian cells) are allogeneic.

Also provided herein are methods of delivering a protein, an organelle, or a combination thereof into a target cell, said methods comprise contacting the target cell with an effective dosage of cells disclosed herein (e.g., immune effector cells or mammalian cells disclosed herein).

In some embodiments, a target cell is a mammalian cell.

In some embodiments, a method delivers one or more proteins into a target cell. Non-limiting example of cells (e.g., immune effector cells or mammalian cells) in which a protein is enriched include dendritic cells (enriched in proteins such as MHC Class II) and T cells (enriched in proteins such as T-cell receptors).

In some embodiments, a method delivers an organelle into a target cell. In some embodiments, an organelle is mitochondria. In some embodiments, a method restores mitochondrial function. In some embodiments, a method is used to treat one or more mitochondrial diseases (See, e.g., Schapira et al., cited as Reference 50, herein).

### Screening Methods

Also provided herein are methods of screening a library of peptide major histocompatibility complex (pMHC) antigens expressed on the surfaces of a population of mammalian cells, said methods comprising:
a) co-culturing the population of mammalian cells with a population of fusogen-expressing T cells,
b) identifying a fused cell produced by fusion of a cell from the population of mammalian cells and a cell from the population of T cells, and
c) identifying the pMHC antigen expressed on the surface of the fused cell.

In some embodiments, a pMHC antigen is a tumor antigen, a pathogenic antigen, a bacterial antigen, or a viral antigen. In some embodiments, a pMHC antigen is a tumor antigen.

In some embodiments, a population of T cells is a population of patient T cells, and a population of mammalian cells is a population of tumor cells from a subject (e.g., a human patient). In some embodiments, a method further comprises identifying a T cell receptor (TCR) expressed on the surface of a fused cell.

Also provided herein are methods of screening a library of fusogens useful for mediating cell fusion, said methods comprising:
a) expressing the library of fusogens in a population of mammalian cells, wherein each mammalian cell expresses a unique fusogen;
b) co-culturing the population of mammalian cells with a population of target cells,
c) identifying a fused cell produced by fusion of a mammalian cell of the population of mammalian cells and a target cell of the population of target cells, and
d) identifying the exogenous fusogen expressed in the fused cell.

In some embodiments, the population of mammalian cells and the population of target cells are co-cultured under suitable conditions, for example, using proliferative cell media. In some embodiments, proliferative cell media comprises human serum. In some embodiments, proliferative cell media lacks human serum. In some embodiments, proliferative cell media comprises bovine serum. In some embodiments, proliferative cell media lacks bovine serum. In some embodiments, proliferative cell media comprises antibiotics. In some embodiments, proliferative cell media lacks antibiotics.

### Fused Cells/Methods of Fusion

Also provided herein are fused cells generated by fusion between a target cell and an immune effector cell or mammalian cell disclosed herein.

Also provided herein are methods of fusing a target cell and an immune effector cell or a mammalian cell, comprising co-culturing the target cell with an immune effector cell or mammalian cell disclosed herein.

In some embodiments, a target cell is an immortal cell. In some embodiments, a target cell is a cancer cell. In some embodiments, a cancer cell is a B-cell lymphoma cell. In some embodiments, a cancer cell is a lung cancer cell.

In some embodiments, a target cell is a neuron, an oligodendrocyte, a microglial cell, a neural stem cell, a hematopoietic stem cell, or a pancreatic beta cell. In some embodiments, a target cell is an antibody-producing B lymphocyte.

Compositions Associated with Engineered Fusogens

Also provided herein are fusogens comprising:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
or any combination of the foregoing.

Also provided herein are fusogens comprising:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC,
or any combination of the foregoing.

Also provided herein are fusogens comprising:
a) an extracellular domain of a viral fusogen derived from a BRV, a BroV, an ARV, an AqRV-A, a RRV, or a variant thereof,
b) a transmembrane domain of a viral fusogen derived from a PsRV, a MdRV, an ARV, a RRV, or a variant thereof,
c) an intracellular domain of a viral fusogen derived from a RRV, an AqRV-C, a MdRV, a Nelson Bay virus, a MaRV, an ARVch, or a variant thereof,
or any combination of the foregoing.

Also provided herein are polynucleotides encoding a fusogen comprising:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, or a HuRVB,
or any combination of the foregoing.

Also provided herein are polynucleotides encoding a fusogen comprising:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC,
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC,
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a VC,
or any combination of the foregoing.

Also provided herein are polynucleotides encoding a fusogen comprising:
a) an extracellular domain of a viral fusogen derived from a BRV, a BroV, an ARV, an AqRV-A, a RRV, or a variant thereof,
b) a transmembrane domain of a viral fusogen derived from a PsRV, a MdRV, an ARV, a RRV, or a variant thereof,
c) an intracellular domain of a viral fusogen derived from a RRV, an AqRV-C, a MdRV, a Nelson Bay virus, a MaRV, an ARVch, or a variant thereof,
or any combination of the foregoing.

In some embodiments, a fusogen comprises an amino acid sequence having at least about 50% sequence identity to at least one sequence set forth in SEQ ID NOs:21-53, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to at least one sequence set forth in SEQ ID NOs:21-53. In some embodiments, a fusogen comprises an amino acid sequence having at least about 80% sequence identity to at least one sequence set forth in SEQ ID NOs:21-53. In some embodiments, a fusogen comprises an amino acid sequence having at least about 90% sequence identity to at least one sequence set forth in SEQ ID NOs:21-53. In some embodiments, a fusogen comprises an amino acid sequence having at least about 95% sequence identity to at least one sequence set forth in SEQ ID NOs:21-53. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to at least one sequence set forth in SEQ ID NOs:21-53.

In some embodiments, a fusogen comprises at least one amino acid addition, deletion, and/or substitution (e.g., at least one conservative substitution such as highly conservative amino acid substitution), relative to a fusogen sequence set forth in any one of SEQ ID NOs:21-53. For example, the number of amino acid addition, deletion, and/or substitutions can be at least about: 2, 3, 4, 5, 6, 7, 8, 9, 10, 11, 12, 13, 14, 15, 16, 17, 18, 19, 20, 21 or 22, or about: 1-22, 1-20, 2-20, 2-18, 3-18, 3-16, 4-16, 4-14, 5-14, 5-12, 6-12, 6-10, 7-10 or 7-8.

In some embodiments, a fusogen comprises an amino acid sequence having 100% sequence identity to a sequence set forth in SEQ ID NOs:21-53.

In some embodiments, a fusogen comprises:
a) an extracellular domain of a viral fusogen derived from a BRV or a variant thereof,
b) a transmembrane domain of a viral fusogen derived from a PsRV or a variant thereof, and
c) an intracellular domain of a viral fusogen derived from an RRV or a variant thereof.

In some embodiments, a fusogen comprises an amino acid sequence having at least about 50% sequence identity to SEQ ID NO:21, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:21. In some embodiments, a fusogen comprises an amino acid sequence having at least about 80% sequence identity to SEQ ID NO:21. In some embodiments, a fusogen comprises an amino acid sequence having at least about 90% sequence identity to SEQ ID NO:21. In some embodiments, a fusogen comprises an amino acid sequence having at least about 95% sequence identity to SEQ ID NO:21. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:21.

In some embodiments, a fusogen comprises:
a) an extracellular domain of a viral fusogen derived from a BRV,
b) a transmembrane domain of a viral fusogen derived from a PsRV, and
c) an intracellular domain of a viral fusogen derived from an RRV.

In some embodiments, a fusogen comprises an amino acid sequence having 100% sequence identity to SEQ ID NO:21.

In some embodiments, a fusogen comprises:
a) an extracellular domain of a viral fusogen derived from a BroV or a variant thereof,
b) a transmembrane domain of a viral fusogen derived from a PsRV or a variant thereof, and
c) an intracellular domain of a viral fusogen derived from a MdRV or a variant thereof.

In some embodiments, a fusogen comprises an amino acid sequence having at least about 50% sequence identity to SEQ ID NO:22, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:22. In some embodiments, a fusogen comprises an amino acid sequence having at least about 80% sequence identity to SEQ ID NO:22. In some embodiments, a fusogen comprises an amino acid sequence having at least about 90% sequence identity to SEQ ID NO:22. In some embodiments, a fusogen comprises an amino acid sequence having at least about 95% sequence identity to SEQ ID NO:22. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:22.

In some embodiments, a fusogen comprises:
a) an extracellular domain of a viral fusogen derived from a BroV,
b) a transmembrane domain of a viral fusogen derived from a PsRV, and
c) an intracellular domain of a viral fusogen derived from a MdRV.

In some embodiments, a fusogen comprises an amino acid sequence having 100% sequence identity to SEQ ID NO:22.

In some embodiments, a fusogen comprises:
a) an extracellular domain of a viral fusogen derived from an ARV or a variant thereof,
b) a transmembrane domain of a viral fusogen derived from a MdRV or a variant thereof, and
c) an intracellular domain of a viral fusogen derived from a Nelson Bay virus or a variant thereof.

In some embodiments, a fusogen comprises an amino acid sequence having at least about 50% sequence identity to SEQ ID NO:23, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:23. In some embodiments, a fusogen comprises an amino acid sequence having at least about 80% sequence identity to SEQ ID NO:23. In some embodiments, a fusogen comprises an amino acid sequence having at least about 90% sequence identity to SEQ ID NO:23. In some embodiments, a fusogen comprises an amino acid sequence having at least about 95% sequence identity to SEQ ID NO:23. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:23.

In some embodiments, a fusogen comprises:
a) an extracellular domain of a viral fusogen derived from an ARV,
b) a transmembrane domain of a viral fusogen derived from a MdRV, and
c) an intracellular domain of a viral fusogen derived from a Nelson Bay virus.

In some embodiments, a fusogen comprises an amino acid sequence having 100% sequence identity to SEQ ID NO:23.

In some embodiments, a fusogen comprises:
a) an extracellular domain of a viral fusogen derived from a BroV or a variant thereof,
b) a transmembrane domain of a viral fusogen derived from a MdRV or a variant thereof, and
c) an intracellular domain of a viral fusogen derived from a MaRV or a variant thereof.

In some embodiments, a fusogen comprises an amino acid sequence having at least about 50% sequence identity to SEQ ID NO:24, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:24. In some embodiments, a fusogen comprises an amino acid sequence having at least about 80% sequence identity to SEQ ID NO:24. In some embodiments, a fusogen comprises an amino acid sequence having at least about 90% sequence identity to SEQ ID NO:24. In some embodiments, a fusogen comprises an amino acid sequence having at least about 95% sequence identity to SEQ ID NO:24. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:24.

In some embodiments, a fusogen comprises:
a) an extracellular domain of a viral fusogen derived from a BroV,
b) a transmembrane domain of a viral fusogen derived from a MdRV, and
c) an intracellular domain of a viral fusogen derived from a MaRV.

In some embodiments, a fusogen comprises an amino acid sequence having 100% sequence identity to SEQ ID NO:24.

In some embodiments, a fusogen comprises:
a) an extracellular domain of a viral fusogen derived from an ARV or a variant thereof,
b) a transmembrane domain of a viral fusogen derived from a MdRV or a variant thereof, and
c) an intracellular domain of a viral fusogen derived from a Nelson Bay virus or a variant thereof.

In some embodiments, a fusogen comprises an amino acid sequence having at least about 50% sequence identity to SEQ ID NO:25, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:25. In some embodiments, a fusogen comprises an amino acid sequence having at least about 80% sequence identity to SEQ ID NO:25. In some embodiments, a fusogen comprises an amino acid sequence having at least about 90% sequence identity to SEQ ID NO:25. In some embodiments, a fusogen comprises an amino acid sequence having at least about 95% sequence identity to SEQ ID NO:25. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:25.

In some embodiments, a fusogen comprises:
a) an extracellular domain of a viral fusogen derived from an ARV,
b) a transmembrane domain of a viral fusogen derived from a MdRV, and
c) an intracellular domain of a viral fusogen derived from a Nelson Bay virus.

In some embodiments, a fusogen comprises an amino acid sequence having 100% sequence identity to SEQ ID NO:25.

In some embodiments, a fusogen comprises:
a) an extracellular domain of a viral fusogen derived from an AqRV-A or a variant thereof,
b) a transmembrane domain of a viral fusogen derived from an ARV or a variant thereof, and
c) an intracellular domain of a viral fusogen derived from a MdRV or a variant thereof.

In some embodiments, a fusogen comprises an amino acid sequence having at least about 50% sequence identity to SEQ ID NO:26, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:26. In some embodiments, a fusogen comprises an amino acid sequence having at least about 80% sequence identity to SEQ ID NO:26. In some embodiments, a fusogen comprises an amino acid sequence having at least about 90% sequence identity to SEQ ID NO:26. In some embodiments, a fusogen comprises an amino acid sequence having at least about 95% sequence identity to SEQ ID NO:26. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:26.

In some embodiments, a fusogen comprises:
a) an extracellular domain of a viral fusogen derived from an AqRV-A,
b) a transmembrane domain of a viral fusogen derived from an ARV, and
c) an intracellular domain of a viral fusogen derived from a MdRV.

In some embodiments, a fusogen comprises an amino acid sequence having 100% sequence identity to SEQ ID NO:26.

In some embodiments, a fusogen comprises:
a) an extracellular domain of a viral fusogen derived from an ARV or a variant thereof,
b) a transmembrane domain of a viral fusogen derived from a PsRV or a variant thereof, and
c) an intracellular domain of a viral fusogen derived from an ARV or a variant thereof.

In some embodiments, a fusogen comprises an amino acid sequence having at least about 50% sequence identity to SEQ ID NO:27, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:27. In some embodiments, a fusogen comprises an amino acid sequence having at least about 80% sequence identity to SEQ ID NO:27. In some embodiments, a fusogen comprises an amino acid sequence having at least about 90% sequence identity to SEQ ID NO:27. In some embodiments, a fusogen comprises an amino acid sequence having at least about 95% sequence identity to SEQ ID NO:27. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:27.

In some embodiments, a fusogen comprises:
a) an extracellular domain of a viral fusogen derived from an ARV,
b) a transmembrane domain of a viral fusogen derived from a PsRV, and
c) an intracellular domain of a viral fusogen derived from an ARV.

In some embodiments, a fusogen comprises an amino acid sequence having 100% sequence identity to SEQ ID NO:27.

In some embodiments, a fusogen comprises:
a) an extracellular domain of a viral fusogen derived from a halibut reovirus or a variant thereof,
b) a transmembrane domain of a viral fusogen derived from a RRVtes or a variant thereof, and
c) an intracellular domain of a viral fusogen derived from an ARV or a variant thereof.

In some embodiments, a fusogen comprises an amino acid sequence having at least about 50% sequence identity to SEQ ID NO:28, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:28. In some embodiments, a fusogen comprises an amino acid sequence having at least about 80% sequence identity to SEQ ID NO:28. In some embodiments, a fusogen comprises an amino acid sequence having at least about 90% sequence identity to SEQ ID NO:28. In some embodiments, a fusogen comprises an amino acid sequence having at least about 95% sequence identity to SEQ ID NO:28. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:28.

In some embodiments, a fusogen comprises:
a) an extracellular domain of a viral fusogen derived from a halibut reovirus,
b) a transmembrane domain of a viral fusogen derived from a RRVtes, and
c) an intracellular domain of a viral fusogen derived from an ARV.

In some embodiments, a fusogen comprises an amino acid sequence having 100% sequence identity to SEQ ID NO:28.

In some embodiments, a fusogen comprises:
a) an extracellular domain of a viral fusogen derived from a MaRV or a variant thereof,
b) a transmembrane domain of a viral fusogen derived from a Nelson Bay virus or a variant thereof, and
c) an intracellular domain of a viral fusogen derived from an ARV or a variant thereof.

In some embodiments, a fusogen comprises an amino acid sequence having at least about 50% sequence identity to SEQ ID NO:29, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:29. In some embodiments, a fusogen comprises an amino acid sequence having at least about 80% sequence identity to SEQ ID NO:29. In some embodiments, a fusogen comprises an amino acid sequence having at least about 90% sequence identity to SEQ ID NO:29. In some embodiments, a fusogen comprises an amino acid sequence having at least about 95% sequence identity to SEQ ID NO:29. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:29.

In some embodiments, a fusogen comprises:
a) an extracellular domain of a viral fusogen derived from a MaRV,
b) a transmembrane domain of a viral fusogen derived from a Nelson Bay virus, and
c) an intracellular domain of a viral fusogen derived from an ARV.

In some embodiments, a fusogen comprises an amino acid sequence having 100% sequence identity to SEQ ID NO:29.

In some embodiments, a fusogen comprises:
a) an extracellular domain of a viral fusogen derived from a BroV or a variant thereof,
b) a transmembrane domain of a viral fusogen derived from a BroV or a variant thereof, and
c) an intracellular domain of a viral fusogen derived from an ARV or a variant thereof.

In some embodiments, a fusogen comprises an amino acid sequence having at least about 50% sequence identity to SEQ ID NO:30, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:30. In some embodiments, a fusogen comprises an amino acid sequence having at least about 80% sequence identity to SEQ ID NO:30. In some embodiments, a fusogen comprises an amino acid sequence having at least about 90% sequence identity to SEQ ID NO:30. In some embodiments, a fusogen comprises an amino acid sequence having at least about 95% sequence identity to SEQ ID NO:30. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:30.

In some embodiments, a fusogen comprises:
a) an extracellular domain of a viral fusogen derived from a BroV,
b) a transmembrane domain of a viral fusogen derived from a BroV, and
c) an intracellular domain of a viral fusogen derived from an ARV.

In some embodiments, a fusogen comprises an amino acid sequence having 100% sequence identity to SEQ ID NO:30.

In some embodiments, a fusogen comprises:
a) an extracellular domain of a viral fusogen derived from a BroV or a variant thereof,
b) a transmembrane domain of a viral fusogen derived from a BroV or a variant thereof, and
c) an intracellular domain of a viral fusogen derived from a PsRV or a variant thereof.

In some embodiments, a fusogen comprises an amino acid sequence having at least about 50% sequence identity to SEQ ID NO:31, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:31. In some embodiments, a fusogen comprises an amino acid sequence having at least about 80% sequence identity to SEQ ID NO:31. In some embodiments, a fusogen comprises an amino acid sequence having at least about 90% sequence identity to SEQ ID NO:31. In some embodiments, a fusogen comprises an amino acid sequence having at least about 95% sequence identity to SEQ ID NO:31. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:31.

In some embodiments, a fusogen comprises:
a) an extracellular domain of a viral fusogen derived from a BroV,
b) a transmembrane domain of a viral fusogen derived from a BroV, and
c) an intracellular domain of a viral fusogen derived from a PsRV.

In some embodiments, a fusogen comprises an amino acid sequence having 100% sequence identity to SEQ ID NO:31.

In some embodiments, a fusogen comprises:
a) an extracellular domain of a viral fusogen derived from a BroV or a variant thereof,
b) a transmembrane domain of a viral fusogen derived from a Nelson Bay virus or a variant thereof, and
c) an intracellular domain of a viral fusogen derived from an ARV or a variant thereof.

In some embodiments, a fusogen comprises an amino acid sequence having at least about 50% sequence identity to SEQ ID NO:32, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:32. In some embodiments, a fusogen comprises an amino acid sequence having at least about 80% sequence identity to SEQ ID NO:32. In some embodiments, a fusogen comprises an amino acid sequence having at least about 90% sequence identity to SEQ ID NO:32. In some embodiments, a fusogen comprises an amino acid sequence having at least about 95% sequence identity to SEQ ID NO:32. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:32.

In some embodiments, a fusogen comprises:
a) an extracellular domain of a viral fusogen derived from a BroV,
b) a transmembrane domain of a viral fusogen derived from a Nelson Bay virus, and
c) an intracellular domain of a viral fusogen derived from an ARV.

In some embodiments, a fusogen comprises an amino acid sequence having 100% sequence identity to SEQ ID NO:32.

In some embodiments, a fusogen comprises:
a) an extracellular domain of a viral fusogen derived from a BroV or a variant thereof,
b) a transmembrane domain of a viral fusogen derived from a Nelson Bay virus or a variant thereof, and
c) an intracellular domain of a viral fusogen derived from a PsRV or a variant thereof.

In some embodiments, a fusogen comprises an amino acid sequence having at least about 50% sequence identity to SEQ ID NO:33, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:33. In some embodiments, a fusogen comprises an amino acid sequence having at least about 80% sequence identity to SEQ ID NO:33. In some embodiments, a fusogen comprises an amino acid sequence having at least about 90% sequence identity to SEQ ID NO:33. In some embodiments, a fusogen comprises an amino acid sequence having at least about 95% sequence identity to SEQ ID NO:33. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:33.

In some embodiments, a fusogen comprises:
a) an extracellular domain of a viral fusogen derived from a BroV,
b) a transmembrane domain of a viral fusogen derived from a Nelson Bay virus, and c) an intracellular domain of a viral fusogen derived from a PsRV.

In some embodiments, a fusogen comprises an amino acid sequence having 100% sequence identity to SEQ ID NO:33.

In some embodiments, a fusogen comprises:
a) an extracellular domain of a viral fusogen derived from a BroV or a variant thereof,
b) a transmembrane domain of a viral fusogen derived from a VC or a variant thereof, and
c) an intracellular domain of a viral fusogen derived from a Nelson Bay virus or a variant thereof.

In some embodiments, a fusogen comprises an amino acid sequence having at least about 50% sequence identity to SEQ ID NO:34, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:34. In some embodiments, a fusogen comprises an amino acid sequence having at least about 80% sequence identity to SEQ ID NO:34. In some embodiments, a fusogen comprises an amino acid sequence having at least about 90% sequence identity to SEQ ID NO:34. In some embodiments, a fusogen comprises an amino acid sequence having at least about 95% sequence identity to SEQ ID NO:34. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:34.

In some embodiments, a fusogen comprises:
a) an extracellular domain of a viral fusogen derived from a BroV,
b) a transmembrane domain of a viral fusogen derived from a VC, and
c) an intracellular domain of a viral fusogen derived from a Nelson Bay virus.

In some embodiments, a fusogen comprises an amino acid sequence having 100% sequence identity to SEQ ID NO:34.

In some embodiments, a fusogen comprises:
a) an extracellular domain of a viral fusogen derived from a BroV or a variant thereof,
b) a transmembrane domain of a viral fusogen derived from a RRV or a variant thereof, and
c) an intracellular domain of a viral fusogen derived from a MdRV or a variant thereof.

In some embodiments, a fusogen comprises an amino acid sequence having at least about 50% sequence identity to SEQ ID NO:35, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:35. In some embodiments, a fusogen comprises an amino acid sequence having at least about 80% sequence identity to SEQ ID NO:35. In some embodiments, a fusogen comprises an amino acid sequence having at least about 90% sequence identity to SEQ ID NO:35. In some embodiments, a fusogen comprises an amino acid sequence having at least about 95% sequence identity to SEQ ID NO:35. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:35.

In some embodiments, a fusogen comprises:
a) an extracellular domain of a viral fusogen derived from a BroV,
b) a transmembrane domain of a viral fusogen derived from a RRV, and
c) an intracellular domain of a viral fusogen derived from a MdRV.

In some embodiments, a fusogen comprises an amino acid sequence having 100% sequence identity to SEQ ID NO:35.

In some embodiments, a fusogen comprises:
a) an extracellular domain of a viral fusogen derived from a BroV or a variant thereof,
b) a transmembrane domain of a viral fusogen derived from a RRV or a variant thereof, and
c) an intracellular domain of a viral fusogen derived from a Nelson Bay virus or a variant thereof.

In some embodiments, a fusogen comprises an amino acid sequence having at least about 50% sequence identity to SEQ ID NO:36, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:36. In some embodiments, a fusogen comprises an amino acid sequence having at least about 80% sequence identity to SEQ ID NO:36. In some embodiments, a fusogen comprises an amino acid sequence having at least about 90% sequence identity to SEQ ID NO:36. In some embodiments, a fusogen comprises an amino acid sequence having at least about 95% sequence identity to SEQ ID NO:36. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:36.

In some embodiments, a fusogen comprises:
a) an extracellular domain of a viral fusogen derived from a BroV,
b) a transmembrane domain of a viral fusogen derived from a RRV, and
c) an intracellular domain of a viral fusogen derived from a Nelson Bay virus.

In some embodiments, a fusogen comprises an amino acid sequence having 100% sequence identity to SEQ ID NO:36.

In some embodiments, a fusogen comprises:
a) an extracellular domain of a viral fusogen derived from a BroV or a variant thereof,
b) a transmembrane domain of a viral fusogen derived from a RRVtes or a variant thereof, and
c) an intracellular domain of a viral fusogen derived from an ARV or a variant thereof.

In some embodiments, a fusogen comprises an amino acid sequence having at least about 50% sequence identity to SEQ ID NO:37, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:37. In some embodiments, a fusogen comprises an amino acid sequence having at least about 80% sequence identity to SEQ ID NO:37. In some embodiments, a fusogen comprises an amino acid sequence having at least about 90% sequence identity to SEQ ID NO:37. In some embodiments, a fusogen comprises an amino acid sequence having at least about 95% sequence identity to SEQ ID NO:37. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:37.

In some embodiments, a fusogen comprises:
a) an extracellular domain of a viral fusogen derived from a BroV,
b) a transmembrane domain of a viral fusogen derived from a RRVtes, and
c) an intracellular domain of a viral fusogen derived from an ARV.

In some embodiments, a fusogen comprises an amino acid sequence having 100% sequence identity to SEQ ID NO:37.

In some embodiments, a fusogen comprises:
a) an extracellular domain of a viral fusogen derived from a BroV or a variant thereof,
b) a transmembrane domain of a viral fusogen derived from a RRVtes or a variant thereof, and
c) an intracellular domain of a viral fusogen derived from an ARVtu or a variant thereof.

In some embodiments, a fusogen comprises an amino acid sequence having at least about 50% sequence identity to SEQ ID NO:38, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:38. In some embodiments, a fusogen comprises an amino acid sequence having at least about 80% sequence identity to SEQ ID NO:38. In some embodiments, a fusogen comprises an amino acid sequence having at least about 90% sequence identity to SEQ ID NO:38. In some embodiments, a fusogen comprises an amino acid sequence having at least about 95% sequence identity to SEQ ID NO:38. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:38.

In some embodiments, a fusogen comprises:
a) an extracellular domain of a viral fusogen derived from a BroV,
b) a transmembrane domain of a viral fusogen derived from a RRVtes, and
c) an intracellular domain of a viral fusogen derived from an ARVtu.

In some embodiments, a fusogen comprises an amino acid sequence having 100% sequence identity to SEQ ID NO:38.

In some embodiments, a fusogen comprises:
a) an extracellular domain of a viral fusogen derived from a BRV or a variant thereof,
b) a transmembrane domain of a viral fusogen derived from a MdRV or a variant thereof, and
c) an intracellular domain of a viral fusogen derived from a MdRV or a variant thereof.

In some embodiments, a fusogen comprises an amino acid sequence having at least about 50% sequence identity to SEQ ID NO:39, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:39. In some embodiments, a fusogen comprises an amino acid sequence having at least about 80% sequence identity to SEQ ID NO:39. In some embodiments, a fusogen comprises an amino acid sequence having at least about 90% sequence identity to SEQ ID NO:39. In some embodiments, a fusogen comprises an amino acid sequence having at least about 95% sequence identity to SEQ ID NO:39. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:39.

In some embodiments, a fusogen comprises:
a) an extracellular domain of a viral fusogen derived from a BRV,
b) a transmembrane domain of a viral fusogen derived from a MdRV, and
c) an intracellular domain of a viral fusogen derived from a MdRV.

In some embodiments, a fusogen comprises an amino acid sequence having 100% sequence identity to SEQ ID NO:39.

In some embodiments, a fusogen comprises:
a) an extracellular domain of a viral fusogen derived from a BRV or a variant thereof,
b) a transmembrane domain of a viral fusogen derived from a BRV or a variant thereof, and
c) an intracellular domain of a viral fusogen derived from a PsRV or a variant thereof.

In some embodiments, a fusogen comprises an amino acid sequence having at least about 50% sequence identity to SEQ ID NO:40, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:40. In some embodiments, a fusogen comprises an amino acid sequence having at least about 80% sequence identity to SEQ ID NO:40. In some embodiments, a fusogen comprises an amino acid sequence having at least about 90% sequence identity to SEQ ID NO:40. In some embodiments, a fusogen comprises an amino acid sequence having at least about 95% sequence identity to SEQ ID NO:40. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:40.

In some embodiments, a fusogen comprises:
a) an extracellular domain of a viral fusogen derived from a BRV,
b) a transmembrane domain of a viral fusogen derived from a BRV, and
c) an intracellular domain of a viral fusogen derived from a PsRV.

In some embodiments, a fusogen comprises an amino acid sequence having 100% sequence identity to SEQ ID NO:40.

In some embodiments, a fusogen comprises:
a) an extracellular domain of a viral fusogen derived from a BRV or a variant thereof,
b) a transmembrane domain of a viral fusogen derived from a Nelson Bay virus or a variant thereof, and
c) an intracellular domain of a viral fusogen derived from a PsRV or a variant thereof.

In some embodiments, a fusogen comprises an amino acid sequence having at least about 50% sequence identity to SEQ ID NO:41, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:41. In some embodiments, a fusogen comprises an amino acid sequence having at least about 80% sequence identity to SEQ ID NO:41. In some embodiments, a fusogen comprises an amino acid sequence having at least about 90% sequence identity to SEQ ID NO:41. In some embodiments, a fusogen comprises an amino acid sequence having at least about 95% sequence identity to SEQ ID NO:41. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:41.

In some embodiments, a fusogen comprises:
a) an extracellular domain of a viral fusogen derived from a BRV,
b) a transmembrane domain of a viral fusogen derived from a Nelson Bay virus, and
c) an intracellular domain of a viral fusogen derived from a PsRV.

In some embodiments, a fusogen comprises an amino acid sequence having 100% sequence identity to SEQ ID NO:41.

In some embodiments, a fusogen comprises:
a) an extracellular domain of a viral fusogen derived from a BRV or a variant thereof,
b) a transmembrane domain of a viral fusogen derived from a RRVtes or a variant thereof, and
c) an intracellular domain of a viral fusogen derived from an ARVtu or a variant thereof.

In some embodiments, a fusogen comprises an amino acid sequence having at least about 50% sequence identity to SEQ ID NO:42, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:42. In some embodiments, a fusogen comprises an amino acid sequence having at least about 80% sequence identity to SEQ ID NO:42. In some embodiments, a fusogen comprises an amino acid sequence having at least about 90% sequence identity to SEQ ID NO:42. In some embodiments, a fusogen comprises an amino acid sequence having at least about 95% sequence identity to SEQ ID NO:42. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:42.

In some embodiments, a fusogen comprises:
a) an extracellular domain of a viral fusogen derived from a BRV,
b) a transmembrane domain of a viral fusogen derived from a RRVtes, and
c) an intracellular domain of a viral fusogen derived from an ARVtu.

In some embodiments, a fusogen comprises an amino acid sequence having 100% sequence identity to SEQ ID NO:42.

In some embodiments, a fusogen comprises:
a) an extracellular domain of a viral fusogen derived from a BRV or a variant thereof,
b) a transmembrane domain of a viral fusogen derived from a RRVtes or a variant thereof, and
c) an intracellular domain of a viral fusogen derived from an ARV or a variant thereof.

In some embodiments, a fusogen comprises an amino acid sequence having at least about 50% sequence identity to SEQ ID NO:43, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:43. In some embodiments, a fusogen comprises an amino acid sequence having at least about 80% sequence identity to SEQ ID NO:43. In some embodiments, a fusogen comprises an amino acid sequence having at least about 90% sequence identity to SEQ ID NO:43. In some embodiments, a fusogen comprises an amino acid sequence having at least about 95% sequence identity to SEQ ID NO:43. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:43.

In some embodiments, a fusogen comprises:
a) an extracellular domain of a viral fusogen derived from a BRV,
b) a transmembrane domain of a viral fusogen derived from a RRVtes, and
c) an intracellular domain of a viral fusogen derived from an ARV.

In some embodiments, a fusogen comprises an amino acid sequence having 100% sequence identity to SEQ ID NO:43.

In some embodiments, a fusogen comprises:
a) an extracellular domain of a viral fusogen derived from a AqRV-C or a variant thereof,
b) a transmembrane domain of a viral fusogen derived from an ARV or a variant thereof, and
c) an intracellular domain of a viral fusogen derived from an ARV or a variant thereof.

In some embodiments, a fusogen comprises an amino acid sequence having at least about 50% sequence identity to SEQ ID NO:44, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:44. In some embodiments, a fusogen comprises an amino acid sequence having at least about 80% sequence identity to SEQ ID NO:44. In some embodiments, a fusogen comprises an amino acid sequence having at least about 90% sequence identity to SEQ ID NO:44. In some embodiments, a fusogen comprises an amino acid sequence having at least about 95% sequence identity to SEQ ID NO:44. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:44.

In some embodiments, a fusogen comprises:
a) an extracellular domain of a viral fusogen derived from a AqRV-C,
b) a transmembrane domain of a viral fusogen derived from an ARV, and
c) an intracellular domain of a viral fusogen derived from an ARV.

In some embodiments, a fusogen comprises an amino acid sequence having 100% sequence identity to SEQ ID NO:44.

In some embodiments, a fusogen comprises:
a) an extracellular domain of a viral fusogen derived from an AqRV-C or a variant thereof,
b) a transmembrane domain of a viral fusogen derived from a RRV or a variant thereof, and
c) an intracellular domain of a viral fusogen derived from an ARV or a variant thereof.

In some embodiments, a fusogen comprises an amino acid sequence having at least about 50% sequence identity to SEQ ID NO:45, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:45. In some embodiments, a fusogen comprises an amino acid sequence having at least about 80% sequence identity to SEQ ID NO:45. In some embodiments, a fusogen comprises an amino acid sequence having at least about 90% sequence identity to SEQ ID NO:45. In some embodiments, a fusogen comprises an amino acid sequence having at least about 95% sequence identity to SEQ ID NO:45. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:45.

In some embodiments, a fusogen comprises:
a) an extracellular domain of a viral fusogen derived from an AqRV-C,
b) a transmembrane domain of a viral fusogen derived from a RRV, and
c) an intracellular domain of a viral fusogen derived from an ARV.

In some embodiments, a fusogen comprises an amino acid sequence having 100% sequence identity to SEQ ID NO:45.

In some embodiments, a fusogen comprises:
a) an extracellular domain of a viral fusogen derived from an AqRV-A or a variant thereof,
b) a transmembrane domain of a viral fusogen derived from a MaRV or a variant thereof, and
c) an intracellular domain of a viral fusogen derived from an ARVtu or a variant thereof.

In some embodiments, a fusogen comprises an amino acid sequence having at least about 50% sequence identity to SEQ ID NO:46, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:46. In some embodiments, a fusogen comprises an amino acid sequence having at least about 80% sequence identity to SEQ ID NO:46. In some embodiments, a fusogen comprises an amino acid sequence having at least about 90% sequence identity to SEQ ID NO:46. In some embodiments, a fusogen comprises an amino acid sequence having at least about 95% sequence identity to SEQ ID NO:46. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:46.

In some embodiments, a fusogen comprises:
a) an extracellular domain of a viral fusogen derived from an AqRV-A,
b) a transmembrane domain of a viral fusogen derived from a MaRV, and
c) an intracellular domain of a viral fusogen derived from an ARVtu.

In some embodiments, a fusogen comprises an amino acid sequence having 100% sequence identity to SEQ ID NO:46.

In some embodiments, a fusogen comprises:
a) an extracellular domain of a viral fusogen derived from an AqRV-A or a variant thereof,
b) a transmembrane domain of a viral fusogen derived from a BRV or a variant thereof, and
c) an intracellular domain of a viral fusogen derived from a MdRV or a variant thereof.

In some embodiments, a fusogen comprises an amino acid sequence having at least about 50% sequence identity to SEQ ID NO:47, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:47. In some embodiments, a fusogen comprises an amino acid sequence having at least about 80% sequence identity to SEQ ID NO:47. In some embodiments, a fusogen comprises an amino acid sequence having at least about 90% sequence identity to SEQ ID NO:47. In some embodiments, a fusogen comprises an amino acid sequence having at least about 95% sequence identity to SEQ ID NO:47. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:47.

In some embodiments, a fusogen comprises:
a) an extracellular domain of a viral fusogen derived from an AqRV-A,
b) a transmembrane domain of a viral fusogen derived from a BRV, and
c) an intracellular domain of a viral fusogen derived from a MdRV.

In some embodiments, a fusogen comprises an amino acid sequence having 100% sequence identity to SEQ ID NO:47.

In some embodiments, a fusogen comprises:
a) an extracellular domain of a viral fusogen derived from a Nelson Bay virus or a variant thereof,
b) a transmembrane domain of a viral fusogen derived from an ARV or a variant thereof, and
c) an intracellular domain of a viral fusogen derived from a PsRV or a variant thereof.

In some embodiments, a fusogen comprises an amino acid sequence having at least about 50% sequence identity to SEQ ID NO:48, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:48. In some embodiments, a fusogen comprises an amino acid sequence having at least about 80% sequence identity to SEQ ID NO:48. In some embodiments, a fusogen comprises an amino acid sequence having at least about 90% sequence identity to SEQ ID NO:48. In some embodiments, a fusogen comprises an amino acid sequence having at least about 95% sequence identity to SEQ ID NO:48. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:48.

In some embodiments, a fusogen comprises:
a) an extracellular domain of a viral fusogen derived from a Nelson Bay virus, b) a transmembrane domain of a viral fusogen derived from an ARV, and
c) an intracellular domain of a viral fusogen derived from a PsRV.

In some embodiments, a fusogen comprises an amino acid sequence having 100% sequence identity to SEQ ID NO:48.

In some embodiments, a fusogen comprises:
a) an extracellular domain of a viral fusogen derived from a Nelson Bay virus or a variant thereof,
b) a transmembrane domain of a viral fusogen derived from an ARV or a variant thereof, and
c) an intracellular domain of a viral fusogen derived from an ARV or a variant thereof.

In some embodiments, a fusogen comprises an amino acid sequence having at least about 50% sequence identity to SEQ ID NO:49, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:49. In some embodiments, a fusogen comprises an amino acid sequence having at least about 80% sequence identity to SEQ ID NO:49. In some embodiments, a fusogen comprises an amino acid sequence having at least about 90% sequence identity to SEQ ID NO:49. In some embodiments, a fusogen comprises an amino acid sequence having at least about 95% sequence identity to SEQ ID NO:49. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:49.

In some embodiments, a fusogen comprises:
a) an extracellular domain of a viral fusogen derived from a Nelson Bay virus,
b) a transmembrane domain of a viral fusogen derived from an ARV, and
c) an intracellular domain of a viral fusogen derived from an ARV.

In some embodiments, a fusogen comprises an amino acid sequence having 100% sequence identity to SEQ ID NO:49.

In some embodiments, a fusogen comprises:
a) an extracellular domain of a viral fusogen derived from a Nelson Bay virus or a variant thereof,
b) a transmembrane domain of a viral fusogen derived from a BroV or a variant thereof, and
c) an intracellular domain of a viral fusogen derived from an ARV or a variant thereof.

In some embodiments, a fusogen comprises an amino acid sequence having at least about 50% sequence identity to SEQ ID NO:50, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:50. In some embodiments, a fusogen comprises an amino acid sequence having at least about 80% sequence identity to SEQ ID NO:50. In some embodiments, a fusogen comprises an amino acid sequence having at least about 90% sequence identity to SEQ ID NO:50. In some embodiments, a fusogen comprises an amino acid sequence having at least about 95% sequence identity to SEQ ID NO:50. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:50.

In some embodiments, a fusogen comprises:
a) an extracellular domain of a viral fusogen derived from a Nelson Bay virus,
b) a transmembrane domain of a viral fusogen derived from a BroV, and
c) an intracellular domain of a viral fusogen derived from an ARV.

In some embodiments, a fusogen comprises an amino acid sequence having 100% sequence identity to SEQ ID NO:50.

In some embodiments, a fusogen comprises:
a) an extracellular domain of a viral fusogen derived from a Nelson Bay virus or a variant thereof,
b) a transmembrane domain of a viral fusogen derived from an AqRV-A or a variant thereof, and
c) an intracellular domain of a viral fusogen derived from a PsRV or a variant thereof.

In some embodiments, a fusogen comprises an amino acid sequence having at least about 50% sequence identity to SEQ ID NO:51, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:51. In some embodiments, a fusogen comprises an amino acid sequence having at least about 80% sequence identity to SEQ ID NO:51. In some embodiments, a fusogen comprises an amino acid sequence having at least about 90% sequence identity to SEQ ID NO:51. In some embodiments, a fusogen comprises an amino acid sequence having at least about 95% sequence identity to SEQ ID NO:51. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:51.

In some embodiments, a fusogen comprises:
a) an extracellular domain of a viral fusogen derived from a Nelson Bay virus or a variant thereof,
b) a transmembrane domain of a viral fusogen derived from an AqRV-A or a variant thereof, and
c) an intracellular domain of a viral fusogen derived from a PsRV or a variant thereof.

In some embodiments, a fusogen comprises an amino acid sequence having 100% sequence identity to SEQ ID NO:51.

In some embodiments, a fusogen comprises:
a) an extracellular domain of a viral fusogen derived from a VC or a variant thereof,
b) a transmembrane domain of a viral fusogen derived from a PsRV or a variant thereof, and
c) an intracellular domain of a viral fusogen derived from a MdRV or a variant thereof.

In some embodiments, a fusogen comprises an amino acid sequence having at least about 50% sequence identity to SEQ ID NO:52, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:52. In some embodiments, a fusogen comprises an amino acid sequence having at least about 80% sequence identity to SEQ ID NO:52. In some embodiments, a fusogen comprises an amino acid sequence having at least about 90% sequence identity to SEQ ID NO:52. In some embodiments, a fusogen comprises an amino acid sequence having at least about 95% sequence identity to SEQ ID NO:52. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:52.

In some embodiments, a fusogen comprises:
a) an extracellular domain of a viral fusogen derived from a VC,
b) a transmembrane domain of a viral fusogen derived from a PsRV, and
c) an intracellular domain of a viral fusogen derived from a MdRV.

In some embodiments, a fusogen comprises an amino acid sequence having 100% sequence identity to SEQ ID NO:52.

In some embodiments, a fusogen comprises:
a) an extracellular domain of a viral fusogen derived from a VC or a variant thereof,
b) a transmembrane domain of a viral fusogen derived from a RRVtes or a variant thereof, and
c) an intracellular domain of a viral fusogen derived from a Nelson Bay virus or a variant thereof.

In some embodiments, a fusogen comprises an amino acid sequence having at least about 50% sequence identity to SEQ ID NO:53, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:53. In some embodiments, a fusogen comprises an amino acid sequence having at least about 80% sequence identity to SEQ ID NO:53. In some embodiments, a fusogen comprises an amino acid sequence having at least about 90% sequence identity to SEQ ID NO:53. In some embodiments, a fusogen comprises an amino acid sequence having at least about 95% sequence identity to SEQ ID NO:53. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:53.

In some embodiments, a fusogen comprises:
a) an extracellular domain of a viral fusogen derived from a VC,
b) a transmembrane domain of a viral fusogen derived from a RRVtes, and
c) an intracellular domain of a viral fusogen derived from a Nelson Bay virus.

In some embodiments, a fusogen comprises an amino acid sequence having 100% sequence identity to SEQ ID NO:53.

In some embodiments, a fusogen comprises an amino acid sequence having at least about 50% sequence identity to SEQ ID NO: 112, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:112. In some embodiments, a fusogen comprises an amino acid sequence having at least about 80% sequence identity to SEQ ID NO:112. In some embodiments, a fusogen comprises an amino acid sequence having at least about 90% sequence identity to SEQ ID NO:112. In some embodiments, a fusogen comprises an amino acid sequence having at least about 95% sequence identity to SEQ ID NO:112. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:112.

In some embodiments, a fusogen comprises an amino acid sequence having at least about 50% sequence identity to SEQ ID NO:113, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:113. In some embodiments, a fusogen comprises an amino acid sequence having at least about 80% sequence identity to SEQ ID NO:113. In some embodiments, a fusogen comprises an amino acid sequence having at least about 90% sequence identity to SEQ ID NO:113. In some embodiments, a fusogen comprises an amino acid sequence having at least about 95% sequence identity to SEQ ID NO:113. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 113.

In some embodiments, a fusogen comprises an amino acid sequence having at least about 50% sequence identity to SEQ ID NO:114, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:114. In some embodiments, a fusogen comprises an amino acid sequence having at least about 80% sequence identity to SEQ ID NO:114. In some embodiments, a fusogen comprises an amino acid sequence having at least about 90% sequence identity to SEQ ID NO: 114. In some embodiments, a fusogen comprises an amino acid sequence having at least about 95% sequence identity to SEQ ID NO:114. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 114.

In some embodiments, a fusogen comprises an amino acid sequence having at least about 50% sequence identity to SEQ ID NO: 115, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:115. In some embodiments, a fusogen comprises an amino acid sequence having at least about 80% sequence identity to SEQ ID NO:115. In some embodiments, a fusogen comprises an amino acid sequence having at least about 90% sequence identity to SEQ ID NO:115. In some embodiments, a fusogen comprises an amino acid sequence having at least about 95% sequence identity to SEQ ID NO:115. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:115.

In some embodiments, a fusogen comprises an amino acid sequence having at least about 50% sequence identity to SEQ ID NO:116, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:116. In some embodiments, a fusogen comprises an amino acid sequence having at least about 80% sequence identity to SEQ ID NO:116. In some embodiments, a fusogen comprises an amino acid sequence having at least about 90% sequence identity to SEQ ID NO:116. In some embodiments, a fusogen comprises an amino acid sequence having at least about 95% sequence identity to SEQ ID NO:116. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:116.

In some embodiments, a fusogen comprises an amino acid sequence having at least about 50% sequence identity to SEQ ID NO:117, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:117. In some embodiments, a fusogen comprises an amino acid sequence having at least about 80% sequence identity to SEQ ID NO:117. In some embodiments, a fusogen comprises an amino acid sequence having at least about 90% sequence identity to SEQ ID NO:117. In some embodiments, a fusogen comprises an amino acid sequence having at least about 95% sequence identity to SEQ ID NO:117. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 117.

In some embodiments, a fusogen comprises an amino acid sequence having at least about 50% sequence identity to SEQ ID NO: 118, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:118. In some embodiments, a fusogen comprises an amino acid sequence having at least about 80% sequence identity to SEQ ID NO:118. In some embodiments, a fusogen comprises an amino acid sequence having at least about 90% sequence identity to SEQ ID NO:118. In some embodiments, a fusogen comprises an amino acid sequence having at least about 95% sequence identity to SEQ ID NO:118. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:118.

In some embodiments, a fusogen comprises an amino acid sequence having at least about 50% sequence identity to SEQ ID NO: 119, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:119. In some embodiments, a fusogen comprises an amino acid sequence having at least about 80% sequence identity to SEQ ID NO:119. In some embodiments, a fusogen comprises an amino acid sequence having at least about 90% sequence identity to SEQ ID NO:119. In some embodiments, a fusogen comprises an amino acid sequence having at least about 95% sequence identity to SEQ ID NO:119. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:119.

In some embodiments, a fusogen comprises an amino acid sequence having at least about 50% sequence identity to SEQ ID NO: 120, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:120. In some embodiments, a fusogen comprises an amino acid sequence having at least about 80% sequence identity to SEQ ID NO:120. In some embodiments, a fusogen comprises an amino acid sequence having at least about 90% sequence identity to SEQ ID NO: 120. In some embodiments, a fusogen comprises an amino acid sequence having at least about 95% sequence identity to SEQ ID NO:120. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 120.

In some embodiments, a fusogen comprises an amino acid sequence having at least about 50% sequence identity to SEQ ID NO: 121, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:121. In some embodiments, a fusogen comprises an amino acid sequence having at least about 80% sequence identity to SEQ ID NO:121. In some embodiments, a fusogen comprises an amino acid sequence having at least about 90% sequence identity to SEQ ID NO: 121. In some embodiments, a fusogen comprises an amino acid sequence having at least about 95% sequence identity to SEQ ID NO:121. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 121.

In some embodiments, a fusogen comprises an amino acid sequence having at least about 50% sequence identity to SEQ ID NO: 122, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:122. In some embodiments, a fusogen comprises an amino acid sequence having at least about 80% sequence identity to SEQ ID NO: 122. In some embodiments, a fusogen comprises an amino acid sequence having at least about 90% sequence identity to SEQ ID NO:122. In some embodiments, a fusogen comprises an amino acid sequence having at least about 95% sequence identity to SEQ ID NO:122. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:122.

In some embodiments, a fusogen comprises an amino acid sequence having at least about 50% sequence identity to SEQ ID NO: 123, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:123. In some embodiments, a fusogen comprises an amino acid sequence having at least about 80% sequence identity to SEQ ID NO:123. In some embodiments, a fusogen comprises an amino acid sequence having at least about 90% sequence identity to SEQ ID NO: 123. In some embodiments, a fusogen comprises an amino acid sequence having at least about 95% sequence identity to SEQ ID NO:123. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:123.

In some embodiments, a fusogen comprises an amino acid sequence having at least about 50% sequence identity to SEQ ID NO: 124, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:124. In some embodiments, a fusogen comprises an amino acid sequence having at least about 80% sequence identity to SEQ ID NO:124. In some embodiments, a fusogen comprises an amino acid sequence having at least about 90% sequence identity to SEQ ID NO: 124. In some embodiments, a fusogen comprises an amino acid sequence having at least about 95% sequence identity to SEQ ID NO:124. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:124.

In some embodiments, a fusogen comprises an amino acid sequence having at least about 50% sequence identity to SEQ ID NO: 125, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:125. In some embodiments, a fusogen comprises an amino acid sequence having at least about 80% sequence identity to SEQ ID NO:125. In some embodiments, a fusogen comprises an amino acid sequence having at least about 90% sequence identity to SEQ ID NO: 125. In some embodiments, a fusogen comprises an amino acid sequence having at least about 95% sequence identity to SEQ ID NO:125. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 125.

In some embodiments, a fusogen comprises an amino acid sequence having at least about 50% sequence identity to SEQ ID NO:126, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:126. In some embodiments, a fusogen comprises an amino acid sequence having at least about 80% sequence identity to SEQ ID NO: 126. In some embodiments, a fusogen comprises an amino acid sequence having at least about 90% sequence identity to SEQ ID NO:126. In some embodiments, a fusogen comprises an amino acid sequence having at least about 95% sequence identity to SEQ ID NO: 126. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:126.

In some embodiments, a fusogen comprises an amino acid sequence having at least about 50% sequence identity to SEQ ID NO:127, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:127. In some embodiments, a fusogen comprises an amino acid sequence having at least about 80% sequence identity to SEQ ID NO: 127. In some embodiments, a fusogen comprises an amino acid sequence having at least about 90% sequence identity to SEQ ID NO: 127. In some embodiments, a fusogen comprises an amino acid sequence having at least about 95% sequence identity to SEQ ID NO: 127. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 127.

In some embodiments, a fusogen comprises an amino acid sequence having at least about 50% sequence identity to SEQ ID NO: 128, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:128. In some embodiments, a fusogen comprises an amino acid sequence having at least about 80% sequence identity to SEQ ID NO:128. In some embodiments, a fusogen comprises an amino acid sequence having at least about 90% sequence identity to SEQ ID NO: 128. In some embodiments, a fusogen comprises an amino acid sequence having at least about 95% sequence identity to SEQ ID NO: 128. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 128.

In some embodiments, a fusogen comprises an amino acid sequence having at least about 50% sequence identity to SEQ ID NO: 129, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:129. In some embodiments, a fusogen comprises an amino acid sequence having at least about 80% sequence identity to SEQ ID NO:129. In some embodiments, a fusogen comprises an amino acid sequence having at least about 90% sequence identity to SEQ ID NO: 129. In some embodiments, a fusogen comprises an amino acid sequence having at least about 95% sequence identity to SEQ ID NO:129. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 129.

In some embodiments, a fusogen comprises an amino acid sequence having at least about 50% sequence identity to SEQ ID NO: 130, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:130. In some embodiments, a fusogen comprises an amino acid sequence having at least about 80% sequence identity to SEQ ID NO:130. In some embodiments, a fusogen comprises an amino acid sequence having at least about 90% sequence identity to SEQ ID NO:130. In some embodiments, a fusogen comprises an amino acid sequence having at least about 95% sequence identity to SEQ ID NO:130. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 130.

In some embodiments, a fusogen comprises an amino acid sequence having at least about 50% sequence identity to SEQ ID NO:131, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:131. In some embodiments, a fusogen comprises an amino acid sequence having at least about 80% sequence identity to SEQ ID NO:131. In some embodiments, a fusogen comprises an amino acid sequence having at least about 90% sequence identity to SEQ ID NO: 131. In some embodiments, a fusogen comprises an amino acid sequence having at least about 95% sequence identity to SEQ ID NO:131. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 131.

In some embodiments, a fusogen comprises an amino acid sequence having at least about 50% sequence identity to SEQ ID NO: 132, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:132. In some embodiments, a fusogen comprises an amino acid sequence having at least about 80% sequence identity to SEQ ID NO:132. In some embodiments, a fusogen comprises an amino acid sequence having at least about 90% sequence identity to SEQ ID NO: 132. In some embodiments, a fusogen comprises an amino acid sequence having at least about 95% sequence identity to SEQ ID NO: 132. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 132.

In some embodiments, a fusogen comprises an amino acid sequence having at least about 50% sequence identity to SEQ ID NO: 133, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:133. In some embodiments, a fusogen comprises an amino acid sequence having at least about 80% sequence identity to SEQ ID NO:133. In some embodiments, a fusogen comprises an amino acid sequence having at least about 90% sequence identity to SEQ ID NO: 133. In some embodiments, a fusogen comprises an amino acid sequence having at least about 95% sequence identity to SEQ ID NO:133. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 133.

In some embodiments, a fusogen comprises an amino acid sequence having at least about 50% sequence identity to SEQ ID NO:134, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:134. In some embodiments, a fusogen comprises an amino acid sequence having at least about 80% sequence identity to SEQ ID NO:134. In some embodiments, a fusogen comprises an amino acid sequence having at least about 90% sequence identity to SEQ ID NO: 134. In some embodiments, a fusogen comprises an amino acid sequence having at least about 95% sequence identity to SEQ ID NO:134. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:134.

In some embodiments, a fusogen comprises an amino acid sequence having at least about 50% sequence identity to SEQ ID NO:135, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:135. In some embodiments, a fusogen comprises an amino acid sequence having at least about 80% sequence identity to SEQ ID NO:135. In some embodiments, a fusogen comprises an amino acid sequence having at least about 90% sequence identity to SEQ ID NO: 135. In some embodiments, a fusogen comprises an amino acid sequence having at least about 95% sequence identity to SEQ ID NO: 135. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO: 135.

In some embodiments, a fusogen comprises an amino acid sequence having at least about 50% sequence identity to SEQ ID NO: 136, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:136. In some embodiments, a fusogen comprises an amino acid sequence having at least about 80% sequence identity to SEQ ID NO: 136. In some embodiments, a fusogen comprises an amino acid sequence having at least about 90% sequence identity to SEQ ID NO: 136. In some embodiments, a fusogen comprises an amino acid sequence having at least about 95% sequence identity to SEQ ID NO:136. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:136.

In some embodiments, a fusogen comprises an amino acid sequence having at least about 50% sequence identity to SEQ ID NO:137, for example, having at least about: 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to SEQ ID NO:137. In some embodiments, a fusogen comprises an amino acid sequence having at least about 80% sequence identity to SEQ ID NO:137. In some embodiments, a fusogen comprises an amino acid sequence having at least about 90% sequence identity to SEQ ID NO:137. In some embodiments, a fusogen comprises an amino acid sequence having at least about 95% sequence identity to SEQ ID NO:137. In some embodiments, a fusogen comprises an amino acid sequence having about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99% sequence identity to SEQ ID NO:137.

### EXAMPLE EMBODIMENTS

1. An immune effector cell comprising a nucleotide sequence encoding an exogenous fusogen.

2. The immune effector cell of Embodiment 1, wherein the immune effector cell is a T cell, a monocyte, a B cell, a natural killer (NK) cell, a macrophage, or a dendritic cell.

3. The immune effector cell of Embodiment 1 or 2, wherein the immune effector cell is a T cell.

4. The immune effector cell of any one of Embodiments 1-3, wherein the immune effector cell is a helper CD4⁺ T cell, a cytotoxic CD8⁺ T cell, a memory T cell, a regulatory CD4⁺ T cell, an innate-like T cell, a natural killer T cell, a mucosal associated invariant T cell, or a Gamma Delta T cell.

5. The immune effector cell of any one of Embodiments 1-4, wherein the immune effector cell is a helper CD4⁺ T cell, a cytotoxic CD8⁺ T cell, a memory T cell, or a regulatory CD4⁺ T cell.

6. The immune effector cell of Embodiment 1 or 2, wherein the immune effector cell is a monocyte.

7. The immune effector cell of any one of Embodiments 1, 2 and 6, wherein the immune effector cell is a CD14⁺+CD16″ monocyte, a CD14⁺CD16⁺⁺ monocyte, or a CD14⁺+CD16⁺ monocyte.

8. The immune effector cell of Embodiment 1 or 2, wherein the immune effector cell is a B cell.

9. The immune effector cell of any one of Embodiments 1, 2 and 8, wherein the immune effector cell is a plasmablast, a plasma cell, a lymphoplasmacytoid cell, a memory B cell, a B-2 cell, a B-1 cell, or a regulatory B cell (Breg).

10. The immune effector cell of Embodiment 1 or 2, wherein the immune effector cell is a natural killer (NK) cell.

11. The immune effector cell of Embodiment 10, wherein the NK cell is CD56^{BRIGHT}

12. The immune effector cell of Embodiment 10, wherein the NK cell is CD56^{DIM}

13. The immune effector cell of Embodiment 1 or 2, wherein the immune effector cell is a macrophage.

14. The immune effector cell of any one of Embodiments 1, 2 and 13, wherein the immune effector cell is an adipose tissue macrophage, a monocyte, a Kupffer cell, a sinus histiocyte, an alveolar macrophage (dust cell), a tissue macrophage (histiocyte) leading to giant cell, a microglial cell, a Hofbauer cell, an intraglomerular mesangial cell, an osteoclast, a Langerhans cell, an epithelioid cell, a red pulp macrophage (sinusoidal lining cell), a peritoneal macrophage, a lysomac, or a perivascular macrophage.

15. The immune effector cell of any one of Embodiments 1-14, wherein the immune effector cell is a human immune cell.

16. The immune effector cell of any one of Embodiments 1-14, wherein the immune effector cell is a humanized immune cell.

17. A mammalian cell, comprising a nucleotide sequence encoding a cell-targeting molecule and a nucleotide sequence encoding an exogenous fusogen.

18. The immune effector cell of any one of Embodiments 1-16, or the mammalian cell of Embodiment 17, wherein the exogenous fusogen is a viral fusogen or a fragment thereof.

19. The immune effector cell of any one of Embodiments 1-16 and 18, or the mammalian cell of Embodiment 17 or 18, wherein the exogenous fusogen is about 50 to about 1,000 amino acids in length.

20. The immune effector cell of any one of Embodiments 1-16, 18, and 19, or the mammalian cell of any one of Embodiments 17-19, wherein the exogenous fusogen is about 50 to about 250 amino acids in length.

21. The immune effector cell of any one of Embodiments 1-16 and 18-20, or the mammalian cell of any one of Embodiments 17-20, wherein the exogenous fusogen is encoded by an avian orthoreovirus (ARV), a Nelson Bay virus, a Broome orthoreovirus (BroV), a reptilian orthoreovirus (RRV), a Baboon orthoreovirus (BRV), a group C aquareovirus (AqRV-C), a group A aquareovirus (AqRV-A), a halibut reovirus, a Mahlapitisi orthoreovirus (MaRV), a Muscovy duck orthoreovirus (MdRV), a reptilian Testudine orthoreovirus (RRVtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaceous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), or a human rotavirus species B NSP-1 (HuRVB), or a variant thereof.

22. The immune effector cell of any one of Embodiments 1-16 and 18-21, or the mammalian cell of any one of Embodiments 17-21, wherein the exogenous fusogen comprises an amino acid sequence having at least 50% sequence identity to at least one sequence set forth in SEQ ID NOs: 1-20.

23. The immune effector cell of any one of Embodiments 1-16 and 18-22, or the mammalian cell of any one of Embodiments 17-22, wherein the exogenous fusogen comprises an amino acid sequence having 100% sequence identity to a sequence set forth in SEQ ID NOs:1-20.

24 The immune effector cell of any one of Embodiments 1-16 and 18-20, or the mammalian cell of any one of Embodiments 17-20, wherein the exogenous fusogen comprises:
a) an extracellular domain encoded by an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof,
b) a transmembrane domain encoded by an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, or a variant thereof, or
c) an intracellular domain encoded by an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PORVB, a HuRVB, or a variant thereof, or any combination of the foregoing.

25. The immune effector cell of any one of Embodiments 1-16, 18-20, and 24, or the mammalian cell of any one of Embodiments 17-20, and 24, wherein the exogenous fusogen comprises:
a) an extracellular domain encoded by a BRV, a BroV, an ARV, an AqRV-A, a RRV, or a variant thereof,
b) a transmembrane domain encoded by a PsRV, a MdRV, a RRV, an ARV, or a variant thereof,
c) an intracellular domain encoded by a RRV, an AqRV-C, a MdRV, a Nelson Bay virus, a MaRV, an ARVch, or a variant thereof, or any combination of the foregoing.

26. The immune effector cell of any one of Embodiments 1-16, 18-20, 24, and 25, or the mammalian cell of any one of Embodiments 17-20, 24, and 25, wherein the exogenous fusogen comprises an amino acid sequence having at least 50% sequence identity to at least one sequence set forth in SEQ ID NOs:21-53.

27. The immune effector cell of any one of Embodiments 1-16, 18-20, and 24-26, or the mammalian cell of any one of Embodiments 17-20 and 24-26, wherein the exogenous fusogen comprises an amino acid sequence having 100% sequence identity to at least one sequence set forth in SEQ ID NOs:21-53.

28. The immune effector cell of any one of Embodiments 1-16 and 18-27, or the mammalian cell of any one of Embodiments 17-27, wherein the immune effector cell expresses the exogenous fusogen.

29. The immune effector cell of any one of Embodiments 1-16 and 18-28, or the mammalian cell of any one of Embodiments 17-28, wherein the level of the exogenous fusogen in the immune effector cell or the mammalian cell is conditionally inducible or suppressible.

30. The immune effector cell or the mammalian cell of Embodiment 29, wherein the level of the exogenous fusogen is regulated by a small molecule, a light-switchable system, a ligand-switchable system, or a combination thereof.

31. The immune effector cell or the mammalian cell of Embodiment 30, wherein:
a) the small molecule comprises tetracycline (TET), doxycycline (DOX), caffeine, 4-hydroxytamoxifen, estrogen, ecdysone, abscisic acid, mifepristone, xylose, FKBP12-rapamycin, an HCV NS3/4A protease inhibitor, or a combination thereof, optionally wherein the HCV NS3/4A protease inhibitor is grazoprevir,
b) the light-switchable system comprises Phytochrome B (PhyB) and phytochrome interaction factor 6 (PIF6),
c) the light-switchable system comprises GIGANTEA and the light oxygen voltage (LOV) domain of Flavin-Binding Kelch Repeat F-Box 1 (FKF1), or
d) the ligand-switchable system comprises a synthetic Notch receptor (synNotch), a modular extracellular sensor, or a synthetic intramembrane proteolysis receptor (SNIPR),
or any combination of the foregoing.

32. The immune effector cell or the mammalian cell of Embodiment 30 or 31, wherein the small molecule is tetracycline (TET) or doxycycline (DOX).

33. The immune effector cell of any one of Embodiments 1-16 and 18-32, further comprising a nucleotide sequence encoding a cell-targeting molecule.

34. The immune effector cell of Embodiment 33 or the mammalian cell of any one of Embodiments 17-32, wherein the cell-targeting molecule recognizes a cell-surface marker for one or more cancer cells, neurons, oligodendrocytes, microglial cells, neural stem cells, hematopoietic stem cells, astrocytes, or pancreatic beta cells.

35. The immune effector cell or the mammalian cell of Embodiment 34, wherein the cell-targeting molecule recognizes:
L1 cell adhesion molecule (LICAM), neurexin 3, vesicular glutamate transporter 1 (VGLUT1), vesicular inhibitory amino acid transporter (VIAAT), neuroligin 1, neuroligin 2, neural cell adhesion molecule 1 (NCAM1), vesicular acetylcholine transporter (VAChT), folate receptor-1 (FOLR1), gamma-aminobutyric acid B receptor 1 (GABA(b)R1), GABA(b)R2, glutamate ionotropic receptor NMDA type subunit 1 (GRIN1), GRIN2B, or solute carrier family 6 member 4 (SLC6A4),
neural/glial antigen 2 (NG2), oligodendrocyte marker O1, oligodendrocyte marker 04, A2B5, or myelin-oligodendrocyte glycoprotein (MOG),
P2Y12, macrophage colony-stimulating factor receptor (M-CSFR), or CX3C motif chemokine receptor 1 (CX3CR1),
CD133 or CD49F,
stem cell antigen-1 (Sca-1), CD27, CD34, CD38, CD43, CD117, or CD150,
A2B5, connexin 43, or aquaporin-4 (AQP-4), CD71 or CD24,
B-cell activating factor (BAFF) receptor, B-cell maturation antigen (BCMA),
CD3, CD5, CD19, CD20, CD22, CD28, CD30, CD38, CD79B, or programmed cell death protein 1 (PD-1), or
AXL, B7 homolog 3 protein (B7-H3), carcinoembryonic antigen (CEA), CD70, claudin18.2 (CLDN18.2), delta-like ligand 3 (DLL3), disialoganglioside (GD2), epidermal growth factor receptor (EGFR), glypican-3 (GPC3), guanylyl cyclase C (GUCY2C), human epidermal growth factor receptor 2 (HER2), Kita-Kyushu lung cancer antigen-1 (KK-LC-1), Lewis Y (LEY), mesothelin (MSLN), MUCIN 1 (MUC1), NEW YORK esophageal squamous cell carcinoma 1 (NY-ESO-1), positive programmed death-ligand 1 (PD-L1), prostate specific membrane antigen (PSMA), prostate stem cell antigen (PSCA), receptor-tyrosine-kinase like orphan receptor 1 (ROR1), transforming growth factor beta (TGF-β), Kirsten rat sarcoma virus (KRAS) G12D, melanoma antigen recognized by T cells 1 (MART-1), melanoma-associated antigen 3 (MAGE-A3), tumor protein p53 (TP53), FMS-like tyrosine kinase 3 (FLT3), or alkaline phosphatase placental-like 2 (ALPPL2).

36. The immune effector cell of any one of Embodiments 33-35, or the mammalian cell of any one of Embodiments 17-32, 34, and 35, wherein the cell-targeting molecule recognizes a cell-surface marker for cancer cells.

37. The immune effector cell or the mammalian cell of Embodiment 36, wherein the cell-surface marker comprises a B-cell lymphoma-derived antigen.

38. The immune effector cell or the mammalian cell of Embodiment 36 or 37, wherein the cell-surface marker comprises B-cell activating factor (BAFF) receptor, B-cell maturation antigen (BCMA), CD3, CD5, CD19, CD20, CD22, CD28, CD30, CD38, CD79B, or programmed cell death protein 1 (PD-1).

39. The immune effector cell or the mammalian cell of Embodiment 36, wherein the cell-surface marker comprises a lung cancer-derived antigen.

40. The immune effector cell or the mammalian cell of Embodiment 36 or 39, wherein the cell-surface marker comprises AXL, B7 homolog 3 protein (B7-H3), carcinoembryonic antigen (CEA), CD70, claudin18.2 (CLDN18.2), delta-like ligand 3 (DLL3), disialoganglioside (GD2), epidermal growth factor receptor (EGFR), glypican-3 (GPC3), guanylyl cyclase C (GUCY2C), human epidermal growth factor receptor 2 (HER2), Kita-Kyushu lung cancer antigen-1 (KK-LC-1), Lewis Y (LEY), mesothelin (MSLN), MUCIN 1 (MUC1), NEW YORK esophageal squamous cell carcinoma 1 (NY-ESO-1), positive programmed death-ligand 1 (PD-L1), prostate specific membrane antigen (PSMA), prostate stem cell antigen (PSCA), receptor-tyrosine-kinase like orphan receptor 1 (ROR1), transforming growth factor beta (TGF-β), Kirsten rat sarcoma virus (KRAS) G12D, melanoma antigen recognized by T cells 1 (MART-1), melanoma-associated antigen 3 (MAGE-A3), tumor protein p53 (TP53), FMS-like tyrosine kinase 3 (FLT3), or alkaline phosphatase placental-like 2 (ALPPL2).

41. The immune effector cell of any one of Embodiments 33-40, or the mammalian cell of any one of Embodiments 17-32 and 34-40, wherein the cell-targeting molecule comprises a native protein.

42. The immune effector cell of any one of Embodiments 33-41, or the mammalian cell of any one of Embodiments 17-32 and 34-41, wherein the cell-targeting molecule comprises an antibody or an antigen-binding fragment thereof, a T cell receptor (TCR), an integrin, or a cell adhesion molecule.

43. The immune effector cell or the mammalian cell of Embodiment 42, wherein the cell-targeting molecule comprises an integrin.

44. The immune effector cell or the mammalian cell of Embodiment 43, wherein the integrin comprises integrin beta-1 (ITGβ1), integrin alpha-5 (ITGα5), lymphocyte function-associated antigen 1 (LFA-1), or integrin subunit alpha V (ITGαV).

45. The immune effector cell or the mammalian cell of Embodiment 42, wherein the cell-targeting molecule comprises a cell-adhesion molecule.

46. The immune effector cell or the mammalian cell of Embodiment 45, wherein the cell-adhesion molecule comprises intercellular adhesion molecule-1 (ICAM-1), vascular cell adhesion protein-1 (VCAM-1), neural cell adhesion molecule 1 (N-CAM1), or platelet endothelial cell adhesion molecule-1 (PE-CAM1).

47. The immune effector cell or the mammalian cell of Embodiment 42, wherein the cell-targeting molecule comprises a patient-derived TCR.

48. The immune effector cell of any one of Embodiments 33-40, or the mammalian cell of any one of Embodiments 17-32 and 34-40, wherein the cell-targeting molecule comprises an engineered protein.

49. The immune effector cell or the mammalian cell of Embodiment 48, wherein the engineered protein comprises an antibody or an antigen-binding fragment thereof, or an engineered receptor.

50. The immune effector cell or the mammalian cell of Embodiment 49, wherein the engineered receptor comprises a chimeric antigen receptor (CAR), an engineered T cell receptor (TCR), a synthetic Notch receptor (synNotch), a modular extracellular sensor, a synthetic intramembrane proteolysis receptor (SNIPR), or a cell adhesion molecule.

51. The immune effector cell of any one of Embodiments 33-50, or the mammalian cell of any one of Embodiments 17-32 and 34-50, wherein the cell-targeting molecule comprises an anti-CD19 single chain variable fragment (scFv).

52. The immune effector cell or the mammalian cell of Embodiment 51, wherein the cell-targeting molecule further comprises an intracellular adhesion domain.

53. The immune effector cell or the mammalian cell of Embodiment 52, wherein the intracellular adhesion domain comprises an intracellular domain of vascular cell adhesion molecule-1 (VCAM-1), P-Selectin, L-Selectin, MHC class I-restricted T cell-associated molecule (CRTAM), integrin beta2 (ITGβ2), integrin beta1 (ITGβ1), E-Selectin, P-Cadherin, CD103, E-Cadherin, N-Cadherin, mucin-4 (MUC-4), or intercellular adhesion molecule 1 (ICAM1).

54. The immune effector cell of any one of Embodiments 33-53, or the mammalian cell of any one of Embodiments 17-32 and 34-53, wherein the cell-targeting molecule recognizes at least two cell-surface markers.

55. The immune effector cell of any one of Embodiments 33-54, or the mammalian cell of any one of Embodiments 17-32 and 34-54, wherein the level of the cell-targeting molecule is inducible or suppressible.

56. The immune effector cell or the mammalian cell of Embodiment 55, wherein the level of the molecule in the immune effector cell or the mammalian cell is conditionally inducible by a small molecule, a light-switchable system, a ligand-switchable system, or a combination thereof.

57. The immune effector cell of any one of Embodiments 33-56, or the mammalian cell of any one of Embodiments 17-32 and 34-56, further comprising a nucleotide sequence encoding a payload.

58. The immune effector cell or the mammalian cell of Embodiment 57, wherein the payload comprises a therapeutic agent.

59. The immune effector cell or the mammalian cell of Embodiment 57 or 58, wherein the payload comprises a cytokine.

60. The immune effector cell or the mammalian cell of any one of Embodiments 57-59, wherein the payload induces cell death.

61. The immune effector cell or the mammalian cell of any one of Embodiments 57-60, wherein the payload induces apoptosis, pyroptosis, or necroptosis.

62. The immune effector cell or the mammalian cell of Embodiment 57, 58, 60 or 61, wherein the payload comprises a truncated p15 BID (tBID), caspase 1, receptor-interacting protein kinase-3 (RIPK3), caspase-3, caspase-7, caspase-8, caspase-9, mixed lineage kinase domain-like pseudokinase (MLKL), Gasdermin-D (GSDMD), Gasdermin-E (GSDME), or nerve injury-induced protein 1 (Ninj1).

63. The immune effector cell or the mammalian cell of any one of Embodiments 57-59, wherein the payload induces an inflammatory immune response.

64. The immune effector cell or the mammalian cell of any one of Embodiments 57-61, and 63, wherein the payload comprises stimulator of interferon genes (STING), interferon γ, high mobility group box 1 (HMGB1), interleukin-2 (IL-2), IL-12, IL-18, or tumor necrosis factor alpha (TNFα).

65. The immune effector cell or the mammalian cell of any one of Embodiments 57-59, wherein the payload comprises a checkpoint inhibitor.

66. The immune effector cell or the mammalian cell of Embodiment 57, 58 or 65, wherein the payload comprises an antibody that binds to PD-1, PD-L1, CTLA-4, or LAG3.

67. The immune effector cell or the mammalian cell of any one of Embodiments 57-66, wherein the payload is humanized.

68. The immune effector cell or the mammalian cell of any one of Embodiments 57-67, wherein the immune effector cell or the mammalian cell expresses the payload.

69. The immune effector cell or the mammalian cell of any one of Embodiments 57-68, wherein expression and/or activity of the payload is inducible by a small molecule, a light-switchable system, a ligand-switchable system, or a combination thereof.

70. A composition comprising the immune effector cell of any one of Embodiments 1-16 and 18-69 or the mammalian cell of any one of Embodiments 17-32 and 34-69.

71. A pharmaceutical composition comprising the composition of Embodiment 70 and a pharmaceutically acceptable carrier.

72. A kit comprising a container and optionally an instruction for use, wherein the container comprises the composition of Embodiment 70 or the pharmaceutical composition of Embodiment 71.

73. A method of treating cancer in a subject in need thereof, comprising administering to the subject a therapeutically effective amount of the immune effector cell of any one of Embodiments 1-16 and 18-69, the mammalian cell of any one of Embodiments 17-32 and 34-69, the composition of Embodiment 70, or the pharmaceutical composition of Embodiment 71.

74. The immune effector cell of any one of Embodiments 1-16 and 18-69, the mammalian cell of any one of Embodiments 17-32 and 34-69, the composition of Embodiment 70, the pharmaceutical composition of Embodiment 71, or the kit of Embodiment 72, for use in treating cancer in a subject in need thereof.

75. Use of the immune effector cell of any one of Embodiments 1-16 and 18-69, the mammalian cell of any one of Embodiments 17-32 and 34-69, the composition of Embodiment 70, the pharmaceutical composition of Embodiment 71, or the kit of Embodiment 72, for the preparation of a medicament for treating cancer in a subject in need thereof.

76. A method of killing a cancer cell, comprising contacting the cancer cell with an effective dosage of the immune effector cells of any one of Embodiments 1-16 and 18-69, or the mammalian cell of any one of Embodiments 17-32 and 34-69.

77. A method of screening a library of peptide major histocompatibility complex (pMHC) antigens expressed on the surfaces of a population of mammalian cells, comprising:
a) co-culturing the population of mammalian cells with a population of fusogen-expressing T cells,
b) identifying a fused cell produced by fusion of a cell from the population of mammalian cells and a cell from the population of T cells, and
c) identifying the pMHC antigen expressed on the surface of the fused cell.

78. The method of Embodiment 77, wherein the pMHC antigen is a tumor antigen, a pathogenic antigen, a bacterial antigen, or a viral antigen.

79. The method of Embodiment 77 or 78, wherein the pMHC antigen is a tumor antigen.

80. The method of Embodiment 79, wherein the population of T cells is a population of patient T cells, and wherein the population of mammalian cells is a population of tumor cells from the patient.

81. The method of any one of Embodiments 77-80, further comprising identifying a T cell receptor (TCR) expressed on the surface of the fused cell.

82. A method of screening a library of fusogens useful for mediating cell fusion, comprising:
a) expressing the library of fusogens in a population of mammalian cells, wherein each mammalian cell expresses a unique fusogen;
b) co-culturing the population of mammalian cells with a population of target cells,
c) identifying a fused cell produced by fusion of a mammalian cell of the population of mammalian cells and a target cell of the population of target cells, and
d) identifying the exogenous fusogen expressed in the fused cell.

83. A fused cell generated by fusion between a target cell and the immune effector cell of any one of Embodiments 1-16 and 18-69, or the mammalian cell of any one of Embodiments 17-32 and 34-69.

84. A method of fusing a target cell and an immune effector cell or a mammalian cell, comprising co-culturing the target cell with the immune effector cell of any one of Embodiments 1-16 and 18-69, or the mammalian cell of any one of Embodiments 17-32 and 34-69.

85. The fused cell of Embodiment 83, or the method of Embodiment 84, wherein the target cell is an immortal cell.

86. The fused cell of Embodiment 83 or 85 or the method of Embodiment 84 or 85, wherein the target cell is a cancer cell.

87. The fused cell or the method of Embodiment 86, wherein the cancer cell is a B-cell lymphoma cell.

88. The fused cell or the method of Embodiment 86, wherein the cancer cell is a lung cancer cell.

89. The fused cell of Embodiment 83, or the method of Embodiment 84, wherein the target cell is a neuron, an oligodendrocyte, a microglial cell, a neural stem cell, a hematopoietic stem cell, or a pancreatic beta cell.

90. A polypeptide comprising:
a) an extracellular domain encoded by a BRV, a BroV, an ARV, an AqRV-A, or a RRV,
b) a transmembrane domain encoded by a PsRV, a MdRV, a RRV, or an ARV,
c) an intracellular domain encoded by a RRV, an AqRV-C, a MdRV, a Nelson Bay virus, a MaRV, or an ARVch,
or any combination of the foregoing.

91. The polypeptide of Embodiment 90, comprising an amino acid sequence having at least 85% sequence identity to at least one sequence set forth in SEQ ID NOs:21-53.

92. The polypeptide of any Embodiment 90 or 91, comprising an amino acid sequence having 100% sequence identity to a sequence set forth in SEQ ID NOs:21-53.

93. A polynucleotide encoding the polypeptide of any one of Embodiments 90-92.

94. A mammalian cell comprising the polypeptide of any one of Embodiments 90-92 or the polynucleotide of Embodiment 93.

### EXEMPLIFICATION

Lymphoma is a devastating disease which afflicts around 60,000 people per year. Chimeric Antigen Receptor T-cells (CAR-T) treatment has resulted in rare instances of complete remission for certain B-cell lymphomas; however, most patients present with tumor reoccurrence or resistance. Novel pre-clinical treatment strategies for treatment of B-cell lymphoma have encompassed use of immunostimulatory agents to increase immune response and promote durable tumor regression. While beneficial, these strategies lack targetability and control, which limits clinical viability.

CAR-T therapies have reshaped cancer treatment, however currently available therapies suffer from inefficient tumor ablation. As a result, most patients treated with CAR-T exhibit tumor relapse, and often require additional interventions to ameliorate symptoms. Immune cell therapies (ICT) have reshaped the landscape of cancer treatment, evidenced by durable tumor eradication in several cancers¹⁻². However, ICTs such as CART suffer from off-target tumor killing, over-activation or silencing within the tumor microenvironment, and are limited in their controllability once administered³⁻⁵.

The types of engineering efforts in ICT can be generalized into two broad categories: (1) engineering of the T-cell receptor, and (2) augmenting or altering endogenous T-cell signaling pathways through genetic modification. In the first category, engineered T-cell receptors via tumor-specific T-cell receptor (TCR) or synthetic receptors have achieved clinical success in a variety of cancers⁶. There are several barriers to effective response, many of which are due to endogenous T-cell signaling programs or tumor resistance. To circumvent these issues, the second category seeks to re-wire endogenous signaling pathways through receptor control, overriding exhaustion programs, or the addition of exogenous molecules to stimulate anti-tumor T-cell response⁷⁻⁹. Taken together, these efforts seek to optimize endogenous T-cell function; however, novel or non-endogenous T-cell effector functions may be required to achieve durable and multi-cancer responsiveness.

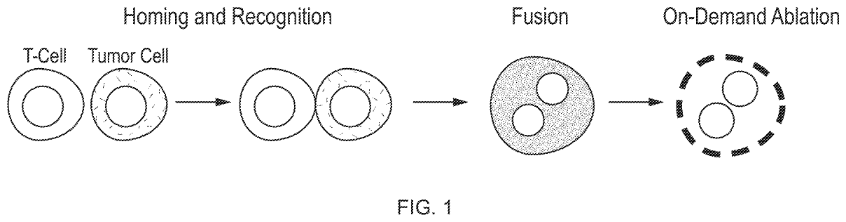

To meet this need, as described in this disclosure, the T-cell immunological synapse was re-engineered towards precise, on-target T-cell-cancer cell fusion (FIG. 1). This novel strategy, Immune Cell Fusion (ICF), permits targeted ablation of cancer cells in an inducible, customizable, and regulatable manner. In contrast to canonical T-cell-cancer cell engagement, which relies upon endogenous signaling for tumor cell death, ICF circumvents endogenous signaling and permits targeted multi-modality cancer cell killing via engineered receptors and synthetic user-defined circuits.

Disclosed herein is a new therapeutic modality, establishing ICF as a new targeted immunotherapy and unlocking a novel delivery platform. ICF is employed to treat cancers, including hematological cancers (e.g., B-cell lymphoma) and solid tumors (e.g., lung cancers). The disclosure includes the adaptation of synthetic receptors and genetic circuits in targeted ICF.

The disclosure includes advancement of ICF T-cell therapy through use of synthetic receptors and gene circuits for ensuring targeted and efficacious T-cell-cancer cell fusion. The disclosure also includes investigation of the genetic basis of cell-cell fusion, for example, through primary T-cell CRISPR screens using a high-throughput fusion assay. In addition, a comprehensive fusogen domain screen is employed to inform design and advanced control over ICF T-cells. Immunostimulatory cell death programs in B-cell and lung tumors are activated and controlled in a targeted manner, using a clinically relevant suite of gene circuits, in conjunction with ICF. ICF-mediated inducible apoptosis, pyroptosis and necroptosis are employed in mouse models of B cell lymphoma and lung cancer to examine both tumor remission and immune response.

### Developing ICF Synthetic Circuits and Receptors for Targeted Tumor Ablation

Synthetic receptors and circuits that activate T-cell-tumor cell fusion upon recognition of a clinically relevant tumor antigen, CD19, were designed and tested. Receptors, circuit design, and fusogens were benchmarked for fusion ability using a high-throughput fusion assay. Viable designs were tested in primary T-cells in vitro, indicating that other synthetic receptors (e.g., to other tumor antigens) and circuits can also be used in ICF.

ICF T-cells permit on-demand, multi-modal cell death ablation of fused tumor cells. Programmable cell death pathways for use in ICF T-cells, using a synthetic zinc finger transcription regulators (SynZiFTR) platform, are designed and tested. ICF T-cells with fusion-dependent tumor ablation switches are augmented to ensure on-target tumor eradication via non-inflammatory and/or inflammatory cell death.

### Determining Intrinsic and Extrinsic Fusogen Signaling in T-Cells

Extrinsic factors of T-cell ICF are determined through use of CRISPR-based gene activation and inhibition screens. Fusogen architecture and function are examined through construction and evaluation of a combinatorial fusogen domain library.

### Assessing ICF-Mediated Immunogenic Cell Death in the Tumor Microenvironment

Both immunoquiescent and immunostimulatory cell death are activated in ICF-targeted CD19⁺ B-cell lymphoma and lung cancer in vivo. The response of the adaptive and innate immune system to apoptosis, pyroptosis and necroptosis are compared via scRNA-seq and Flow Cytometry. Tumor growth is assessed to determine both the tumor and immune response to targeted ICF cell ablation.

### Example 1. Characterization of Cell Fusogens and Fusion

Cell fusion is an essential mechanism, during both homeostasis and disease, that is carried out by fusogens. In homeostasis, the formation of muscle, remodeling of bone, and development of the placenta involve cell-cell fusion¹⁰. In disease, viruses employ fusogens for entry into hosts in order to promote viral proliferation and infection¹¹. Highlighting their high efficiency, viral fusogens have been repurposed for gene and protein delivery in a variety of forms¹². A unique class of fusogens from the non-enveloped Reoviridae family employs FAST (fusogen-associated small transmembrane) proteins, which utilize host cytoskeleton for membrane fusion and viral propagation¹³.

### FAST Proteins Fused Mammalian Membranes

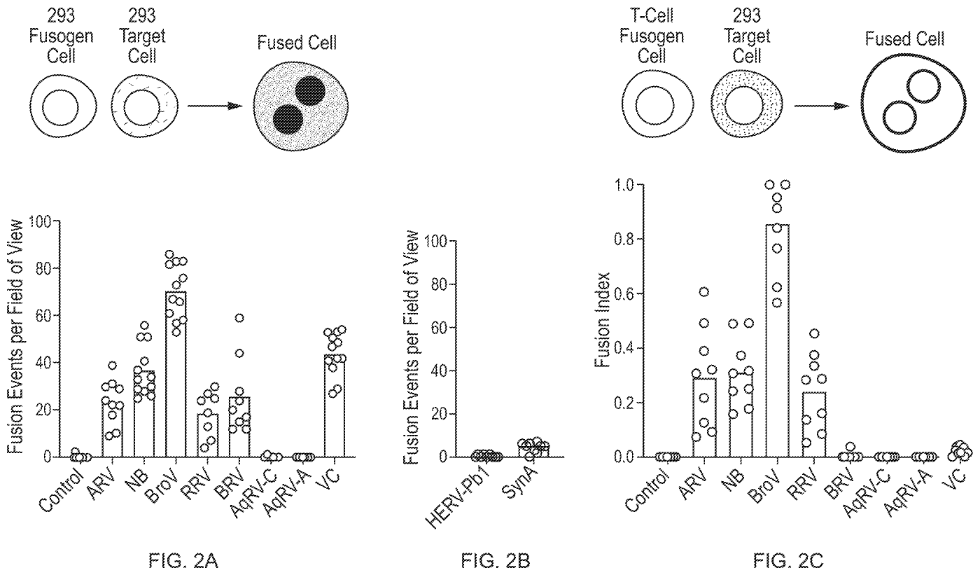

Members of the family of FAST proteins (SEQ ID NOs: 1-20 and 152) were explored for their ability to fuse mammalian membranes. A range of fusion event frequencies (fusion indexes) between cells expressing a natural fusogen (e.g., ARV, NB, BroV, or RRV) and target cells were observed (FIGS. 2A, 2C). FAST proteins were more efficient than endogenous mammalian fusogens for fusion of mammalian membranes (FIG. 2B). Furthermore, FAST proteins mediated heterotypic cell-cell fusion between fusogen-expressing T-cells and 293 epithelial target cells (also known as 293T and as HEK293T, American Type Culture Collection (ATCC), Manassas, VA). (FIG. 2C).

### Reoviridae FAST Fusogens Mediated Fusion in Mammalian Cells

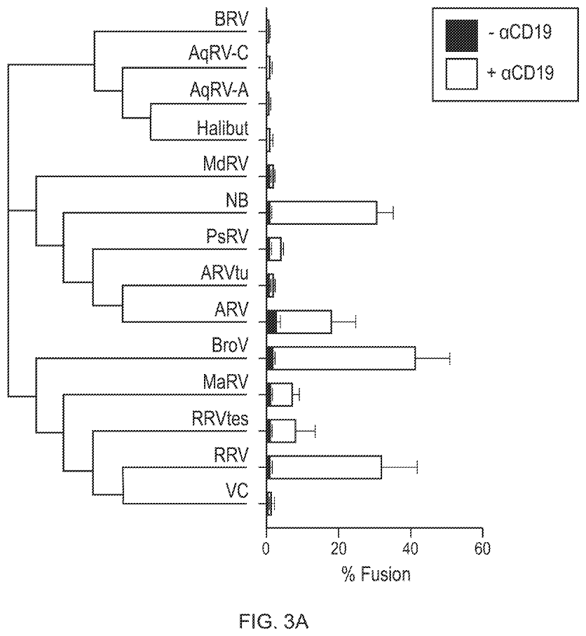

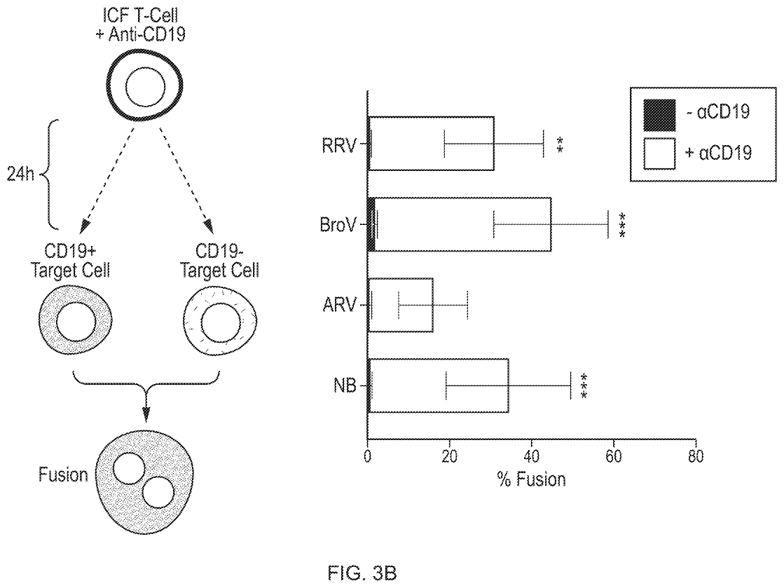

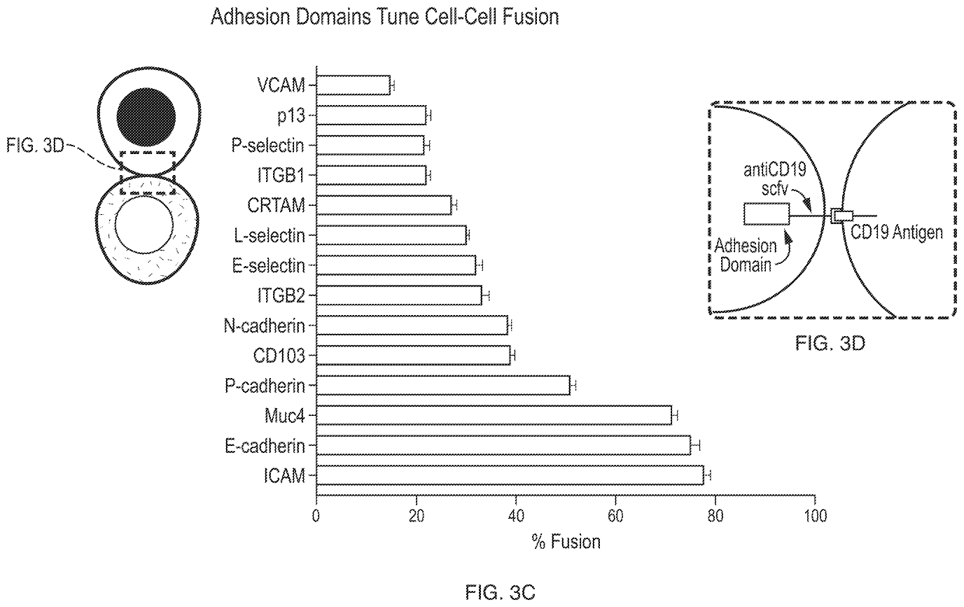

Fourteen FAST fusogen proteins derived from different viruses in the Reoviridae family (ARV, NB, BroV, RRV, BRV, AqRV-C, AqRV-A, Halibut, MaRV, MdRV, RRVtes, PsRV, ARVtu, and VC; SEQ ID NOs: 1-13 and 152, respectively) were expressed in human Jurkat T-cells (ICF T-cells) that either expressed or did not express an anti-CD19 single chain variable fragment antibody (scFv). To determine whether the various FAST fusogens were able to induce fusion with CD19⁺ or CD19⁻ target cells, FAST fusogen-expressing Jurkat cells were co-cultured with Jurkat CD19⁺ target cells for 24 hours at a 4:1 FAST fusogen:target cell ratio, and percent fusion of ICF T-cells with target cells was assessed. Nine of the fourteen FAST fusogen proteins (NB, PsRV, ARVtu, ARV, BroV, MaRV, RRVtes, RRV, and VC) mediated fusion in mammalian cells (FIG. 3A). Furthermore, ICF T-cells expressing certain FAST fusogens (including NB, PsRV, ARV, BroV, MaRV, RRVtes, and RRV) exhibited higher fusion rates when an anti-CD19 scFv was co-expressed (CD19⁺), compared to when no anti-CD19 scFv was co-expressed (CD19⁻) (FIG. 3A). These data demonstrate that several FAST fusogens facilitate fusion between mammalian cells in an antigen-dependent manner.

### FAST Fusogens Exhibited High On-Target Antigen-Dependent Fusion

Four FAST fusogens with demonstrated fusogenic abilities (RRV, BroV, ARV, NB) were expressed in human Jurkat T-cells (ICF T-cells)co-expressing anti-CD19 scFv. At a 4:1 FAST fusogen:target cell ratio, percent fusion of ICF T-cells with target cells was assessed in a 24 hour co-culture assay with CD19⁺ (on-target fusion) or CD19⁻ (off-target fusion) Jurkat target cells. The data demonstrate that FAST fusogens exhibited high on-target fusion and limited off-target fusion via antigen-dependent interactions between the human T-cell and target cell (FIG. 3B). Statistics: Two-Way ANOVA (*: p<0.0332, **: p<0.0021, ***: p<0.0002).

### Adhesion Domains Tuned Cell-Cell Fusion

FAST fusogens are dependent upon cell-cell interactions mediated by both intercellular adhesion and intracellular cytoskeletal signaling. To augment fusion between human ICF T-cells and human CD19⁺ B-cells in an antigen-dependent manner, thirteen intracellular domains of human adhesion proteins (VCAM, P-selectin, L-selectin, CRTAM, ITGB2, ITGB1, E-selectin, P-cadherin, CD103, E-cadherin, N-cadherin, Muc4, and ICAM; SEQ ID NOs:54-66, respectively) were attached to the C-terminus of an anti-CD19 scFv expressed in p13-expressing human Jurkat T-cells (ICF T-cells) (FIG. 3C). ICF T-cells and human CD19⁺ NALM6 (ATCC) cells were co-cultured at a 4:1 ICF T-cell: NALM6 ratio for 24 hours, and percent fusion of ICF T-cells with NALM6 cells was assessed. A range of fusion rates were observed across the thirteen intracellular domains (FIG. 3C). These data demonstrate that fusion rates can be tuned (i.e., that fusion rates are tunable) in p13-expressing ICF T-cells containing an intracellular domain.

### Example 2. Fusogen Structure, Function and Regulation

FAST fusogens are a unique family of fusogens which promote cell-cell fusion through interfacing with host cell cytoskeleton and signaling pathways. To gain a mechanistic understanding of T-cell fusogen regulation, endogenous regulators of fusion were investigated and an engineered library of recombinant FAST fusogens (e.g., engineered fusogens comprising domains from multiple fusogens) was developed.

### Actin and Microtubule Polymerizations Promoted T-Cell Fusion

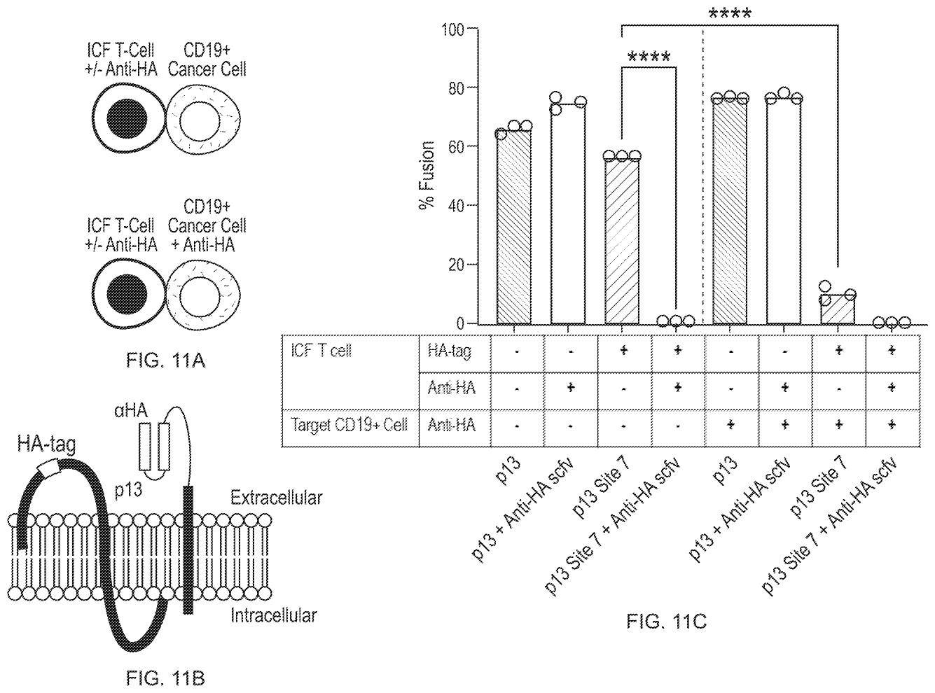

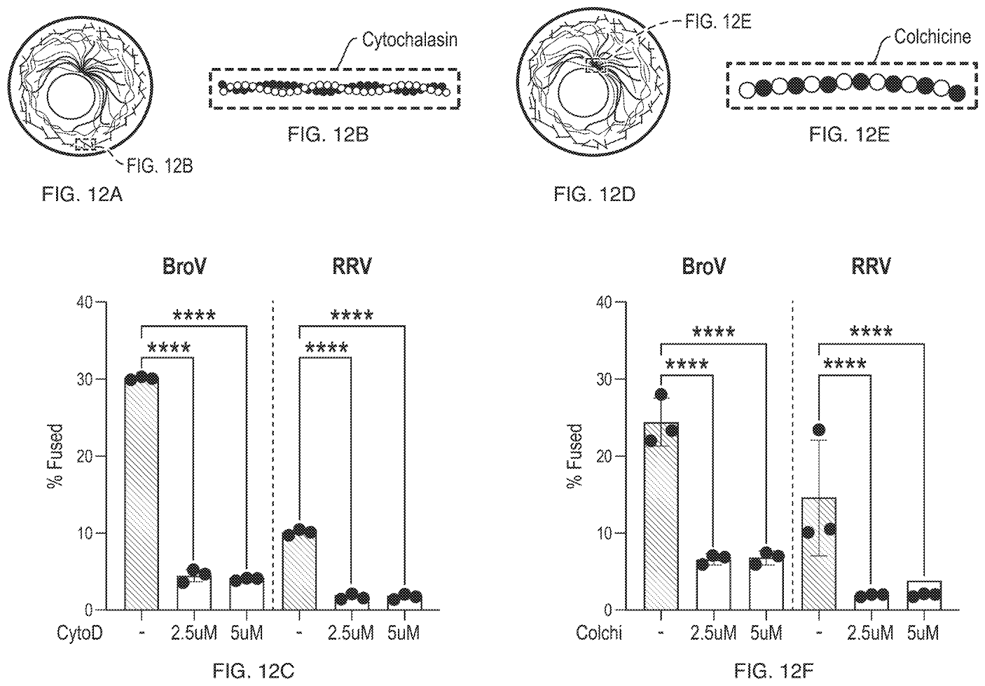

To determine if FAST fusogen-mediated fusion (e.g., ICF T-cell-cancer cell fusion) is mediated via actin cytoskeleton³⁹ and/or microtubule regulation, actin polymerization was inhibited via Cytochalasin D (CAS No. 22144-77-0) (FIGS. 12A-C), and microtubule polymerization was inhibited via Colchicine (IUPAC name: N-[(7S)-1,2,3,10-Tetramethoxy-9-oxo-5,6,7,9-tetrahydrobenzo[a]heptalene-7-yl]acetamide; CAS No. 64-86-8) (FIGS. 12D-F). A significant decrease in fusion was observed in each experiment, suggesting that both actin and microtubule polymerizations promote FAST fusogen-mediated fusion (e.g., T-cell-cancer cell fusion). These results show that exogenous FAST fusogens can co-opt endogenous cellular machinery.

### P13-Mediated Cell-Cell Fusion Showed Dependence on Five Signaling Mechanisms

FAST fusogens co-opt intracellular signaling within mammalian cells to mediate cell-cell fusion. To determine endogenous mechanisms by which the p13 FAST fusogen initiates cell-cell fusion, actin, dynamin, microtubule, PtdIns(4,5)P2 (phosphatidylinositol 4,5-bisphosphate or PIP2; IUPAC name: 1,2-Diacyl-sn-glycero-3-phospho-(1-D-myo-inositol 4,5-bisphosphate); CAS No. 245126-95-8), or interferon-induced transmembrane signaling was targeted using an assay where anti-CD19 scFv/p13-expressing human Jurkat T-cells and CD19⁺ Jurkat target cells were co-cultured at a 1:1 ratio for 24 hours. Inhibiting actin via cytochalasin D or expression of Yersinia outer protein E (YopE) resulted in a significant reduction in cell-cell fusion (FIG. 4, “1”). Inhibiting dynamin, through chemical inhibitor dynasore (CAS No. 304448-55-3), chemical inhibitor MiTMAB (Myr istyl tri methyl ammonium Bromide; CAS No. CAS 1119-97-7), or expression of dominant negative dynamin S61D resulted in a significant reduction in cell-cell fusion (FIG. 4, “2”). Disrupting microtubules via chemical inhibitor colchicine resulted in a significant reduction in cell-cell fusion (FIG. 4, “3”). Inhibiting PtdIns(4,5)P2 via expression of phospholipase C γ (PLCγ) pleckstrin homology (PH) domain resulted in a significant reduction in cell-cell fusion (FIG. 4, “4”). Expressing interferon-induced transmembrane genes IFITM2 and IFITM3 resulted in a significant reduction in cell-cell fusion (FIG. 4, “5”). Taken together, p13-mediated cell-cell fusion was found to be dependent upon these five signaling mechanisms. Statistics: actin/microtubule/dynamin/PtdIns(4,5)P2/IFITM: Unpaired t-test (*: p<0.0332, **: p<0.0021, ***: p<0.0002).

### Engineered Domain Architecture of FAST Fusogens

The architecture of FAST fusogens was explored through a combinatorial domain library. Due to the structural similarity of FAST fusogens, domains amongst FAST fusogens were swapped to generate engineered fusogens comprising domains derived from multiple natural fusogens. These fusogens were engineered to facilitate identification of domains which promote or inhibit fusion. See SEQ ID NOs:21-53.

### Alanine Scan Identified Critical and Fusion-Boosting Regions of P13

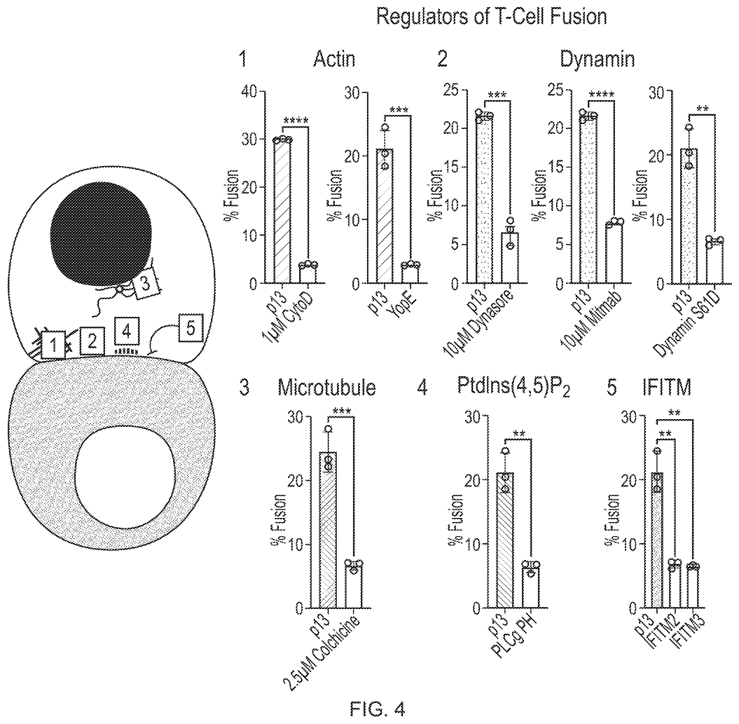

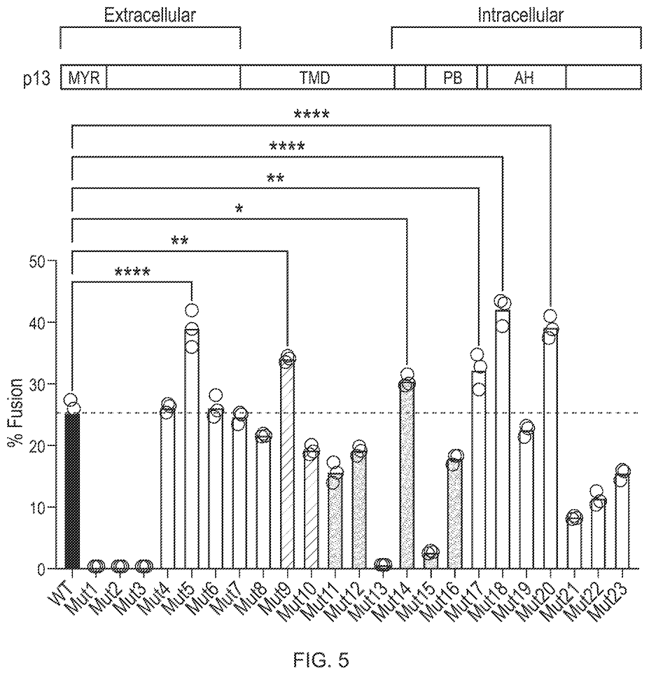

The function of p13 has not previously been characterized at the amino acid level. To determine which amino acids and domains are critical for fusion, an alanine scan of p13 was performed. The p13 protein was mutated with sequential stretches of 4 alanine residues, beginning at amino acid #1 and proceeding to amino acid #113, excluding the transmembrane domain, for a total of 23 unique proteins (SEQ ID NOs:67-111 and 151). Three mutations ablated fusion: (1) Mut 1, comprising mutations within the consensus myristoylation site (MYR: amino acids #1-#2); and (2) Mut 2 and Mut 3, comprising mutations in the critical N-terminus of p13 (FIG. 5). Six mutants, Mut 5, 9, 14, 17, 18, and 20, showed augmented fusion compared to p13 (WT) when expressed in CD19-targeting human T-cells targeting CD19⁺ human B-cells (FIG. 5). Two of these mutants comprise mutations in domains important for p13 function, the polybasic (PB) domain and the amphipathic helix (AH) (FIG. 5). Taken together, these data identified regions of p13 necessary for fusion, as well as alanine mutations capable of promoting fusion (e.g., between human T-cells and human B-cells). Ordinary one-way ANOVA (*: p<0.0332, **: p<0.0021, ***: p<0.0002, ****: p<0.0001).

### Alanine Combination Mutants Showed Augmented Fusion Compared to P13 (WT)

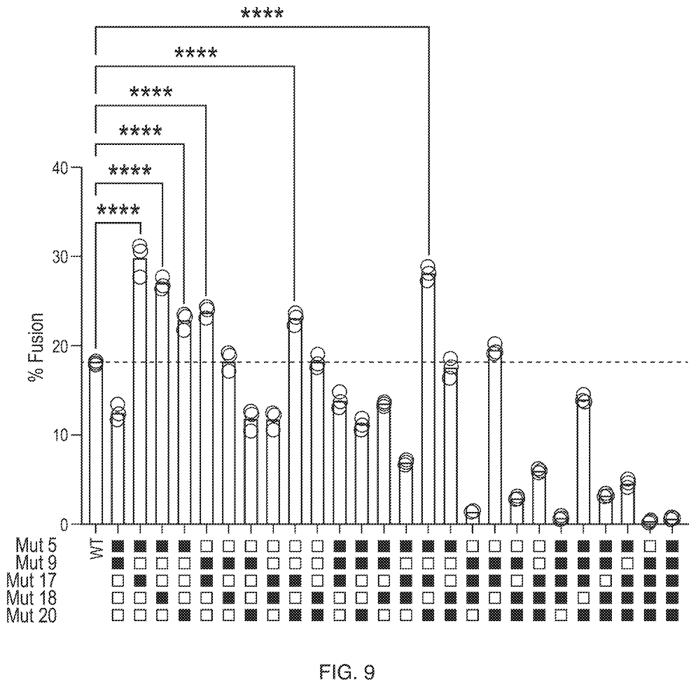

To further define beneficial mutations, the structure/function of p13 was analyzed by performing a combinatorial screen of Mut 5, Mut 9, Mut 17, Mut 18, and Mut 20. Combinations of two, three, four, or all five of the five mutations, which individually displayed increased fusion, were tested for augmentation of T-cell-B-cell fusion. To test these mutants in human T-cells, stably expressing, Doxycycline (IUPAC name: (4S,4aR,5S,5aR,6R,12aS)-4-(Dimethylamino)-3,5,10, 12, 12a-pentahydroxy-6-methyl-1,11-dioxo-1,4,4a,5,5a,6,11, 12a-octahydrotetracene-2-carboxamide; CAS No. 564-25-0) inducible double, triple, quadruple, and quintuple alanine combination mutants (SEQ ID NOs:112-137) were incubated with human B-cell fusion reporter cells at a ratio of 1:1 for 12 hours to assess fusion. Six combinations, each comprising two or three mutations (Mut 5/17, Mut 5/18, Mut 5/20, Mut 9/17, Mut17/20, Mut5/17/20) showed significant augmented fusion compared to p13 (WT) (FIG. 9).

### Example 3. T-Cell Immune Synapse Repurposed Via Synthetic Receptors and Circuits

T-cell engineering efforts have improved the specificity and response of CAR-T therapies. Synthetic Notch (synNotch), a class of synthetic receptors, are antigen-dependent receptors capable of initiating user-defined genetic circuits upon activation¹⁴⁻¹⁵. Recently, these receptors and transcriptional circuitry have been refined for use in the clinic by humanization¹⁶. In line with this research, a new suite of clinically viable genetic switches has been developed which have demonstrated success in controlling multiple genetic programs in primary T-cells using FDA-approved drugs⁹. These tools were harnessed in the development of clinically-focused, engineered immune cell fusion (ICF) T-cells, as illustrated in this example.

During development and disease, programmed cell death is essential for sculpting developing tissues, as well as disposing of aged or damaged cells¹⁷. While the majority of programmed cell death during development is executed through apoptosis, several other forms of inflammatory programmed cell death are implicated in disease, such as necroptosis and pyroptosis¹⁸. These inflammatory forms of programmed cell death initiate adaptive immune responses and promote anti-tumor immunity. Specifically, it has been demonstrated in vivo that activation of necroptosis and pyroptosis within the tumor microenvironment results in cancer regression¹⁹⁻²⁰. However, uncontrolled inflammation, as a result of inflammatory cell death, has been shown to promote tumor metastasis and resistance²¹⁻²⁴.

The immune synapse is an orchestrated interaction between T-cells and target cells, whereby T-cells form specific interactions with target cells through TCR-MHC complexes²⁵. T-cells are capable of killing tumor cells in a regulated and specific fashion, and T-cell therapies have demonstrated sustained success in the clinic²⁶⁻²⁷. While beneficial, T-cells are often ineffective in killing their intended target due to alteration of T-cell signaling or tumor immune escape²⁸.

As described herein, the T-cell immune synapse was repurposed towards targeted fusion with tumor cells for on-demand killing. Furthermore, fully humanized versions of synthetic receptors have been developed, which align ICF towards clinical use¹⁶.

### Cell Death Induced in T-Cells Via Drug Inducible Gene Circuits

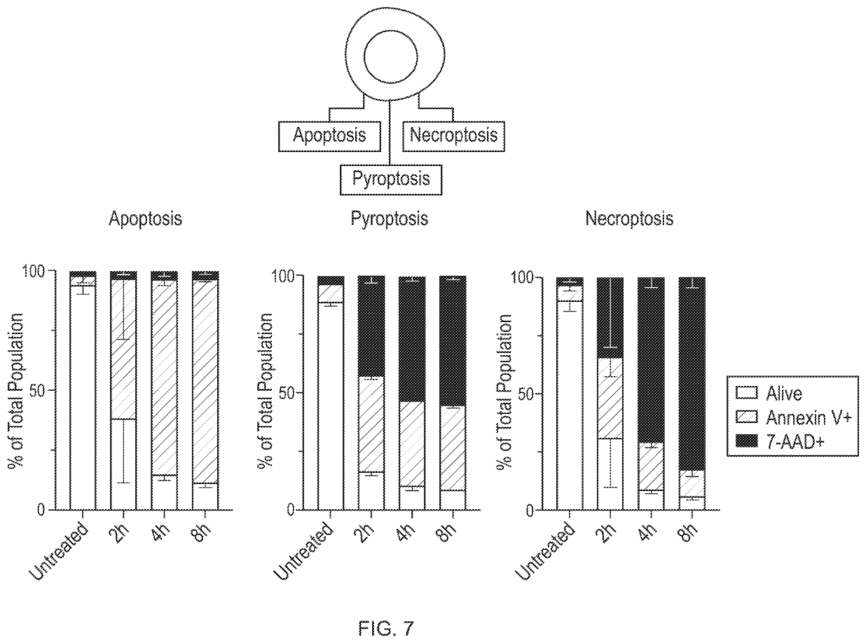

To explore the efficacy of targeted immunogenic cell death, a suite of drug inducible gene circuits, which initiate apoptosis, necroptosis and pyroptosis at different levels within the signaling cascades, was built (FIG. 7). Jurkat T-cell viability was determined via Annexin V (phosphatidylserine, non-lytic death) and 7-AAD (membrane permeability, lytic death) staining at time points up to 8 hours after circuit activation (FIG. 7). The results show that apoptotic pathways led to primarily non-lytic cell death, necroptotic pathways led to increased lytic cell death, and pyroptotic pathways led to more balanced lytic vs. non-lytic cell death.

### Syn Notch Antigen-Specific Circuit Depended on Antibody-Mediated Recognition of Target Cells

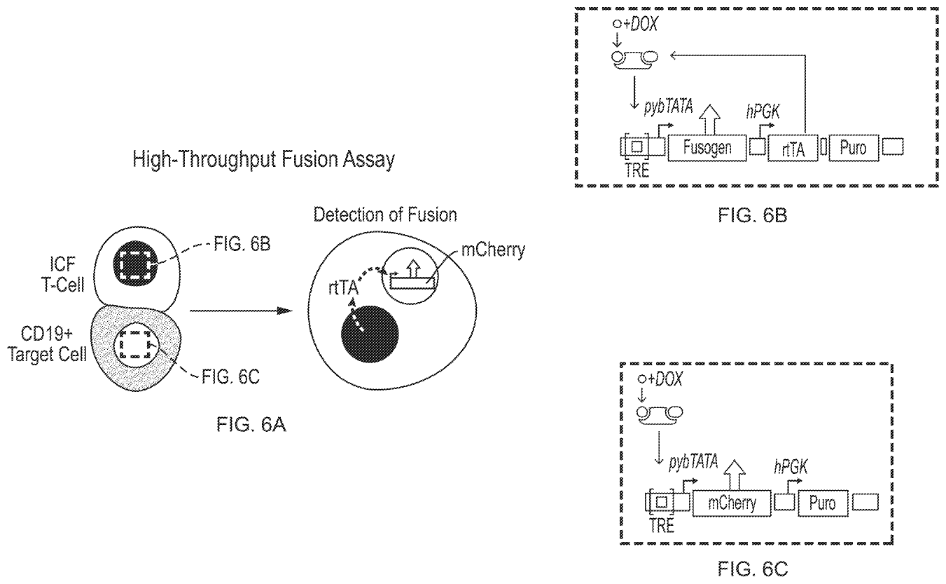

FIGS. 6A-C show a non-limiting example of fusion assays, which is optionally implemented as a high-throughput fusion assay. In one implementation of the assay, stably transduced ICF T-cells were co-cultured with CD19⁺ target cells (either Jurkat T-cells expressing CD19 or CD19⁺ NALM cells) at ICF T-cell:target cell ratios of 0.5:1, 1:1, 2:1, or 4:1 in RPMI (Roswell Park Memorial Institute) 1640 medium with 1 μg/mL of Doxycycline. Doxycycline induced expression of both the fusogen (FIG. 6B) and activated the fluorescent reporter (mCherry; FIG. 6C) upon fusion between ICF T-cells and CD19⁺ target cells. The cells were co-cultured in 96-well plates in a 37° C. incubator with 5% CO₂. After culturing for 24 hours, the cells were collected from the wells and analyzed via flow cytometry to detect fusion events. Upon fusion between ICF T-cell and CD19⁺ target cell, the rtTA transcription factor in the ICF T-cell was transferred to the nucleus of the CD19⁺ target cell. Upon binding of the rtTA transcription factor to the Tet Response Element (TRE), the fluorescent protein mCherry was produced, marking fused cells (illustrated in FIG. 6A, data not shown). Cells were assayed for fusion via both imaging and flow cytometry.

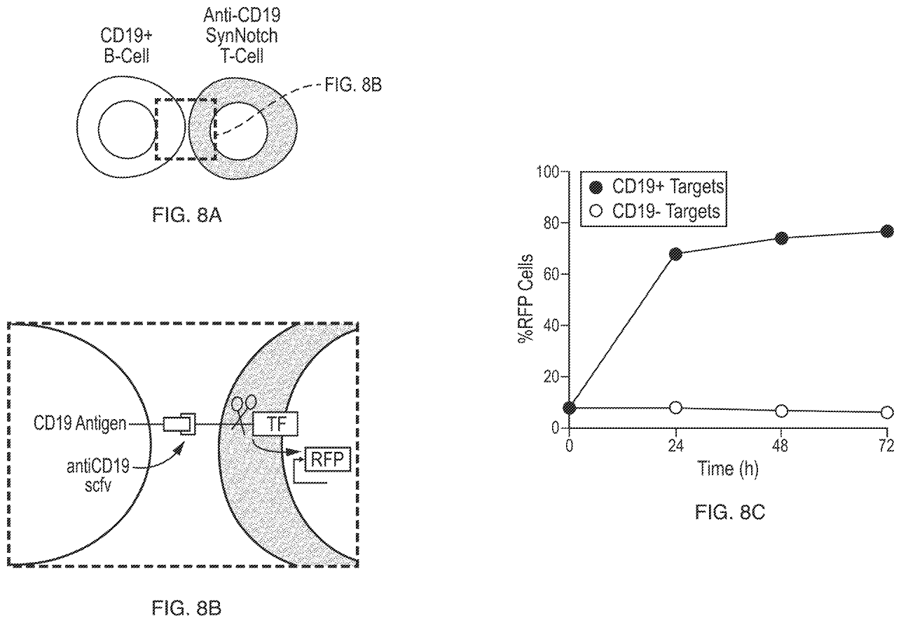

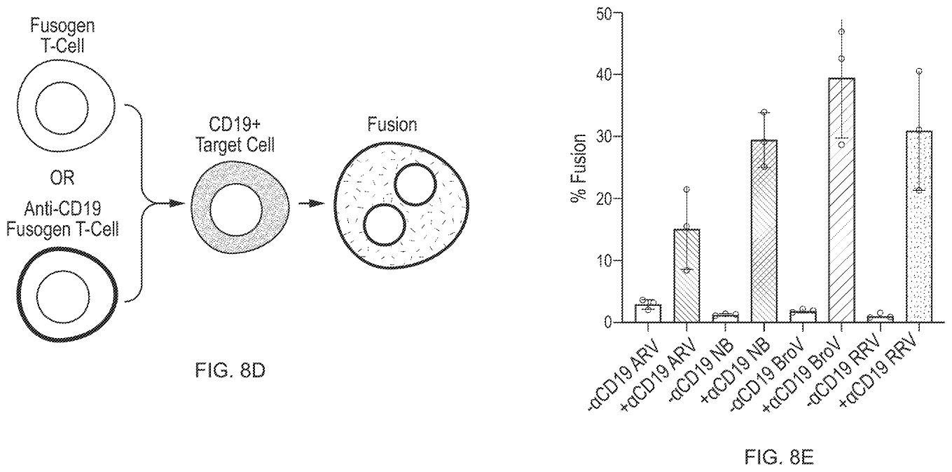

ICF T-cells require antigen-specific synthetic circuit activation to achieve target specific fusion and on-demand cell death switches to eliminate fused cells. To test SynNotch antigen-specific circuit activation, human T-cells were engineered to express a fluorescent protein upon exposure to B-cell tumor antigen CD19 (FIGS. 8A-8B). Within 24 hours, antigen-dependent circuit activation was observed (FIG. 8B). To determine whether T-cell ICF is capable of targeted fusion with human B-cells, ICF T-cells+/−anti-CD19 scfv were co-cultured with human CD19⁺ target cells and monitored for fusion events (FIG. 8C). Fusion was dependent upon antibody-mediated recognition of target cells, as fusion was detected between CD19⁺ cells and anti-CD19 antibody-expressing ICF T-cells, and not between CD19⁺ cells and ICF T-cells lacking anti-CD19 antibody (FIGS. 8D-8E).

Anti-HA scFv Inhibited P13-HA Fusion in Cis and Trans

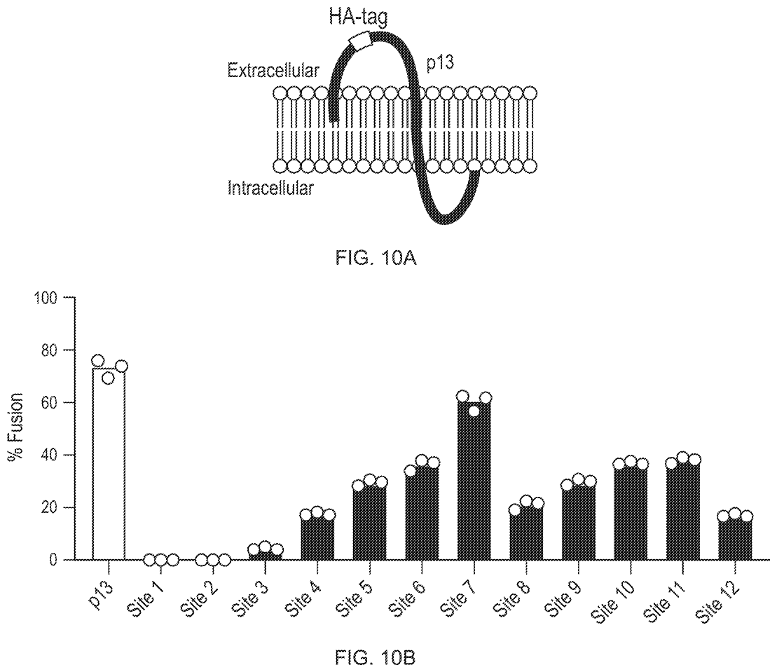

Control over immune cell fusion is important for directing targeted fusion between intended cells and limiting off-target fusion. Inserting a small epitope tag into the extracellular loop of p13 (FIG. 10A) enables stopping fusion upon expression of an antibody against the epitope tag.

To determine where an epitope tag could be inserted within the extracellular loop of p13, 12 mutants, each with an HA epitope tag inserted at a different one of 12 sequential positions in the extracellular loop previously found to be permissible to mutation, were scanned (SEQ ID NOs: 138-149). The majority of the mutants displayed fusion activity, with one mutant (Site 7, SEQ ID NO: 144), approaching the level of p13 (FIG. 10B).

Fusion was inhibited when ICF-T cells expressed HA-tagged p13 (HA-p13, p13 Site 7) and target cells expressed anti-HA scFv51 (SEQ ID NO:150) (FIG. 11A, 11C). Fusion was similarly inhibited when ICF-T cells co-expressed HA-p13 and anti-HA scFv (FIG. 11B), whether or not target cells also expressed anti-HA scFv (FIG. 11A, 11C).

These data suggest that p13-mediated fusion (e.g., between T-cells and target cells) can be controlled at the protein level through use of an antibody or antigen-binding fragment thereof.

### Example 4. (Prophetic) Developing Precision T-Cell Fusion for Liquid and Solid Tumor Ablation

Example 4 is a prophetic example describing future research. Current CAR-T therapies exhibit off-target killing, ineffective killing or over-activation⁴. These issues underscore the need for a more efficient, targeted cell therapy. Gaining control over fusogen activation is tantamount to ensure targeted cell fusion. FAST fusogens exhibit dependence upon endogenous cell signaling pathways, such as actin and calcium signaling²⁹. It is important to determine the molecular players which mediate fusion.

To induce specific tumor ablation using user-defined control over both the timing and mode of cell death, fusion is controlled in an antigen-specific manner using a suite of engineered synthetic receptors (e.g., SynNotch; as disclosed above, the SynNotch platform functions in immortalized T-cells; see, e.g., FIGS. 8A-8D); specific and targeted fusion between engineered ICF T-cells and tumor cells is engineered using an existing genetic toolbox⁹; and activation of cell death is controlled through the SynZiFTR platform. Function of fusogens in primary T-cells is further explored and the effects of ICF-mediated programmed cell death on tumor cell burden is explored. To align this platform with clinical use, humanized, antigen-activated synthetic circuits are targeted against both liquid and solid tumors, using human B-cell lymphoma and lung tumor models, respectively. In sum, ICF seeks to obviate inefficient killing of target cells, and unlock unprecedented control over targeted tumor eradication.

CRISPR activation and CRISPR inhibition screens in primary T-cells (fusogen-expressing cells) and human B-cell lymphoma cells (target cells) are used to determine the molecular underpinnings of T-cell ICF. Employing a high-throughput fusion assay disclosed herein (see, e.g., FIG. 6), which rapidly detects cell fusion through a fluorescence readout, genetic determinants of fusion are screened for via flow cytometry sorting of fused and un-fused cells. Hits from these screens are validated through over-expression in target cells for their ability to modulate fusion. Together, these efforts detail the mechanism by which fusogens interface with the host cell. These example experiments comprise three areas of research that each advance a novel cell-based therapy for treatment of B-cell lymphoma and lung cancer. For example, T-cell ICF specifically targeting B-cell lymphoma is developed using clinically-viable engineered receptors and gene circuits. Design principles of FAST fusogens and genetic dependencies of cell fusion are uncovered to characterize regulators of ICF and inform future T-cell ICF design. The suite of inducible cell-death switches disclosed herein is used to determine the response of ICF-dependent immunogenic cell death within the B-cell lymphoma and lung cancer tumor microenvironment. If sufficient response to targeted, immunogenic cell death is observed, T-cell ICF can be employed in a clinical trial for treatment of B-cell lymphoma and lung cancer. The scope of ICF can be expanded to other cancers as well as new modalities of tumor ablation.

### Uncovering Functional Mechanisms of Fusion to Improve ICF Design

The FAST family of fusogens have evolved in wide array of hosts, yet many share common structural features and signaling domains³⁰.

Fusion is studied using at least two approaches. For example, in order to better understand and control FAST fusogens, genome-wide CRISPR screens are performed in primary ICF T cells to identify genes and gene networks which inhibit fusion. Using the high-throughput fusion assay disclosed herein, a genome-wide CRISPR screen is performed to identify positive and negative regulators of fusion. The architecture of FAST fusogens is explored through a combinatorial domain library. Due to the structural similarity of FAST fusogens, domains amongst FAST fusogens are swapped to identify domains which promote or inhibit fusion.

To explore the conserved nature of these domains, and their fusogenic potential, a pooled library of fusion domains is designed; and the library and disclosed engineered fusogens (SEQ ID NOs:21-53) are tested. Signaling domains are arranged in combinatorial fashion in a pooled library encompassing all currently identified FAST fusogens. The library of recombinant fusion domains is inserted into primary T-cells and novel arrangements which promote or ablate fusion are identified. Results from this screen will identify the contribution of signaling domains, their modularity, and uncover novel arrangements to uncover functional mechanisms of T-cell-tumor cell fusion, which is used to improve ICF design.

### High-Throughput Design and Evaluating Fusogen Regulatory Domains.

FAST fusogens share structural and domain similarity yet display a range of fusogenic potential. A combinatorial FAST fusogen domain library, whereby domains across the FAST family are shuffled, is constructed to determine combinations of domains which promote or inhibit fusion. Using a DNA-barcoded library, domains which promote T-cell:target fusion are extracted. Furthermore, prevalence of domain-assigned barcodes is analyzed to determine fusogen domain efficiency.

### Genetic Basis of Cell-Cell Fusion

CRISPR activation and inhibition screens are used to identify genes and pathways which regulate cell fusion. T-cells expressing both fusogen and Cas9 components are created for activation and inhibition. The T-cells are transduced with a genome-wide activation or inhibition CRISPR library, followed by co-culturing with cancer target cells expressing the fusion reporter disclosed herein. Gene-associated barcodes are extracted from fused or not fused cells, separated using flow cytometry cell sorting, and negative and positive regulators of cell fusion are identified by bioinformatic analysis. In sum, these activities will establish ICF in primary cells and the efficiency of inducible cell death.

### Exploring User-Defined Immunogenic Cell Death in the Tumor Microenvironment

Programmed cell death is often characterized as either apoptotic or non-apoptotic cell death. Apoptotic cell death actively inhibits immune responses within the tumor, whereas several forms of non-apoptotic cell death elicit an immune response³¹. Non-apoptotic programmed cell death is largely restricted to instances of disease. These types of cell death have immunogenic potential, as pyroptosis and necroptosis are being evaluated for their therapeutic potential in tumors 19-20.32. However, these modes are inaccessible with current T-cell therapies. Furthermore, precise delivery and on-demand activation of ICF within the tumor is a challenge.

A suite of inducible death switches, such as that described in FIG. 7, and which encompasses apoptosis, pyroptosis and/or necroptosis is configured to be inducible at two levels within the signaling cascades, i.e., activation of an upstream initiator and/or downstream executioner of signaling, has been generated. For example, a switch induces expression of proteins RIPK3 for upstream and/or MLKL for downstream signaling in necroptosis.

In order to unlock on-demand tumor killing upon fusion, clinically-inspired gene circuits as described above, which activate cell death upon either ligand binding or FDA-approved small molecule administration, permit the study of precise and tunable immunogenic cell death within the tumor microenvironment in in vitro models and in subjects. These tools, and clinically-focused designs, will be important for dissecting the targeted benefit and response to immunogenic cell death within the tumor microenvironment via ICF.

### Designing and Evaluating Receptors and Genetic Circuits for CD19⁺ B-Cell ICF

A suite of engineered receptors and genetic circuits, which promotes ICF upon interfacing with a tumor cell, is assembled. A CD19⁺ specific synthetic receptor, which will activate ICF machinery in an antigen-dependent manner, is constructed. This circuit is tested in vitro, in primary T-cells, for fusion to CD19⁺ human B-cells. Kinetics and efficacy of fusion is determined via a high-throughput fusion reporter assay disclosed herein. Receptor design described herein is ported to humanized receptors to advance ICF towards use in a clinical setting. These receptors are tested for fusion kinetics and efficiency.

### Evaluating Inducible Tumor Cell Ablation Through Cell Death Circuits

To activate cell death programs in fused cells, inducible cell death programs are designed and tested further to the designs and testing disclosed herein. The circuits comprise initiators and executioners of the apoptotic cell death pathway. Kinetics of cell death upon circuit activation are measured using flow cytometry, and specificity of pathway activation is determined through use of chemical cell death inhibitors. These designs are incorporated into clinically viable genetic circuits using the SynZiFTR platform. These circuits are tested for kinetics and paired with ICF circuits to create ‘fuse and kill’ T-cells. Fuse and kill T-cells are tested both in vitro and in vivo for their ability to target and eliminate CD19⁺ B-cells. In vitro fusion and cell death is measured via flow cytometry. In vivo experiments are monitored at several time points for target fusion and death via whole mouse luciferase imaging. Optimal dosage of ICF and induction of cell death circuits are determined via longitudinal studies to determine fusion and cell death kinetics in vivo.

### Designing and Evaluating Genetic Circuits for Solid Tumor ICF

ICF targeted towards solid tumors is under development. To test ICF in a solid tumor setting, engineered receptors and circuits are created and tested for their ability to promote T-cell and lung cancer fusion in vitro. ICF T-cells activated through lung cancer specific antigen ALPPL2, MUC1, and/or LunX are designed using antigen-activated synthetic receptors³³⁻³⁶. These circuits are tested for their ability to fuse with SK-OV-3 lung cell lines using the high-throughput fusion reporter assay disclosed herein. Third, lung cancer susceptibility to apoptosis, pyroptosis, and necroptosis are tested using the inducible cell death circuits disclosed herein (see, e.g., FIG. 7). To determine whether ICF is able to ablate solid tumors in vivo, the A549 lung cancer solid tumor model is employed, ‘fuse and kill’ T-cells are administered, and tumor size and spread are monitored via whole mouse luciferase imaging. These results will establish ICF for solid tumor targeting, elucidate lung cancer response to inducible cell death and inform future ICF design and application.

This platform is being expanded to target other cancers such as breast cancer and glioblastoma, and immunostimulatory programs such as panoptosis or STING activation³⁷⁻³⁸. ICF can be translated to the clinical setting to treat patients with liquid and/or solid tumors where current cell-based therapies are ineffective.

To promote fusion or cell death in primary cells, circuit designs may be revised towards previously demonstrated circuits in primary cells. In the event where ICF is too potent or lethal in vivo, combinatorial therapy, whereby ICF is combined at varying ratios with CAR-T, is tested to determine safe dosage and efficacy. Furthermore, which cell death modality best suited towards tumor type is determined, as some modalities may have varying efficacy. If excessive fusion is observed within the tumor, fusogens with reduced fusogenic capacity will be used.

### Generating Logic-Based Fusion Circuits

Auto-regulatory fusion circuits which control fusion are designed based on insights from the CRISPR screens. These synthetic circuits are constructed to control the rate of fusion and avoid excessive fusion upon T-cell-target engagement. Genes which inhibit fusion are selected, and whether their overexpression inhibits fusion is tested. Genes which promote fusion are knocked out, and whether this inhibits T-cell-target fusion is tested. Synthetic circuits, which inhibit fusion upon detection of an off-target molecule or used-defined input, are constructed.

To obviate spurious positive-regulator hit from these screens, cell lines lacking genes encoding one or more potential positive-regulator hits are created to assess fusion. To avoid spurious negative-regulator hit from these screens, genes encoding one or more potential negative-regulator hits are overexpressed to assess their impact on fusion. These two approaches clarify regulators of cell fusion. In the event that logic gated fusion is unable to restrain fusion, regulation of the fusogen itself is explored via insertion of degradation domains or proteolytic cleavage sites, which permits control over fusion. These alternative approaches can result in a logic gated fusion circuit.

### Benefits and Responses to Immunogenic Cell Death in the Tumor Microenvironment

Cell death within the tumor microenvironment is essential for tumor regression, however signals from dying tumor cells can decrease immune response, attract immunosuppressive cells, and promote tumor growth⁴⁰⁻⁴². In contrast to immunoquiescent apoptosis, pro-inflammatory cell death such as pyroptosis and necroptosis have been proposed as novel therapeutics to mount an increased adaptive immune response and promote durable tumor regression in both B-cell lymphoma and lung cancer^{20,32,43-45}.

To explore targeted and regulated T-cell-mediated immunogenic cell death, the ICF platform disclosed herein is used to fuse T-cells with tumor cells and initiate pro-inflammatory cell death programs. Using this approach, the response of tumor cells and immune cells to pro-inflammatory cell death is explored and tumor regression is characterized. Furthermore, while canonical cell death pathways is tested and utilized, novel gene dependencies which promote tumor death are identified through CRISPR-mediated screening. Designing and Evaluating Inducible Immunogenic Cell Death Pathway Circuits Using Clinically Inspired Circuits

Activation of pyroptosis and necroptosis is evaluated through inducible expression of cell death signaling cascades using the clinically focused SynZiFTR platform. Because initiators of these pathways often involve protein multimerization, higher-order clustering of initiators are surveyed through use of controllable oligomerization domains. To evaluate efficacy of cell death circuit initiation, flow cytometry and cell viability dyes are used to profile dynamics of cell death upon circuit induction.

### Inducing Targeted Immunogenic Cell Death in B-cell Lymphoma and Lung Cancer

Using ICF, CD19⁺ B-cells are targeted for ablation through inducible pyroptotic and necroptotic cell death. Validated cell death circuits are activated upon fusion of T-cells to CD19⁺ B cells in vitro. The rate of killing is determined via flow cytometry and proinflammatory cytokines are measured via ELISA. To determine whether this paradigm is capable of tumor eradication in vivo, titrated CD19-targeted ICF T-cells are administered to a NALM6 B-cell lymphoma mouse model. The death switch is activated at defined periods; tumor eradication is measured through whole animal luciferase; inflammation in the tumor microenvironment is detected through biopsy, cytokine ELISA, and targeted metabolomics; and adaptive and innate immune response is identified through single cell RNAseq.

Solid tumor lung cancer is also ablated by ICF. Lung cancer is specifically targeted through ALPPL2 antigen, followed by induction of cell death pathways. The targeting and cell induction methods are tested in vitro, via both flow cytometry and imaging measurements of cell depletion. A human A549 xenograft mouse model of lung cancer is used to assess, in vivo, lung-targeted ICF T-cells equipped with immunogenic cell death circuits. Similarly, response to cell death is measured via whole animal luciferase imaging, biopsy ELISA, and single cell RNAseq.

### Eradicating Tumors Through CRISPR Dependency Screen

Cancer cells exhibit unique genetic dependencies to ensure proliferation⁴⁶⁻⁴⁸.

Genetic dependencies in NALM6 B-cell lymphoma and A549 lung cancer cells are explored using death-seq⁴⁹, a high-throughput cell death assay. Hits from these screens are validated through individual gene knockouts and cell viability measurements. ICF cells with an inducible CRISPR circuit are engineered to mediate targeted cancer cell gene disruption upon fusion. ICF-mediated gene dependency cell death are assayed in vitro, and hits are applied to both lymphoma and lung in vivo tumor models. These approaches unlock new modes of targeted cell killing, as well as unveiling response to genetic dependency cell death within the tumor microenvironment.

A goal is to generate inducible pro-inflammatory cell death circuits and to specifically eradicate tumor cells. To avoid ineffective cell death induction in vitro, initiator and effector proteins in the apoptotic, pyroptotic and necroptotic signaling pathway are explored in addition to their multimeric state. To bypass ineffective cell death induction in vivo, optimal dosage of cell death circuit activation is determined through a dose response curve, based upon previous ranges. In the case that circuit activation is ineffective, circuit design is re-evaluated and re-designed to incorporate in vivo validated design. Furthermore, cell death resistant cells are isolated and profiled via RNA-seq to identify resistance mechanisms. These alternative approaches identify robust cell death circuits and permit exploration of targeted immunogenic cell death.

### Using ICF Technology for Immortalization of Human B-Cells

Hybridoma technology permits the fusion of primary B-cells with immortalized cell lines for the generation of monoclonal antibodies; however, the technology is limited by the fusion step, which has an efficiency of 5-10%. ICF technology demonstrated >70% rates of fusion with human B-cells, highlighting the transformative potential of ICF to unlock a bottleneck in hybridoma generation.

To generate hybridomas, i.e., to immortalize B-cells, ICF cells stably expressing one or more natural and/or engineered exogenous fusogens are co-cultured with and fuse with B-cells (e.g., antibody-expressing B cells). The ICF cells are immortalized or functionally immortal mammalian cells, such as immortalized myeloma cells. In some embodiments, ICF cells express one or more B-cell targeting molecules, for example, an anti-CD19 scfv, an anti-CD27 scfv, and/or a different scFv capable of targeting a B-cell surface antigen. Following fusion between ICF cells and B-cells, clones of fused cells are cultured and screened, for example, for production of a desired antibody.

### SEQUENCES

Natural Fusogen Sequences
ARV (ARVch)-p10-Avian orthoreovirus
MLRMPPGSCNGATAVFGNVHCQAAQNTAGGDLQATSSIIAYWPYLAAGGGF
LLIVIIFALLYCCKAKVKADAARSVFHRELVALSSGKHNAMAPPYDV (SEQ ID NO: 1)
NB-Nelson Bay-Nelson Bay orthoreovirus
MSSDCAKIVSVFGSVHCQSSKNSAGGDLQATSVFTTYWPHFAIGCGIIVVILL
LGLFYCCYLKWKTSQVKHTYRRELIALTRSHVHSTPSGISYV (SEQ ID NO: 2)
BroV-p13-Broome orthoreovirus (Broome reovirus)
MGSGPSNFVNKVDGASAPIKEHAIPSLTSDLKDYLYTIVTAVILLVILWFLYRY
YKDKKARKKKEDILLRLYGRGLNLSRLDPSVICSLGGSAPNLQHRGLERTEDKLVNPFI
(SEQ ID NO: 3)
RRV (RR Vlep)-p14-Reptilian orthoreovirus
MGSGPSNFVNHAPGEAIVTGLEKGADKVAGTISHTIWEVIAGLVALLTFLAFG
FWLFKYLQKRRERRRQLTEFQKRYLRNSYRLSEIQRPISQHEYEDPYEPPSRRKPPPPPYS
TYVNIDNVSAI (SEQ ID NO: 4)
BRV-p15-Baboon orthoreovirus
MGQRHSIVQPPAPPPNAFVEIVSSSTGIIIAVGIFAFIFSFLYKLLQWYNRKSKN
KKRKEQIREQIELGLLSYGAGVASLPLLNVIAHNPGSVISATPIYKGPCTGVPNSRLLQITS
GTAEENTRIILNHDGRNPDGSINV (SEQ ID NO: 5)
AqRV-C-p16 Aquareovirus-C
MPCQDTVSLSIQHTSVYVQHSCCVSTTTSASTSATALGLGCLACGIVGVLVV
AGGLCCLINGRCPSCRRLALRSRSWKPPPTSLCTNQPLAFNLRDLTRSNIRCTSDPRSVEL
LSDVHSVVSHRECPPAYDSLDFEPTEYTPEAFQ (SEQ ID NO: 6)
AqRV-A-p22 Aquareovirus-A
MGNTISNTVQYTVLQIDRSCCIKTSLTATSEATSWAIPPLAICCCCCICCTGGL
YLVHSGRFPGLSRRLDVLGGSGSTPKHSLRSHRHPKPRVHRVSFSDSSDSSDISDLELPRH
GSHPLAHSFRPEVDRHRPRPSTQVQQTSFIPLVPLRSGSSLDDGIVRSQPSRDSRPHEQFE
DWLQQAHLLRPGRVSGSTNPFT (SEQ ID NO: 7)
Halibut-Atlantic halibut reovirus
MPCGDTVSTTVQYTVLQIDRSCCISTSLTATSEATSWALPPLCICCCCLVCTGL
GIYAIQRGHCPGISRRLDVLRSSGSSSKQPVCDQRRERSRIYRPRSSDSFGFDDQSTLDLH
RDSPRVLENQFRLENNRSRSSPKPLSVQPSAFDESIRLHIQSLIRTQPQHDDSNRNQDSLIH
EHIPRRGNPFI (SEQ ID NO: 8)
MaRV-Mahlapitisi orthoreovirus
MGSGPSNFVNHASGEAIISGLSDQTNRLGSLLSQNVYNIIYFFVIGGLILSAGY
GLYKYCKYKQRRAKNLTRQLSRELIDLNRKIEHISGGKIPATKPSAPRYTPPCYKEPIYNE
VCEGGFYGNC (SEQ ID NO: 9)
MdRV-Muscovy duck orthoreovirus
MADGACNHATSIFGAVYCQISQNIAHGNIDSYTSWTSYLPPILGGGFGLIVLL
VLVVLIVYCCKHSKILSAVKATDSAVTTLLRDVAPANPDPVQVV (SEQ ID NO: 10)
RRVtes-Reptilian Testudine orthoreovirus
MGNGPSNFVNHSPAEAILTGLDKNTHSITSTFTNGIKELLVGLLVLILFFVAAG
AACWYWRKRRAKRRRLTKYQIRFLNDFSRSARLNQTPIPVASKRETWPQKTRSVSPPPY
TANYVNVNDYDEAESAPTFRRY (SEQ ID NO: 11)
PsRV-Piscine Orthoreovirus
MPRLPGGSCNGATSVFGSVHCQAAQNTAGGDLQATSSLVSYWPYLAAGGSL
LLIVVLVALFFCCRAKIKADATKNVFRRELIALTTKSGHDRPPSYEV (SEQ ID NO: 12)
ARVtu-Avian orthoreovirus turkey
MSRPSSGSCNGALAVFGNVHCQAAQNSAGGDLQATSSLIAYWPYLAAGGGV
LLLLIVVVAVIYCKAKVKADAARNVFFRELVALNEVKCNAVPPSYRV (SEQ ID NO: 13)
Feline Rotavirus Species I NSP-1 (FeRVI):
MGGRISQQLSQQNTYHIASGNSQIYSQDQKTNQQVAVAFEVCHLSILFIIALALVVHVTC
SARKRGCAVKSFEKPFLTNV (SEQ ID NO: 14)
Canine Rotavirus Species I NSP-1 (CaRVI):
MGNTYNVHNNQQVSSNTVHGSGQIHSEDQKTSSQITTIVQFSNLSLLFLIALFL
FISLLFKCDKNRKKQKWNTREIIEL (SEQ ID NO: 15)
Gallinaceous Rotavirus Species G NSP-1 (GaRVG):
KLNLQDQKTTSLESTQLLLGIGAIVVVALIILLIFSLILNCYLCSKLKRKNGYLK
RERKISNCRDKGLDKLILSKSDDIASSCV (SEQ ID NO: 16)
Avian Rotavirus Species G NSP-1 (AvRVG)
MGSHQSSYQVNNONTIISNSKNLDFKPSTSSSLSQVNLFFVIGAAVGVFLLTV
LIISIILNIYLCRRLKNGRKHDGRTSFQSGTTSRHNAKMDEREHLQSNTNV (SEQ ID
NO: 17)
Caprine Rotavirus Species B NSP-1 (CaRVB)
MGSSQSSLQSQVHSTNIHSQHSSIHLQGTSTANFTTQHIILTVGAALIALLLTSL
IFSCICNCYLYSKLRNGFQTVSQHVRRKERSHTNIPGQQIRPDMYV (SEQ ID NO: 18)
Porcine Rotavirus Species B NSP-1 (PoRVB)
MGNRQSSLQSQTHRTDINSHNSNIYLQSASSAEFRTQHILVVAGAALIALLFA
FLVSSLVCNCYLLRRLRNGPRKIYRTGKIQEGSYSSLSKQFIRPDHFV (SEQ ID NO: 19)
Human Rotavirus Species B NSP-1 (HuRVB)
MGNRQSSAQLNSHLTHINSQNSNLFISDSKTAVFHTQHILLAAGVGIIATLLVL
LLCSCVLNCYLCRKLKRTNGVSSLLERNIRQNGSSAKIYVKPVMQSSTIIEEA (SEQ ID
NO: 20)
Veiled Chameleon (VC) (PVC)
MGSGPSNFVNHAPGEAIVTGLEKGADKVAGTISHTIWEVIAGLVALLIFLAIGF
WLFKHLQKRRERARQLSEFQKRYLRNSYRLSEVQRPISLHEYEDPYEPPSRKKAPPPPYS
TYVNIDDVSAV (SEQ ID NO: 152)
Engineered Fusogen Sequences
P15-PsRV-RRV
MGQRHSIVQPPAPPPNAFVESGYWPYLAAGGSLLLIVVLVALFFCCGSKHLQ
KRRERARQLSEFQKRYLRNSYRLSEVQRPISLHEYEDPYEPPSRKKAPPPPYSTYVNIDD
VSAV (SEQ ID NO: 21)
P13-PsRV-MdRV
MGSGPSNFVNKVDGASAPIKEHAIPSLTSDLKDSGYWPYLAAGGSLLLIVVL
VALFFCCGSKHSKILSAVKATDSAVTTLLRDVAPANPDPVQVV (SEQ ID NO: 22)
P10-MdRV-NB
MLRMPPGSCNGATAVFGNVHCQAAQNTAGGDLQATSSIIASGVLGGGFGLIV
LLVPVVLIVYCCGSLKWKTSQVKHTYRRELIALTRSHVHSTPSGISYV (SEQ ID NO: 23)
P13-MdRV-MARV
MGSGPSNFVNKVDGASAPIKEHAIPSLTSDLKDSGILGGGFGLIVLLVLVVLIV
YCCGSKYCKYKORRAKNLTRQLSRELIDLNRKIEHISGGKIPATKPSAPRYTPPCYKEPIY
NEVCEGGFYGNC (SEQ ID NO: 24)
P10-MdRV-NB
MLRMPPGSCNGATAVFGNVHCQAAQNTAGGDLQATSSIIASGILGGGFGLIV
LLVLVVLIVYCCGSLKWKTSQVKHTYRRELIALTRSHVHSTPSGISYV (SEQ ID NO: 25)
P22-P10-MdRV
MGNTISNTVQYTVLQIDRSCCIKTSLTATSEATSSGYWPYLAAGGGFLLIVIIF
ALLYCCGSKHSKILSAVKATDSAVTTLLRDVAPANPDPVQVV (SEQ ID NO: 26)
P10-PsRV-ARVch (ARV)
MLRMPPGSCNGATAVFGNVHCQAAQNTAGGDLQATSSIIASGYWPYLAAGG
SLLLIVVLVALFFCCGSKAKVKADAARSVFHRELVALSSGKHNAMAPPYDV (SEQ ID
NO: 27)
Halibut-RRVtes-ARVch (ARV)
MPCGDTVSTTVQYTVLQIDRSCCISTSLTATSEATSSGLLVGLLVLILFFVAAG
AACWYWGSKAKVKADAARSVFHRELVALSSGKHNAMAPPYDV (SEQ ID NO: 28)
MARV-pNB-p10
MGSGPSNFVNHASGEAIISGLSDQTNRLGSLLSQNSGYWPHFAIGCGIIVVILL
LGLFYCCYGSKAKVKADAARSVFHRELVALSSGKHNAMAPPYDV (SEQ ID NO: 29)
p13-p13-p10
MGSGPSNFVNKVDGASAPIKEHAIPSLTSDLKDSGYLYTIVTAVILLVILWFLY
RYYGSKAKVKADAARSVFHRELVALSSGKHNAMAPPYDV (SEQ ID NO: 30)
p13-p13-PsRV
MGSGPSNFVNKVDGASAPIKEHAIPSLTSDLKDSGYLYTIVTAVILLVILWFLY
RYYGSRAKIKADATKNVFRRELIALTTKSGHDRPPSYEV (SEQ ID NO: 31)
p13-pNB-p10
MGSGPSNFVNKVDGASAPIKEHAIPSLTSDLKDSGYWPHFAIGCGIIVVILLLG
LFYCCYGSKAKVKADAARSVFHRELVALSSGKHNAMAPPYDV (SEQ ID NO: 32)
p13-pNB-PsRV
MGSGPSNFVNKVDGASAPIKEHAIPSLTSDLKDSGYWPHFAIGCGIIVVILLLG
LFYCCYGSRAKIKADATKNVFRRELIALTTKSGHDRPPSYEV (SEQ ID NO: 33)
p13-pVC (VC)-pNB
MGSGPSNFVNKVDGASAPIKEHAIPSLTSDLKDSGIWEVIAGLVALLIFLAIGF
WLFGSLKWKTSQVKHTYRRELIALTRSHVHSTPSGISYV (SEQ ID NO: 34)
p13-RRVlep (RRV)-MdRV
MGSGPSNFVNKVDGASAPIKEHAIPSLTSDLKDSGIWEVIAGLVALLTFLAFG
FWLFGSKHSKILSAVKATDSAVTTLLRDVAPANPDPVQVV (SEQ ID NO: 35)
p13-RR Vlep (RRV)-pNB
MGSGPSNFVNKVDGASAPIKEHAIPSLTSDLKDSGIWEVIAGLVALLTFLAFG
FWLFGSLKWKTSQVKHTYRRELIALTRSHVHSTPSGISYV (SEQ ID NO: 36)
p13-RRVtes-ARVch (ARV)
MGSGPSNFVNKVDGASAPIKEHAIPSLTSDLKDSGLLVGLLVLILFFVAAGAA
CWYWGSKAKVKADAARSVFHRELVALSSGKHNAMAPPYDV (SEQ ID NO: 37)
p13-RRVtes-ARVtu
MGSGPSNFVNKVDGASAPIKEHAIPSLTSDLKDSGLLVGLLVLILFFVAAGAA
CWYWGSKAKVKADAARNVFFRELVALNEVKCNAVPPSYRV (SEQ ID NO: 38)
p15-MdRV-MdRV
MGQRHSIVQPPAPPPNAFVESGILGGGFGLIVLLVLVVLIVYCCGSKHSKILSA
VKATDSAVTTLLRDVAPANPDPVQVV (SEQ ID NO: 39)
p15-p15-PsRV
MGQRHSIVQPPAPPPNAFVESGIVSSSTGIIIAVGIFAFIFSFLYKLLGSRAKIKA
DATKNVFRRELIALTTKSGHDRPPSYEV (SEQ ID NO: 40)
p15-pNB-PsRV
MGQRHSIVQPPAPPPNAFVESGYWPHFAIGCGIIVVILLLGLFYCCYGSRAKIK
ADATKNVFRRELIALTTKSGHDRPPSYEV (SEQ ID NO: 41)
p15-RRVtes-ARVtu
MGQRHSIVQPPAPPPNAFVESGLLVGLLVLILFFVAAGAACWYWGSKAKVK
ADAARNVFFRELVALNEVKCNAVPPSYRV (SEQ ID NO: 42)
p15-RRVtes-ARVch (ARV)
MGQRHSIVQPPAPPPNAFVESGLLVGLLVLILFFVAAGAACWYWGSKAKVK
ADAARSVFHRELVALSSGKHNAMAPPYDV (SEQ ID NO: 43)
p16-ARVch (ARV)-ARVch (ARV)
MPCQDTVSLSIQHTSVYVQHSCCVSTTTSASTSATSGYWPYLAAGGGFLLIVII
FALLYCCGSKAKVKADAARSVFHRELVALSSGKHNAMAPPYDV (SEQ ID NO: 44)
P16-RRVlep (RRV)-p10
MPCQDTVSLSIQHTSVYVQHSCCVSTTTSASTSATSGIWEVIAGLVALLTFLA
FGFWLFGSKAKVKADAARSVFHRELVALSSGKHNAMAPPYDV (SEQ ID NO: 45)
p22-MaRV-ARVtu
MGNTISNTVQYTVLQIDRSCCIKTSLTATSEATSSGVYNIIYFFVIGGLILSAGY
GLYGSKAKVKADAARNVFFRELVALNEVKCNAVPPSYRV (SEQ ID NO: 46)
p22-p15-MdRV
MGNTISNTVQYTVLQIDRSCCIKTSLTATSEATSSGIVSSSTGIIIAVGIFAFIFSF
LYKLLGSKHSKILSAVKATDSAVTTLLRDVAPANPDPVQVV (SEQ ID NO: 47)
pNB-ARVch (ARV)-PsRV
MSSDCAKIVSVFGSVHCQSSKNSAGGDLQATSVFTTSGYWPYLAAGGGFLLI
VIIFALLYCCGSRAKIKADATKNVFRRELIALTTKSGHDRPPSYEV (SEQ ID NO: 48)
pNB-p10-p10
MSSDCAKIVSVFGSVHCQSSKNSAGGDLQATSVFTTSGYWPYLAAGGGFLLI
VIIFALLYCCGSKAKVKADAARSVFHRELVALSSGKHNAMAPPYDV (SEQ ID NO: 49)
pNB-p13-ARVch (ARV)
MSSDCAKIVSVFGSVHCQSSKNSAGGDLQATSVFTTSGYLYTIVTAVILLVIL
WFLYRYYGSKAKVKADAARSVFHRELVALSSGKHNAMAPPYDV (SEQ ID NO: 50)
pNB-p22-PsRV
MSSDCAKIVSVFGSVHCQSSKNSAGGDLQATSVFTTSGWAIPPLAICCCCCIC
CTGGLYLVGSRAKIKADATKNVFRRELIALTTKSGHDRPPSYEV (SEQ ID NO: 51)
PVC (VC)-PsRV-MdRV
MGSGPSNFVNHAPGEAIVTGLEKGADKVAGTISHTSGYWPYLAAGGSLLLIV
VLVALFFCCGSKHSKILSAVKATDSAVTTLLRDVAPANPDPVQVV (SEQ ID NO: 52)
pVC (VC)-RRVtes-pNB
MGSGPSNFVNHAPGEAIVTGLEKGADKVAGTISHTSGLLVGLLVLILFFVAAG
AACWYWGSLKWKTSQVKHTYRRELIALTRSHVHSTPSGISYV (SEQ ID NO: 53)
Adhesion Proteins
VCAM-1
GSRKANMKGSYSLVEAQKSKV (SEQ ID NO: 54)
P-Selectin
GSRLSRKGHMYPVRNYSPTEMVCISSLLPDGGEGPSATANGGLSKAKSPGLTPEPREDR
EGDDLTLHSFLP (SEQ ID NO: 55)
L-Selectin
GSRRLKKGKKSKRSMNDPY (SEQ ID NO: 56)
CRTAM
GSKLRKAHVIWKKENEVSEHTLESYRSRSNNEETSSEEKNGQSSHPMRCMNY
ITKLYSEAKTKRKENVQHSKLEEKHIQVPESIV (SEQ ID NO: 57)
ITGB2
GSIAAIVGGTVAGIVLIGILLLVIWKALIHLSDLREYRRFEKEKLKSQWNNDNP
LFKSATTTVMNPKFAES (SEQ ID NO: 58)
ITGB1
GSIIPIVAGVVAGIVLIGLALLLIWKLLMIIHDRREFAKFEKEKMNAKWDTGENPIYKSAV
TTVVNPKYEGK (SEQ ID NO: 59)
E-Selectin
GSRKCLRKAKKFVPASSCQSLESDGSYQKPSYIL (SEQ ID NO: 60)
P-Cadherin
GSKKRKIKEPLLLPEDDTRDNVFYYGEEGGGEEDQDYDITQLHRGLEARPEVVLRNDVA
PTIIPTPMYRPRPANPDEIGNFIIENLKAANTDPTAPPYDTLLVFDYEGSGSDAASLSSLTS
SASDQDQDYDYLNEWGSRFKKLADMYGGGEDD (SEQ ID NO: 61)
CD103
GSKCGFFKRKYQQLNLESIRKAQLKSENLLEEEN (SEQ ID NO: 62)
E-Cadherin
GSILGILGGILALLILILLLLLFLRRRAVVKEPLLPPEDDTRDNVYYYDEEGGGE
EDQDFDLSQLHRGLDARPEVTRNDVAPTLMSVPRYLPRPANPDEIGNFIDENLKAADTD
PTAPPYDSLLVFDYEGSGSEAASLSSLNSSESDKDQDYDYLNEWGNRFKKLADMYGGG
EDD (SEQ ID NO: 63)
N-Cadherin
GSKRRDKERQAKQLLIDPEDDVRDNILKYDEEGGGEEDQDYDLSQLQQPDT
VEPDAIKPVGIRRMDERPIHAEPQYPVRSAAPHPGDIGDFINEGLKAADNDPTAPPYDSL
LVFDYEGSGSTAGSLSSLNSSSSGGEQDYDYLNDWGPRFKKLADMYGGGDD (SEQ ID
NO: 64)
MUC4
GSAFFGIFFGALGGLLLLGVGTFVVLRFWGCSGARFSYFLNSAEALP (SEQ ID
NO: 65)
ICAM1
IVIITVVAAAVIMGTAGLSTYLYNRQRKIKKYRLQQAQKGTPMKPNTQATPP
(SEQ ID NO: 66)
Alanine mutant fusogen sequences
Mut 1
MAAAASNFVNKVDGASAPIKEHAIPSLTSDLKDYLYTIVTAVILLVILWFLYR
YYKDKKARKKKEDILLRLYGRGLNLSRLDPSVICSLGGSAPNLQHRGLERTEDKLVNPFI
(SEQ ID NO: 67)
gccaccatggctgccgcagcgagcaattttgtgaacaaagtagatggggccagcgcccccattaaggagcacgctatcc
catcactgacgagtgatctgaaagactatctctatactatagtcacagcagttatccttctcgtgatcttgtggttcctctacagatattac
aaggacaagaaagcaaggaaaaagaaggaggatatactgctcaggttgtacggtcgcggtctgaacctttctcgccttgatccttcagtaat
ctgcagcttgggaggctctgcacccaacctccaacacaggggacttgaacgaaccgaagataagctcgtaaaccctttcatataa (SEQ ID
NO: 68)
Mut 2
MGSGPAAAANKVDGASAPIKEHAIPSLTSDLKDYLYTIVTAVILLVILWFLYR
YYKDKKARKKKEDILLRLYGRGLNLSRLDPSVICSLGGSAPNLQHRGLERTEDKLVNPFI
(SEQ ID NO: 151)
gccaccatgggttctggacctgctgccgcagcgaacaaagtagatggggccagcgcccccattaaggagcacgctatcc
catcactgacgagtgatctgaaagactatctctatactatagtcacagcagttatccttctcgtgatcttgtggttcctctacagatattac
aaggacaagaaagcaaggaaaaagaaggaggatatactgctcaggttgtacggtcgcggtctgaacctttctcgccttgatccttcagtaat
ctgcagcttgggaggctctgcacccaacctccaacacaggggacttgaacgaaccgaagataagctcgtaaaccctttcatataa (SEQ
ID NO: 69)
Mut 3
MGSGPSNFVAAAAGASAPIKEHAIPSLTSDLKDYLYTIVTAVILLVILWFLYRY
YKDKKARKKKEDILLRLYGRGLNLSRLDPSVICSLGGSAPNLQHRGLERTEDKLVNPFI
(SEQ ID NO: 70)
gccaccatgggttctggacctagcaattttgtggctgccgcagcgggggccagcgcccccattaaggagcacgctatccc
atcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaatc
tgcagcttgggaggctctgcacccaacctccaacacaggggacttgaacgaaccgaagataagctcgtaaaccctttcatataa (SEQ ID
NO: 71)
Mut 4
MGSGPSNFVNKVDAAAAPIKEHAIPSLTSDLKDYLYTIVTAVILLVILWFLYR
YYKDKKARKKKEDILLRLYGRGLNLSRLDPSVICSLGGSAPNLQHRGLERTEDKLVNPFI
(SEQ ID NO: 72)
gccaccatgggttctggacctagcaattttgtgaacaaagtagatgctgccgcagcgcccattaaggagcacgctatccca
tcactgacgagtgatctgaaagactatctctatactatagtcacagcagttatccttctcgtgatcttgtggttcctctacagatattacaa
ggacaagaaagcaaggaaaaagaaggaggatatactgctcaggttgtacggtcgcggtctgaacctttctcgccttgatccttcagtaatct
gcagcttgggaggctctgcacccaacctccaacacaggggacttgaacgaaccgaagataagctcgtaaaccctttcatataa (SEQ ID
NO: 73)
Mut 5
MGSGPSNFVNKVDGASAAAAAHAIPSLTSDLKDYLYTIVTAVILLVILWFLYR
YYKDKKARKKKEDILLRLYGRGLNLSRLDPSVICSLGGSAPNLQHRGLERTEDKLVNPFI
(SEQ ID NO: 74)
gccaccatgggttctggacctagcaattttgtgaacaaagtagatggggccagcgccgctgccgcagcgcacgctatccc
atcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaat
ctgcagcttgggaggctctgcacccaacctccaacacaggggacttgaacgaaccgaagataagctcgtaaaccctttcatataa (SEQ ID
NO: 75)
Mut 6
MGSGPSNFVNKVDGASAPIKEAAAASLTSDLKDYLYTIVTAVILLVILWFLYR
YYKDKKARKKKEDILLRLYGRGLNLSRLDPSVICSLGGSAPNLQHRGLERTEDKLVNPFI
(SEQ ID NO: 76)
gccaccatgggttctggacctagcaattttgtgaacaaagtagatggggccagcgcccccattaaggaggctgccgcagc
gtcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaatc
tgcagcttgggaggctctgcacccaacctccaacacaggggacttgaacgaaccgaagataagctcgtaaaccctttcatataa (SEQ ID
NO: 77)
Mut 7
MGSGPSNFVNKVDGASAPIKEHAIPAAAADLKDYLYTIVTAVILLVILWFLYR
YYKDKKARKKKEDILLRLYGRGLNLSRLDPSVICSLGGSAPNLQHRGLERTEDKLVNPFI
(SEQ ID NO: 78)
gccaccatgggttctggacctagcaattttgtgaacaaagtagatggggccagcgcccccattaaggagcacgctatccca
gctgccgcagcggatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaatc
tgcagcttgggaggctctgcacccaacctccaacacaggggacttgaacgaaccgaagataagctcgtaaaccctttcatataa (SEQ ID
NO: 79)
Mut 8
MGSGPSNFVNKVDGASAPIKEHAIPSLTSAAAAYLYTIVTAVILLVILWFLYR
YYKDKKARKKKEDILLRLYGRGLNLSRLDPSVICSLGGSAPNLQHRGLERTEDKLVNPFI
(SEQ ID NO: 80)
gccaccatgggttctggacctagcaattttgtgaacaaagtagatggggccagcgcccccattaaggagcacgctatccca
tcactgacgagtgctgccgcagcgtatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaat
ctgcagcttgggaggctctgcacccaacctccaacacaggggacttgaacgaaccgaagataagctcgtaaaccctttcatataa (SEQ
ID NO: 81)
Mut 9
MGSGPSNFVNKVDGASAPIKEHAIPSLTSDLKDYLYTIVTAVILLVILWFLYRY
YAAAAARKKKEDILLRLYGRGLNLSRLDPSVICSLGGSAPNLQHRGLERTEDKLVNPFI
(SEQ ID NO: 82)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattacg
ctgccgcagcggcaaggaaaaagaaggaggatatactgctcaggttgtacggtcgcggtctgaacctttctcgccttgatccttcagtaat
ctgcagcttgggaggctctgcacccaacctccaacacaggggacttgaacgaaccgaagataagctcgtaaaccctttcatataa (SEQ
ID NO: 83)
Mut 10
MGSGPSNFVNKVDGASAPIKEHAIPSLTSDLKDYLYTIVTAVILLVILWFLYRY
YKDKKAAAAKEDILLRLYGRGLNLSRLDPSVICSLGGSAPNLQHRGLERTEDKLVNPFI
(SEQ ID NO: 84)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattacaa
ggacaagaaagctgccgcagcgaaggaggatatactgctcaggttgtacggtcgcggtctgaacctttctcgccttgatccttcagtaatct
gcagcttgggaggctctgcacccaacctccaacacaggggacttgaacgaaccgaagataagctcgtaaaccctttcatataa (SEQ ID
NO: 85)
Mut 11
MGSGPSNFVNKVDGASAPIKEHAIPSLTSDLKDYLYTIVTAVILLVILWFLYRY
YKDKKARKKAAAALLRLYGRGLNLSRLDPSVICSLGGSAPNLQHRGLERTEDKLVNPFI
(SEQ ID NO: 86)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaaggctgccgcagcgctgctcaggttgtacggtcgcggtctgaacctttctcgccttgatccttcagtaat
ctgcagcttgggaggctctgcacccaacctccaacacaggggacttgaacgaaccgaagataagctcgtaaaccctttcatataa (SEQ
ID NO: 87)
Mut 12
MGSGPSNFVNKVDGASAPIKEHAIPSLTSDLKDYLYTIVTAVILLVILWFLYRY
YKDKKARKKKEDIAAAAYGRGLNLSRLDPSVICSLGGSAPNLQHRGLERTEDKLVNPFI
(SEQ ID NO: 88)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaagaaggaggatatagctgccgcagcgtacggtcgcggtctgaacctttctcgccttgatccttcagtaatc
tgcagcttgggaggctctgcacccaacctccaacacaggggacttgaacgaaccgaagataagctcgtaaaccctttcatataa (SEQ ID
NO: 89)
Mut 13
MGSGPSNFVNKVDGASAPIKEHAIPSLTSDLKDYLYTIVTAVILLVILWFLYRY
YKDKKARKKKEDILLRLAAAALNLSRLDPSVICSLGGSAPNLQHRGLERTEDKLVNPFI
(SEQ ID NO: 90)
Gccaccatgggttctggacctagcaattttgtgaacaaagtagatggggccagcgcccccattaaggagcacgctatccc
atcactgacgagtgatctgaaagactatctctatactatagtcacagcagttatccttctcgtgatcttgtggttcctctacagatattaca
aggacaagaaagcaaggaaaaagaaggaggatatactgctcaggttggctgccgcagcgctgaacctttctcgccttgatccttcagtaat
ctgcagcttgggaggctctgcacccaacctccaacacaggggacttgaacgaaccgaagataagctcgtaaaccctttcatataa (SEQ ID
NO: 91)
Mut 14
MGSGPSNFVNKVDGASAPIKEHAIPSLTSDLKDYLYTIVTAVILLVILWFLYRY
YKDKKARKKKEDILLRLYGRGAAAARLDPSVICSLGGSAPNLQHRGLERTEDKLVNPFI
(SEQ ID NO: 92)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattacaa
ggacaagaaagcaaggaaaaagaaggaggatatactgctcaggttgtacggtcgcggtgctgccgcagcgCgccttgatccttcagtaatc
tgcagcttgggaggctctgcacccaacctccaacacaggggacttgaacgaaccgaagataagctcgtaaaccctttcatataa (SEQ ID
NO: 93)
Mut 15
MGSGPSNFVNKVDGASAPIKEHAIPSLTSDLKDYLYTIVTAVILLVILWFLYRY
YKDKKARKKKEDILLRLYGRGLNLSAAAASVICSLGGSAPNLQHRGLERTEDKLVNPFI
(SEQ ID NO: 94)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattacaa
ggacaagaaagcaaggaaaaagaaggaggatatactgctcaggttgtacggtcgcggtctgaacctttctgctgccgcagcgtcagtaatct
gcagcttgggaggctctgcacccaacctccaacacaggggacttgaacgaaccgaagataagctcgtaaaccctttcatataa (SEQ ID
NO: 95)
Mut 16
MGSGPSNFVNKVDGASAPIKEHAIPSLTSDLKDYLYTIVTAVILLVILWFLYRY
YKDKKARKKKEDILLRLYGRGLNLSRLDPAAAASLGGSAPNLQHRGLERTEDKLVNPFI
(SEQ ID NO: 96)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatcctgctgccgca
gcgagcttgggaggctctgcacccaacctccaacacaggggacttgaacgaaccgaagataagctcgtaaaccctttcatataa (SEQ ID
NO: 97)
Mut 17
MGSGPSNFVNKVDGASAPIKEHAIPSLTSDLKDYLYTIVTAVILLVILWFLYRY
YKDKKARKKKEDILLRLYGRGLNLSRLDPSVICAAAASAPNLQHRGLERTEDKLVNPFI
(SEQ ID NO: 98)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaat
ctgcgctgccgcagcgtctgcacccaacctccaacacaggggacttgaacgaaccgaagataagctcgtaaaccctttcatataa (SEQ
ID NO: 99)
Mut 18
MGSGPSNFVNKVDGASAPIKEHAIPSLTSDLKDYLYTIVTAVILLVILWFLYRY
YKDKKARKKKEDILLRLYGRGLNLSRLDPSVICSLGGAAAALQHRGLERTEDKLVNPFI
(SEQ ID NO: 100)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaat
ctgcagcttgggaggcgctgccgcagcgctccaacacaggggacttgaacgaaccgaagataagctcgtaaaccctttcatataa (SEQ
ID NO: 101)
Mut 19
MGSGPSNFVNKVDGASAPIKEHAIPSLTSDLKDYLYTIVTAVILLVILWFLYRY
YKDKKARKKKEDILLRLYGRGLNLSRLDPSVICSLGGSAPNAAAAGLERTEDKLVNPFI
(SEQ ID NO: 102)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattac
aaggacaagaaagcaaggaaaaagaaggaggatatactgctcaggttgtacggtcgcggtctgaacctttctcgccttgatccttcagtaa
tctgcagcttgggaggctctgcacccaacgctgccgcagcgggacttgaacgaaccgaagataagctcgtaaaccctttcatataa (SEQ
ID NO: 103)
Mut 20
MGSGPSNFVNKVDGASAPIKEHAIPSLTSDLKDYLYTIVTAVILLVILWFLYRY
YKDKKARKKKEDILLRLYGRGLNLSRLDPSVICSLGGSAPNLQHRAAAATEDKLVNPFI
(SEQ ID NO: 104)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattacaa
ggacaagaaagcaaggaaaaagaaggaggatatactgctcaggttgtacggtcgcggtctgaacctttctcgccttgatccttcagtaatct
gcagcttgggaggctctgcacccaacctccaacacagggctgccgcagcgaccgaagataagctcgtaaaccctttcatataa (SEQ ID
NO: 105)
Mut 21
MGSGPSNFVNKVDGASAPIKEHAIPSLTSDLKDYLYTIVTAVILLVILWFLYRY
YKDKKARKKKEDILLRLYGRGLNLSRLDPSVICSLGGSAPNLQHRGLERTEDKLVNPFI
(SEQ ID NO: 106)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattacaa
ggacaagaaagcaaggaaaaagaaggaggatatactgctcaggttgtacggtcgcggtctgaacctttctcgccttgatccttcagtaatct
tgggaggctctgcacccaacctccaacacaggggacttgaacgagctgccgcagcgctcgtaaaccctttcatataa (SEQ ID
NO: 107)
Mut 22
MGSGPSNFVNKVDGASAPIKEHAIPSLTSDLKDYLYTIVTAVILLVILWFLYRY
YKDKKARKKKEDILLRLYGRGLNLSRLDPSVICSLGGSAPNLQHRGLERTEDKLVNPFI
(SEQ ID NO: 108)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaat
ctgcagcttgggaggctctgcacccaacctccaacacaggggacttgaacgaaccgaagataaggctgccgcagcgttcatataa (SEQ
ID NO: 109)
Mut 23
MGSGPSNFVNKVDGASAPIKEHAIPSLTSDLKDYLYTIVTAVILLVILWFLYRY
YKDKKARKKKEDILLRLYGRGLNLSRLDPSVICSLGGSAPNLQHRGLERTEDKLVNPFI
(SEQ ID NO: 110)
gccaccatgggttctggacctagcaattttgtgaacaaagtagatggggccagcgcccccattaaggagcacgctatccca
tcactgacgagtgatctgaaagactatctctatactatagtcacagcagttatccttctcgtgatcttgtggttcctctacagatattaca
aggacaagaaagcaaggaaaaagaaggaggatatactgctcaggttgtacggtcgcggtctgaacctttctcgccttgatccttcagtaatc
tgcagcttgggaggctctgcacccaacctccaacacaggggacttgaacgaaccgaagataagctcgtaaaccctgctgcctaa (SEQ ID
NO: 111)
P13 Alanine Mutant Sequences
p13_Alanine_5/9
MGSGPSNFVNKVDGASAAAAAHAIPSLTSDLKDYLYTIVTAVILLVILWFLYR
YYAAAAARKKKEDILLRLYGRGLNLSRLDPSVICSLGGSAPNLQHRGLERTEDKLVNPFI
(SEQ ID NO: 112)
p13_Alanine_5/17
MGSGPSNFVNKVDGASAAAAAHAIPSLTSDLKDYLYTIVTAVILLVILWFLYR
YYKDKKARKKKEDILLRLYGRGLNLSRLDPSVICAAAASAPNLQHRGLERTEDKLVNPF
I (SEQ ID NO: 113)
p13_Alanine_5/18
MGSGPSNFVNKVDGASAAAAAHAIPSLTSDLKDYLYTIVTAVILLVILWFLYR
YYKDKKARKKKEDILLRLYGRGLNLSRLDPSVICSLGGAAAALQHRGLERTEDKLVNPF
I (SEQ ID NO: 114)
p13_Alanine_5/20
MGSGPSNFVNKVDGASAAAAAHAIPSLTSDLKDYLYTIVTAVILLVILWFLYR
YYKDKKARKKKEDILLRLYGRGLNLSRLDPSVICSLGGSAPNLQHRAAAATEDKLVNPF
I (SEQ ID NO: 115)
p13_Alanine_9/17
MGSGPSNFVNKVDGASAPIKEHAIPSLTSDLKDYLYTIVTAVILLVILWFLYRY
YAAAAARKKKEDILLRLYGRGLNLSRLDPSVICAAAASAPNLQHRGLERTEDKLVNPFI
(SEQ ID NO: 116)
p13_Alanine_9/18
MGSGPSNFVNKVDGASAPIKEHAIPSLTSDLKDYLYTIVTAVILLVILWFLYRY
YAAAAARKKKEDILLRLYGRGLNLSRLDPSVICSLGGAAAALQHRGLERTEDKLVNPFI
(SEQ ID NO: 117)
p13_Alanine_9/20
MGSGPSNFVNKVDGASAPIKEHAIPSLTSDLKDYLYTIVTAVILLVILWFLYRY
YAAAAARKKKEDILLRLYGRGLNLSRLDPSVICSLGGSAPNLQHRAAAATEDKLVNPFI
(SEQ ID NO: 118)
p13_Alanine_17/18
MGSGPSNFVNKVDGASAPIKEHAIPSLTSDLKDYLYTIVTAVILLVILWFLYRY
YKDKKARKKKEDILLRLYGRGLNLSRLDPSVICAAAAAAAALQHRGLERTEDKLVNPFI
(SEQ ID NO: 119)
p13_Alanine_17/20
MGSGPSNFVNKVDGASAPIKEHAIPSLTSDLKDYLYTIVTAVILLVILWFLYRY
YKDKKARKKKEDILLRLYGRGLNLSRLDPSVICAAAASAPNLQHRAAAATEDKLVNPFI
(SEQ ID NO: 120)
p13_Alanine_18/20
MGSGPSNFVNKVDGASAPIKEHAIPSLTSDLKDYLYTIVTAVILLVILWFLYRY
YKDKKARKKKEDILLRLYGRGLNLSRLDPSVICSLGGAAAALQHRAAAATEDKLVNPFI
(SEQ ID NO: 121)
p13_Alanine_5/9/17
MGSGPSNFVNKVDGASAAAAAHAIPSLTSDLKDYLYTIVTAVILLVILWFLYR
YYAAAAARKKKEDILLRLYGRGLNLSRLDPSVICAAAASAPNLQHRGLERTEDKLVNPF
I (SEQ ID NO: 122)
p13_Alanine_5/9/18
MGSGPSNFVNKVDGASAAAAAHAIPSLTSDLKDYLYTIVTAVILLVILWFLYR
YYAAAAARKKKEDILLRLYGRGLNLSRLDPSVICSLGGAAAALQHRGLERTEDKLVNPF
I (SEQ ID NO: 123)
p13_Alanine_5/9/20
MGSGPSNFVNKVDGASAAAAAHAIPSLTSDLKDYLYTIVTAVILLVILWFLYR
YYAAAAARKKKEDILLRLYGRGLNLSRLDPSVICSLGGSAPNLQHRAAAATEDKLVNPF
I (SEQ ID NO: 124)
p13_Alanine_5/17/18
MGSGPSNFVNKVDGASAAAAAHAIPSLTSDLKDYLYTIVTAVILLVILWFLYR
YYKDKKARKKKEDILLRLYGRGLNLSRLDPSVICAAAAAAAALQHRGLERTEDKLVNP
FI (SEQ ID NO: 125)
p13_Alanine_5/17/20
MGSGPSNFVNKVDGASAAAAAHAIPSLTSDLKDYLYTIVTAVILLVILWFLYR
YYKDKKARKKKEDILLRLYGRGLNLSRLDPSVICAAAASAPNLQHRAAAATEDKLVNP
FI (SEQ ID NO: 126)
p13_Alanine_5/18/20
MGSGPSNFVNKVDGASAAAAAHAIPSLTSDLKDYLYTIVTAVILLVILWFLYR
YYKDKKARKKKEDILLRLYGRGLNLSRLDPSVICSLGGAAAALQHRAAAATEDKLVNP
FI (SEQ ID NO: 127)
p13_Alanine_9/17/18
MGSGPSNFVNKVDGASAPIKEHAIPSLTSDLKDYLYTIVTAVILLVILWFLYRY
YAAAAARKKKEDILLRLYGRGLNLSRLDPSVICAAAAAAAALQHRGLERTEDKLVNPFI
(SEQ ID NO: 128)
p13_Alanine_9/17/20
MGSGPSNFVNKVDGASAPIKEHAIPSLTSDLKDYLYTIVTAVILLVILWFLYRY
YAAAAARKKKEDILLRLYGRGLNLSRLDPSVICAAAASAPNLQHRAAAATEDKLVNPFI
(SEQ ID NO: 129)
p13_Alanine_9/18/20
MGSGPSNFVNKVDGASAPIKEHAIPSLTSDLKDYLYTIVTAVILLVILWFLYRY
YAAAAARKKKEDILLRLYGRGLNLSRLDPSVICSLGGAAAALQHRAAAATEDKLVNPFI
(SEQ ID NO: 130)
p13_Alanine_17/18/20
MGSGPSNFVNKVDGASAPIKEHAIPSLTSDLKDYLYTIVTAVILLVILWFLYRY
YKDKKARKKKEDILLRLYGRGLNLSRLDPSVICAAAAAAAALQHRAAAATEDKLVNPF
I (SEQ ID NO: 131)
p13_Alanine_5/9/17/18
MGSGPSNFVNKVDGASAAAAAHAIPSLTSDLKDYLYTIVTAVILLVILWFLYR
YYAAAAARKKKEDILLRLYGRGLNLSRLDPSVICAAAAAAAALQHRGLERTEDKLVNP
FI (SEQ ID NO: 132)
p13_Alanine_5/9/17/20
MGSGPSNFVNKVDGASAAAAAHAIPSLTSDLKDYLYTIVTAVILLVILWFLYR
YYAAAAARKKKEDILLRLYGRGLNLSRLDPSVICAAAASAPNLQHRAAAATEDKLVNP
FI (SEQ ID NO: 133)
p13_Alanine_5/9/18/20
MGSGPSNFVNKVDGASAAAAAHAIPSLTSDLKDYLYTIVTAVILLVILWFLYR
YYAAAAARKKKEDILLRLYGRGLNLSRLDPSVICSLGGAAAALQHRAAAATEDKLVNP
FI (SEQ ID NO: 134)
p13_Alanine_5/17/18/20
MGSGPSNFVNKVDGASAAAAAHAIPSLTSDLKDYLYTIVTAVILLVILWFLYR
YYKDKKARKKKEDILLRLYGRGLNLSRLDPSVICAAAAAAAALQHRAAAATEDKLVNP
FI (SEQ ID NO: 135)
p13_Alanine_9/17/18/20
MGSGPSNFVNKVDGASAPIKEHAIPSLTSDLKDYLYTIVTAVILLVILWFLYRY
YAAAAARKKKEDILLRLYGRGLNLSRLDPSVICAAAAAAAALQHRAAAATEDKLVNPF
I (SEQ ID NO: 136)
p13_Alanine_5/9/17/18/20
MGSGPSNFVNKVDGASAAAAAHAIPSLTSDLKDYLYTIVTAVILLVILWFLYR
YYAAAAARKKKEDILLRLYGRGLNLSRLDPSVICAAAAAAAALQHRAAAATEDKLVNP
FI (SEQ ID NO: 137)
HA Tag Insertion Site Sequences
P13_HA_1
MGSGPSNFVNKVDYPYDVPDYAGASAPIKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 138)
P13_HA_2
MGSGPSNFVNKVDGYPYDVPDYAASAPIKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 139)
P13_HA_3
MGSGPSNFVNKVDGAYPYDVPDYASAPIKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 140)
P13_HA_4
MGSGPSNFVNKVDGASYPYDVPDYAAPIKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 141)
P13_HA_5
MGSGPSNFVNKVDGASAYPYDVPDYAPIKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 142)
P13_HA_6
MGSGPSNFVNKVDGASAPYPYDVPDYAIKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 143)
P13_HA_7
MGSGPSNFVNKVDGASAPIYPYDVPDYAKEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 144)
P13_HA_8
MGSGPSNFVNKVDGASAPIKYPYDVPDYAEHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 145)
P13_HA_9
MGSGPSNFVNKVDGASAPIKEYPYDVPDYAHAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 146)
P13_HA_10
MGSGPSNFVNKVDGASAPIKEHYPYDVPDYAAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 147)
P13_HA_11
MGSGPSNFVNKVDGASAPIKEHAYPYDVPDYAIPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 148)
P13_HA_12
MGSGPSNFVNKVDGASAPIKEHAIYPYDVPDYAPSLTSDLKDYLYTIVTAVIL
LVILWFLYRYYKDKKARKKKEDILLRLYGRGLNLSRLDPSVICSLGGSAPNLQHRGLER
TEDKLVNPFI (SEQ ID NO: 149)
Miscellaneous
Anti-HA scFv (CD8a leading peptide/anti-HA scFv(1)/CD8a transmembrane domain)
MALPVTALLLPLALLLHAARPAEVKLVESGGGLVKPGGSLKLSCAASGFTFSS
YGMSWVRQTPEKRLEWVATISRGGSYTYYPDSVKGRFTISRDNAKNTLYLQMSSLRSED
TAIYYCARRETYDEKGFAYWGQGTTLTVSSGGGGSGGGGSGGGGSDIVLTQSPASLTVSL
GQRATISCKSSQSLLNSGNQKNYLTWYQQKPGQPPKLLIYWASTRESGIPARFSGSGSGT
DFTLNIHPVEEEDAATYYCQNDNSHPLTFGAGTKLEITTTPAPRPPTPAPTIASQPLSLRPE
ACRPAAGGAVHTRGLDFACDIYIWAPLAGTCGVLLLSLVITLYC (SEQ ID NO: 150)

### REFERENCES

1. Irvine et al., The future of engineered immune cell therapies, Science 378(6622):853-58 (2022).
2. Melenhorst et al., Decade-long leukaemia remissions with persistence of CD4⁺CAR T cells, Nature 602(7897):503-09 (2022).
3. Sadelain et al., Therapeutic T cell engineering, Nature 545(7655):423-31 (2017) (2017).
4. Finck et al., Engineered cellular immunotherapies in cancer and beyond, Nat Med 28(4): 678-89 (2022).
5. Maude et al., Tisagenlecleucel in Children and Young Adults with B-Cell Lymphoblastic Leukemia, New Engl J Medicine 378(5):439-48 (2018).
6. June et al., CAR T cell immunotherapy for human cancer, Science 359(6382): 1361-65 (2018).
7. Allen et al., Synthetic cytokine circuits that drive T cells into immune-excluded tumors, Science 378(6625):eaba1624 (2022).
8. Weber et al., Transient rest restores functionality in exhausted CAR-T cells through epigenetic remodeling, Science 372(6537):eaba1786 (2021).
9. Li et al., Multidimensional control of therapeutic human cell function with synthetic gene circuits, Science 378(6625): 1227-34 (2022).
10. Aguilar et al., Genetic basis of cell-cell fusion mechanisms, Trends Genet 29(7):427-37 (2013).
11. Kanai et al., Cell-cell fusion induced by reovirus FAST proteins enhances replication and pathogenicity of non-enveloped dsRNA viruses, Plos Pathog 15(4):e1007675 (2019).
12. Vance & Lee, Virus and eukaryote fusogen superfamilies, Curr Biol 30(13):R750-R754 (2020).
13. Duncan, Fusogenic Reoviruses and Their Fusion-Associated Small Transmembrane (FAST) Proteins, Annu Rev Virol. 6(1):341-63 (2019).
14. Roybal et al., Precision Tumor Recognition by T Cells With Combinatorial Antigen-Sensing Circuits, Cell 164(4):770-9 (2016).
15. Choe et al., SynNotch-CAR T cells overcome challenges of specificity, heterogeneity, and persistence in treating glioblastoma, Sci Transl Med 13(591):eabe7378 (2021).
16. Zhu et al., Modular design of synthetic receptors for programmed gene regulation in cell therapies, Cell 185(8):1431-43 (2022).
17. Galluzzi et al., Molecular mechanisms of cell death: recommendations of the Nomenclature Committee on Cell Death, Cell Death & Differentiation 25(3):486-541 (2018).
18. Tang et al., The molecular machinery of regulated cell death, Cell Res 29(5):347-64 (2019).
19. Snyder et al., Intratumoral activation of the necroptotic pathway components RIPK1 and RIPK3 potentiates antitumor immunity, Sci Immunol 4(36):eaaw2004 (2019).
20. Wang et al., A bioorthogonal system reveals antitumour immune function of pyroptosis, Nature 579(7799):421-26 (2020).
21. Mantovani et al., IL-1 and IL-I regulatory pathways in cancer progression and therapy, Immunol Rev 281(1):57-61 (2018).
22. Zhao et al., NLRP3 inflammasome activation plays a carcinogenic role through effector cytokine IL-18 in lymphoma, Oncotarget 8(65): 108571-83 (2017).
23. Nickoloff et al., Inflammation and Cancer: Is the Link as Simple as We Think? J Invest Dermatol 124(6):x-xiv (2005).
24. Grivennikov et al., Immunity, Inflammation, and Cancer, Cell 140(6):883-99 (2010).
25. Gaud et al., Regulatory mechanisms in T cell receptor signalling, Nat Rev Immunol 18(8):485-97 (2018).
26. Hughes et al., Transfer of a TCR Gene Derived from a Patient with a Marked Antitumor Response Conveys Highly Active T-Cell Effector Functions, Hum Gene Ther 16(4):457-72 (2005).
27. Morgan et al., High Efficiency TCR Gene Transfer into Primary Human Lymphocytes Affords Avid Recognition of Melanoma Tumor Antigen Glycoprotein 100 and Does Not Alter the Recognition of Autologous Melanoma Antigens, J Immunol 171(6):3287-95 (2003).
28. Sworder et al., Determinants of resistance to engineered T cell therapies targeting CD19 in large B cell lymphomas, Cancer Cell 41(1):210-225.e5 (2023).
29. Ciechonska et al., Efficient Reovirus-and Measles Virus-Mediated Pore Expansion during Syncytium Formation Is Dependent on Annexin A1 and Intracellular Calcium, J Virol 88(11):6137-47 (2014).
30. Yang et al., Polycistronic Genome Segment Evolution and Gain and Loss of FAST Protein Function during Fusogenic Orthoreovirus Speciation, Viruses 12(7):702 (2020).
31. Gao et al., Autophagy, ferroptosis, pyroptosis, and necroptosis in tumor immunotherapy, Signal Transduct Target Ther 7(1):196 (2022).
32. McComb et al., Activation of concurrent apoptosis and necroptosis by SMAC mimetics for the treatment of refractory and relapsed ALL, Sci Transl Med 8(339):339ra70 (2016).
33. Hyrenius-Wittsten et al., SynNotch CAR circuits enhance solid tumor recognition and promote persistent antitumor activity in mouse models, Sci Transl Med 13(591):eabd8836 (2021).
34. Chen et al., CAR-T cell therapy for lung cancer: Potential and perspective, Thorac Cancer 13(7):889-99 (2022).
35. Hu et al., LunX-CAR T Cells as a Targeted Therapy for Non-Small Cell Lung Cancer, Mol Ther-Oncolytics 17:361-70 (2020).
36. Wei et al., PSCA and MUC1 in non-small-cell lung cancer as targets of chimeric antigen receptor T cells, Oncoimmunology 6(3):e1284722 (2017).
37. Zheng et al., Caspase-6 Is a Key Regulator of Innate Immunity, Inflammasome Activation, and Host Defense, Cell 181(3):674-87.e13 (2020).
38. Corrales et al., The host STING pathway at the interface of cancer and immunity, J Clin Investigation 126(7):2404-11 (2016).
39. Chan et al., Evolutionarily related small viral fusogens hijack distinct but modular actin nucleation pathways to drive cell-cell fusion, Proc National Acad Sci 118(1):e2007526118 (2021).
40. Medina & Ravichandran, Do not let death do us part: “find-me” signals in communication between dying cells and the phagocytes, Cell death and differentiation 23(6):979-89 (2016).
41. Medina et al., Metabolites released from apoptotic cells act as tissue messengers, Nature 580(7801):130-35 (2020).
42. Schmitt et al., Colon tumour cell death causes mTOR dependence by paracrine P2X4 stimulation, Nature 612(7939):347-53 (2022).
43. Wang et al., An ER-Targeting Iridium(III) Complex That Induces Immunogenic Cell Death in Non-Small-Cell Lung Cancer, Angew Chem-ger Edit 60(9):4657-4665 (2021).
44. Morris & Chan, Lung Cancer Evolution: What's Immunity Got to Do with It? Cancer Cell 35(5):711-13 (2019).
45. Zappa & Mousa, Non-small cell lung cancer: current treatment and future advances, Transl Lung Cancer Res 5(3):288-300 (2016).
46. Tsherniak et al., Defining a Cancer Dependency Map, Cell 170(3):564-76 (2017).
47. Meyers et al., Computational correction of copy number effect improves specificity of CRISPR-Cas9 essentiality screens in cancer cells, Nat Genet 49(12): 1779-84 (2017).
48. Behan et al., Prioritisation of oncology therapeutic targets using CRISPR-Cas9 screening, Biorxiv 502005 (2018).
49. Colville et al., Death-seq identifies regulators of cell death and senolytic therapies, Biorxiv 486768 (2022).
50. Schapira, Mitochondrial diseases, Lancet 379(9828): 1825-34 (2012).
51. Zhao et al., A genetically encoded probe for imaging nascent and mature HA-tagged proteins in vivo, Nat Commun 10:2947 (2019).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A mammalian cell comprising a nucleotide sequence encoding an exogenous fusogen, wherein the mammalian cell:
a) is an immune effector cell;
b) further comprises a nucleotide sequence encoding a cell-targeting molecule, or both a) and b).

2. The mammalian cell of claim 1, wherein the immune effector cell is selected from the group consisting of a T cell, a monocyte, a B cell, a natural killer (NK) cell, a mononuclear phagocytic cell, and a dendritic cell.

3. The mammalian cell of claim 2, wherein the immune effector cell is:
a) a T cell selected from the group consisting of a helper CD4⁺ T cell, a cytotoxic CD8⁺ T cell, a memory T cell, a regulatory CD4⁺ T cell, an innate-like T cell, a natural killer T cell, a mucosal associated invariant T cell, and a Gamma Delta T cell;
b) a monocyte selected from the group consisting of a CD14⁺⁺CD16 monocyte, a CD14⁺CD16⁺⁺ monocyte, and a CD14⁺⁺CD16⁺ monocyte;
c) a B cell selected from the group consisting of a plasmablast, a plasma cell, a lymphoplasmacytoid cell, a memory B cell, a B-2 cell, a B-1 cell, and a regulatory B cell (Breg);
d) a NK cell selected from the group consisting of a CD56^{BRIGHT} NK cell and a CD56^{DIM} NK cell; or
e) a mononuclear phagocytic cell selected from the group consisting of an adipose tissue macrophage, a monocyte, a Kupffer cell, a sinus histiocyte, an alveolar macrophage (dust cell), a tissue macrophage (histiocyte), a microglial cell, a Hofbauer cell, an intraglomerular mesangial cell, an osteoclast, a Langerhans cell, an epithelioid cell, a red pulp macrophage (sinusoidal lining cell), a peritoneal macrophage, a lysomac, and a perivascular macrophage.

4. The mammalian cell of claim 2, wherein the immune effector cell is a human immune cell or a humanized immune cell.

5. The mammalian cell of claim 1, wherein the exogenous fusogen is a viral fusogen derived from an avian orthoreovirus (ARV), a Nelson Bay virus (NB), a Broome orthoreovirus (BroV), a reptilian orthoreovirus (RRV), a Baboon orthoreovirus (BRV), a group C aquareovirus (AqRV-C), a group A aquareovirus (AqRV-A), a halibut reovirus, a Mahlapitisi orthoreovirus (MaRV), a Muscovy duck orthoreovirus (MdRV), a reptilian Testudine orthoreovirus (RR Vtes), a Piscine orthoreovirus (PsRV), an avian orthoreovirus that infects turkey (ARVtu), a feline rotavirus species I (non-structural protein) NSP-1 (FeRVI), a canine rotavirus species I NSP-1 (CaRVI), a gallinaceous rotavirus species G NSP-1 (GaRVG), an avian rotavirus species G NSP-1 (AvRVG), a caprine rotavirus species B NSP-1 (CaRVB), a porcine rotavirus species B NSP-1 (PoRVB), a human rotavirus species B NSP-1 (HuRVB), a veiled chameleon reovirus (VC), or a variant thereof.

6. The mammalian cell of claim 1, wherein the exogenous fusogen comprises:
a) an extracellular domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof;
b) a transmembrane domain of a viral fusogen derived from an ARV, a Nelson Bay virus, a BroV, a RRV, a BRV, an AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof;
c) an intracellular domain of a viral fusogen derived from an ARV, a Nelson Bay virus, a BroV, a RRV, a BRV, a AqRV-C, an AqRV-A, a halibut reovirus, a MaRV, a MdRV, a RRVtes, a PsRV, an ARVtu, a FeRVI, a CaRVI, a GaRVG, an AvRVG, a CaRVB, a PoRVB, a HuRVB, a VC, or a variant thereof,
or any combination of the foregoing.

7. The mammalian cell of claim 1, wherein the exogenous fusogen comprises an amino acid sequence having at least 90% sequence identity to at least one sequence set forth in SEQ ID NOs: 1-53, 67, 151, 70, 72, 74, 76, 78, 80, 82, 84, 86, 88, 90, 92, 94, 96, 98, 100, 102, 104, 106, 108, 110, and 112-149.

8. The mammalian cell of claim 1, wherein a level of the exogenous fusogen is regulated by:
a) a small molecule selected from the group consisting of tetracycline (TET), doxycycline (DOX), caffeine, 4-hydroxytamoxifen, estrogen, ecdysone, abscisic acid, mifepristone, xylose, FKBP12-rapamycin, and an HCV NS3/4A protease inhibitor;
b) a light-switchable system comprising Phytochrome B (PhyB) and phytochrome interaction factor 6 (PIF6);
c) a light-switchable system comprising GIGANTEA and a light oxygen voltage (LOV) domain of Flavin-Binding Kelch Repeat F-Box 1 (FKF1);
d) a ligand-switchable system selected from the group consisting of a synthetic Notch receptor (synNotch), a modular extracellular sensor, and a synthetic intramembrane proteolysis receptor (SNIPR), or
any combination of the foregoing.

9. The mammalian cell of claim 1, wherein the mammalian cell comprises a nucleotide sequence encoding a cell-targeting molecule that recognizes a cell-surface marker for cancer cells, neurons, oligodendrocytes, microglial cells, neural stem cells, hematopoietic stem cells, astrocytes, or pancreatic beta cells.

10. The mammalian cell of claim 9, wherein the cell-surface marker comprises:
a) B-cell activating factor (BAFF) receptor, B-cell maturation antigen (BCMA), CD3, CD5, CD19, CD20, CD22, CD28, CD30, CD38, CD79B, or programmed cell death protein 1 (PD-1);
b) AXL, B7 homolog 3 protein (B7-H3), carcinoembryonic antigen (CEA), CD70, claudin18.2 (CLDN18.2), delta-like ligand 3 (DLL3), disialoganglioside (GD2), epidermal growth factor receptor (EGFR), glypican-3 (GPC3), guanylyl cyclase C (GUCY2C), human epidermal growth factor receptor 2 (HER2), Kita-Kyushu lung cancer antigen-1 (KK-LC-1), Lewis Y (LEY), mesothelin (MSLN), MUCIN 1 (MUC1), NEW YORK esophageal squamous cell carcinoma 1 (NY-ESO-1), positive programmed death-ligand 1 (PD-L1), prostate specific membrane antigen (PSMA), prostate stem cell antigen (PSCA), receptor-tyrosine-kinase like orphan receptor 1 (ROR1), transforming growth factor beta (TGF-β), Kirsten rat sarcoma virus (KRAS) G12D, melanoma antigen recognized by T cells 1 (MART-1), melanoma-associated antigen 3 (MAGE-A3), tumor protein p53 (TP53), FMS-like tyrosine kinase 3 (FLT3), or alkaline phosphatase placental-like 2 (ALPPL2);
c) L1 cell adhesion molecule (LICAM), neurexin 3, vesicular glutamate transporter 1 (VGLUT1), vesicular inhibitory amino acid transporter (VIAAT), neuroligin 1, neuroligin 2, neural cell adhesion molecule 1 (NCAM1), vesicular acetylcholine transporter (VAChT), folate receptor-1 (FOLR1), gamma-aminobutyric acid B receptor 1 (GABA(b)R1), GABA(b)R2, glutamate ionotropic receptor NMDA type subunit 1 (GRIN1), GRIN2B, or solute carrier family 6 member 4 (SLC6A4);
d) neural/glial antigen 2 (NG2), oligodendrocyte marker 01, oligodendrocyte marker 04, A2B5, or myelin-oligodendrocyte glycoprotein (MOG);
e) P2Y12, macrophage colony-stimulating factor receptor (M-CSFR), or CX3C motif chemokine receptor 1 (CX3CR1);
f) CD133 or CD49F;
g) stem cell antigen-1 (Sca-1), CD27, CD34, CD38, CD43, CD117, or CD150, A2B5, connexin 43, or aquaporin-4 (AQP-4); or
h) CD71 or CD24.

11. The mammalian cell of claim 9, wherein the cell-targeting molecule comprises:
a) a native protein;
b) an antibody or an antigen-binding fragment thereof, a T cell receptor (TCR), an integrin, or a cell adhesion molecule;
c) an integrin selected from the group consisting of integrin beta-1 (ITGβ1), integrin alpha-5 (ITGα5), lymphocyte function-associated antigen 1 (LFA-1), and integrin subunit alpha V (ITGαV);
d) a cell adhesion molecule selected from the group consisting of intercellular adhesion molecule-1 (ICAM-1), vascular cell adhesion protein-1 (VCAM-1), neural cell adhesion molecule 1 (N-CAM1), and platelet endothelial cell adhesion molecule-1 (PE-CAM1);
e) a patient-derived TCR;
f) an engineered protein comprising an antibody or an antigen-binding fragment thereof;
g) an engineered receptor selected from the group consisting of a chimeric antigen receptor (CAR), an engineered T cell receptor (TCR), a synthetic Notch receptor (synNotch), a modular extracellular sensor, a synthetic intramembrane proteolysis receptor (SNIPR), and a cell adhesion molecule;
h) an anti-CD19 or anti-CD27 single chain variable fragment (scFv),
or any combination of the foregoing.

12. The mammalian cell of claim 11, wherein the cell-targeting molecule further comprises an intracellular domain of vascular cell adhesion molecule-1 (VCAM-1), P-Selectin, L-Selectin, MHC class I-restricted T cell-associated molecule (CRTAM), integrin beta2 (ITGβ2), integrin beta1 (ITGβ1), E-Selectin, P-Cadherin, CD103, E-Cadherin, N-Cadherin, mucin-4 (MUC-4), or intercellular adhesion molecule 1 (ICAM1).

13. The mammalian cell of claim 1, further comprising a nucleotide sequence encoding a payload.

14. The mammalian cell of claim 13, wherein:
a) the payload comprises:
i) a therapeutic agent;
ii) a cytokine;
iii) a truncated p15 BID (tBID), caspase 1, receptor-interacting protein kinase-3 (RIPK3), caspase-3, caspase-7, caspase-8, caspase-9, mixed lineage kinase domain-like pseudokinase (MLKL), Gasdermin-D (GSDMD), Gasdermin-E (GSDME), or nerve injury-induced protein 1 (Ninj1);
iv) stimulator of interferon genes (STING), interferon γ, high mobility group box 1 (HMGB1), interleukin-2 (IL-2), IL-12, IL-18, or tumor necrosis factor alpha (TNFα);
v) a checkpoint inhibitor;
vi) an antibody that binds to PD-1, PD-L1, CTLA-4, or LAG3,
or any combination of the foregoing;
b) the payload induces cell death, apoptosis, pyroptosis, necroptosis, an inflammatory immune response, or any combination of the foregoing,
c) the payload is humanized;
d) expression and/or activity of the payload is inducible by a small molecule, a light-switchable system, a ligand-switchable system, or a combination thereof,
or any combination of the foregoing.

15. A method of fusing a mammalian cell with a target cell, comprising co-culturing the mammalian cell of claim 1 with the target cell.

16. The method of claim 15, wherein the target cell is:
a) a neuron, an oligodendrocyte, a microglial cell, a neural stem cell, a hematopoietic stem cell, or a pancreatic beta cell;
b) an immortal cell;
c) a cancer cell;
d) an antibody-producing B lymphocyte;
or any combination of the foregoing.

17. The method of claim 16, wherein the cancer cell is a B-cell lymphoma cell or a lung cancer cell.

18. A method of killing a cancer cell, comprising contacting the cancer cell with an effective dosage of the mammalian cell of claim 1.

19. A composition comprising the mammalian cell of claim 1.

20. A pharmaceutical composition comprising the composition of claim 19 and a pharmaceutically acceptable carrier.

21. A method of treating cancer in a subject in need thereof, comprising administering to the subject a therapeutically effective amount of the pharmaceutical composition of claim 20.

22. A method of screening a library of peptide major histocompatibility complex (pMHC) antigens expressed on the surfaces of a population of mammalian cells, comprising:
a) co-culturing the population of mammalian cells with a population of fusogen-expressing T cells;
b) identifying a fused cell produced by fusion of a cell from the population of mammalian cells and a cell from the population of T cells; and
c) identifying the pMHC antigen expressed on the surface of the fused cell.

23. The method of claim 22, wherein:
a) the pMHC antigen is a tumor antigen, a pathogenic antigen, a bacterial antigen, or a viral antigen;
b) the population of T cells is a population of patient T cells, and the population of mammalian cells is a population of tumor cells from the patient,
or both a) and b).

24. The method of claim 22, further comprising identifying a T cell receptor (TCR) expressed on the surface of the fused cell.

25. A method of screening a library of fusogens useful for mediating cell fusion, comprising:
a) expressing the library of fusogens in a population of mammalian cells, wherein each mammalian cell expresses a unique fusogen;
b) co-culturing the population of mammalian cells with a population of target cells;
c) identifying a fused cell produced by fusion of a cell of the population of mammalian cells and a cell of the population of target cells; and
d) identifying the exogenous fusogen expressed in the fused cell.

26. A polypeptide comprising:
a) an extracellular domain of a viral fusogen derived from a Baboon orthoreovirus (BRV), a Broome orthoreovirus (BroV), an avian orthoreovirus (ARV), a group A aquareovirus (AqRV-A), or a reptilian orthoreovirus (RRV),
b) a transmembrane domain of a viral fusogen derived from a Piscine orthoreovirus (PsRV), a Muscovy duck orthoreovirus (MdRV), a RRV, or an ARV,
c) an intracellular domain of a viral fusogen derived from a RRV, a group C aquareovirus (AqRV-C), a MdRV, a Nelson Bay virus (NB), a Mahlapitisi orthoreovirus (MaRV), or an ARV (avian orthoreovirus),
or any combination of the foregoing.

27. The polypeptide of claim 26, comprising an amino acid sequence having at least 85% sequence identity to at least one sequence set forth in SEQ ID NOs:21-53.

28. The polypeptide of claim 26, comprising an amino acid sequence having 100% sequence identity to a sequence set forth in SEQ ID NOs:21-53.

29. A polynucleotide encoding the polypeptide of claim 26.

30. A mammalian cell comprising the polypeptide of claim 26, a polynucleotide encoding said polypeptide, or both.
